(19) United States
(12) Patent Application Publication
(10) Pub. No.: US 2026/0231678 A1
(43) Pub. Date: Aug. 6, 2026

(54) Composition For Forming Emission Layer, Light-Emitting Device And Electronic Device

(71) Applicant: Samsung Display Co., Ltd., Yongin-si (KR)

(72) Inventor: Hyo Jeong Kim, Yongin-si (KR)

(73) Assignee: Samsung Display Co., Ltd., Yongin-si (KR)

(21) Appl. No.: 19/280,686

(22) Filed: Jul. 25, 2025

(30) Foreign Application Priority Data
Oct. 31, 2024 (KR) 10-2024-0151864

(51) Int. Cl. H10K 85/60; C07D 209/86; C09K 11/02; H10K 50/12; H10K 85/40

(52) U.S. Cl. CPC H10K 85/6572; C07D 209/86; C09K 11/02; H10K 85/40; H10K 85/654; H10K 50/12

(57) ABSTRACT

A composition for forming an emission layer includes a compound represented by Chemical Formula 1 and a compound represent by Chemical Formula 2. A light-emitting device includes a first electrode, a second electrode, and an emission layer disposed between the first electrode and the second electrode. The emission layer includes a composition for forming an emission layer.

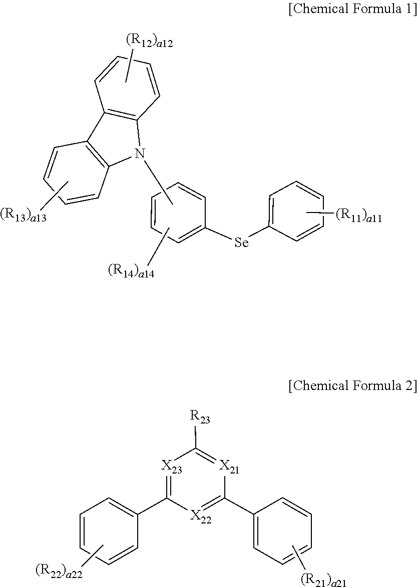

All the variables are described herein.

### PRIORITY

This application claims priority to Korean Patent Application No. 10-2024-0151864 filed on Oct. 31, 2024 in the Korean Intellectual Property Office (KIPO), the entire disclosure of which is incorporated by reference herein.

### TECHNICAL FIELD

Aspects of the present disclosure relate to a composition for forming an emission layer, a light-emitting device and an electronic device.

### BACKGROUND

An organic light-emitting device has a self-luminous property, and may provide improved viewing angle and contrast properties. Additionally, a high response speed and a high luminance may be provided.

The organic light-emitting device may include an emission layer disposed between a first electrode and a second electrode. A hole transferred from the first electrode and an electron transferred from the second electrode may be recombined in the emission layer to generate an exciton. Light emission properties are implemented as the exciton is shifted from an excited state to a ground state.

The emission layer may include a host material and a dopant material for implementing the above-described light emitting mechanism.

### SUMMARY

According to an aspect of the present disclosure, there is provided a composition for forming an emission layer having improved luminous properties.

According to an aspect of the present disclosure, there is provided a light-emitting device having improved luminous and life-span properties.

According to an aspect of the present disclosure, there is provided an electronic device including the light-emitting device.

A composition for forming an emission layer includes a compound represented by Chemical Formula 1 and a compound represent by Chemical Formula 2.

### [Chemical Formula 1]

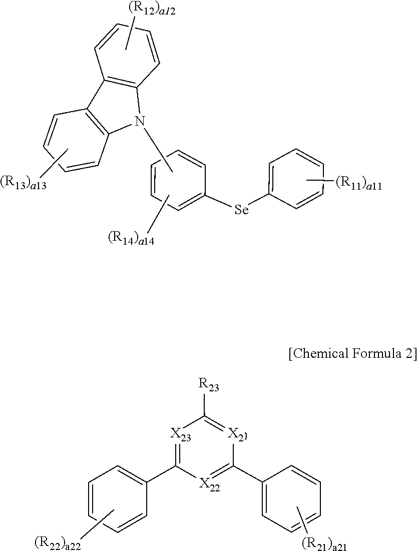

In Chemical Formulae 1 and 2, X₂₁ to X₂₃ are each independently C(R_{1b}) or N. R_{1b} represents hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₈-C₆₀ condensed polycyclic group, —Si(Q₁)(Q₂)(Q₃), —N(Q₁)(Q₂), —B(Q₁)(Q₂), —C(═O)(Q₁), —S(═O)₂(Q₁) or —P(=O)(Q₁)(Q₂). R₁₁ to R₁₄, and R₂₁ to R₂₃ are each independently hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, —Se(Q₁), —Si(Q₁)(Q₂)(Q₃), —N(Q₁)(Q₂), —B(Q₁)(Q₂), —C(═O)(Q₁), —S(═O)₂(Q₁) or —P(═O)(Q₁)(Q₂), or two or more adjacent ones of R₁₁ to R₁₄, and R₂₁ to R₂₃ are combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₃-C₆₀ heterocycloalkenyl ring, a substituted or unsubstituted C₃-C₆₀ aryl ring or a substituted or unsubstituted C₂-C₆₀ heteroaryl ring. Q₁ to Q₃ are each independently hydrogen, deuterium, halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, or a substituted or unsubstituted C₈-C₆₀ a condensed polycyclic group. a11, a21, and a22 are each independently an integer from 0 to 5, and a12 to a14 are each independently an integer from 0 to 4. When a11 to a14, a21, and a22 are each 2 or more, multiple R₁₁ to R₁₄, R₂₁, and R₂₂ are each independently the same or different from each other.

In some aspects, R₁₁ to R₁₄ and R₂₁ to R₂₃ may each independently be hydrogen, deuterium, a substituted or unsubstituted C₁-C₆₀ alkyl group, a substituted or unsubstituted C₂-C₆₀ heteroaryl group, —Se(Q₁) or —Si(Q₁)(Q₂)(Q₃), or two or more adjacent ones of R₁₁ to R₁₄, and R₂₁ to R₂₃ are combined with each other to form a substituted or unsubstituted C₂-C₆₀ heteroaryl ring.

In some aspects, Q₁ to Q₃ may each independently be a substituted or unsubstituted C₆-C₆₀ aryl group or a substituted or unsubstituted C₂-C₆₀ heteroaryl group.

In some aspects, X₂₁ to X₂₃ may be N.

In some aspects, R₂₃ is represented by any one of Chemical Formulae 2A-1 to 2A-6.

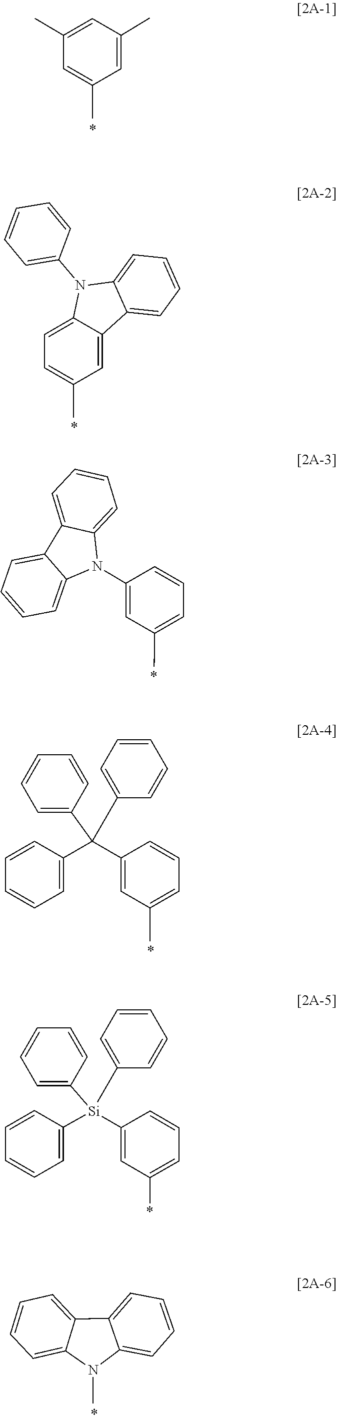

In Chemical Formulae 2A-1 to 2A-6, * indicates a bonding position.

In some aspects, Chemical Formula 1 may be represented by any one of Chemical Formulae 1-1 to 1-8.

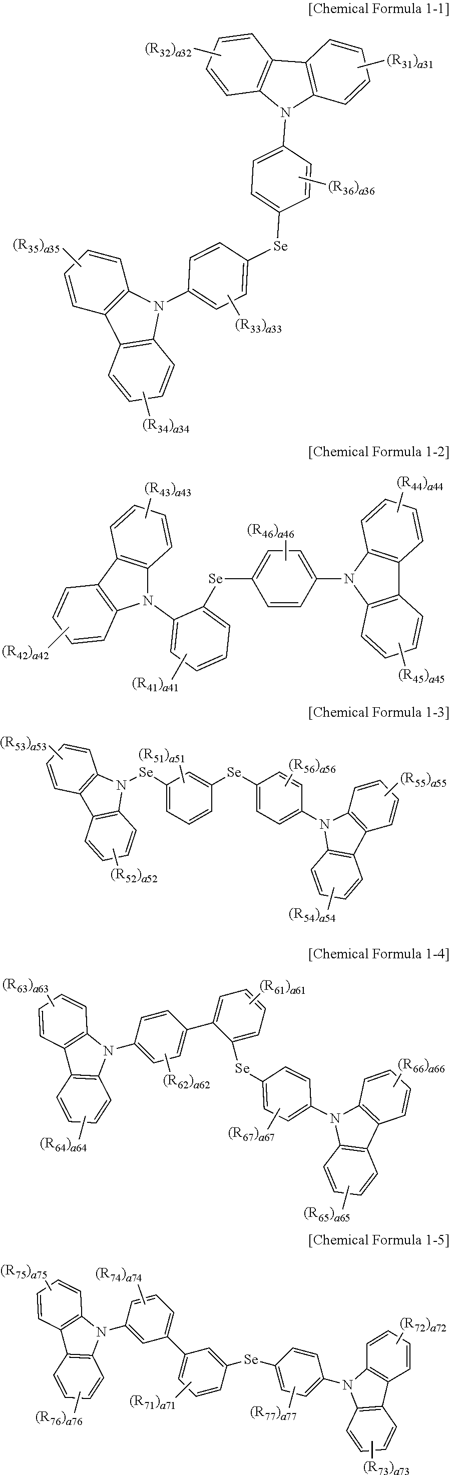

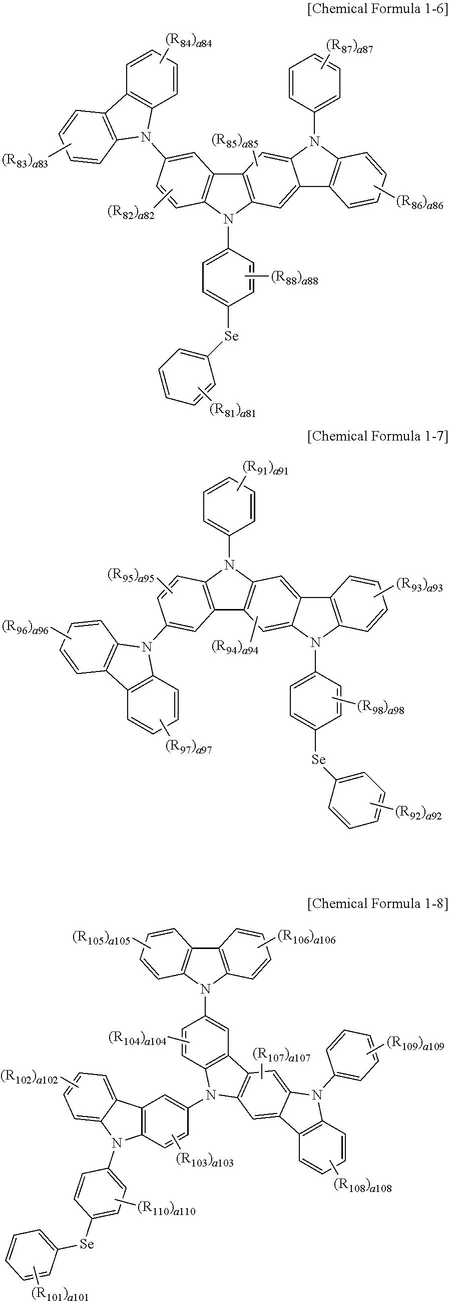

In Chemical Formulae 1-1 to 1-8, R₃₁ to R₃₆, R₄₁ to R₄₆, R₅₁ to R₅₆, R₆₁ to R₆₇, R₇₁ to R₇₇, R₈₁ to R₈₈, R₉₁ to R₉₈, and R₁₀₁ to R₁₁₀ may each independently be hydrogen, deuterium, a substituted or unsubstituted C₁-C₆₀ alkyl group, a substituted or unsubstituted C₂-C₆₀ heteroaryl group, —Se(Q₁) or —Si(Q₁)(Q₂)(Q₃), or two or more of R₃₁ to R₃₆, R₄₁ to R₄₆, R₅₁ to R₅₆, R₆₁ to R₆₇, R₇₁ to R₇₇, R₈₁ to R₈₈, R₉₁ to R₉₈, and R₁₀₁ to R₁₁₀ may be combined with each other to form a substituted or unsubstituted C₂-C₆₀ heteroaryl ring. a31 to a36, a41 to a46, a51 to a56, a61 to a67, a71 to a77, a81, a83, a84, a86, a93, a96 to a98, a102, a105, a106, a108 and a110 may each independently be an integer from 0 to 4. a82, a95, a103, and a104 are each independently be an integer from 0 to 3. a85, a94, and a107 may each independently be an integer from 0 to 2. a87, a91, a92, a101 and a109 are each independently an integer from 0 to 5. When a31 to a36, a41 to a46, a51 to a56, a61 to a67, a71 to a77, a81 to a88, a91 to a98, and a101 to a110 are each 2 or more, multiple R₃₁ to R₃₆, R₄₁ to R₄₆, R₅₁ to R₅₆, R₆₁ to R₆₇, R₇₁ to R₇₇, R₈₁ to R₈₈, R₉₁ to R₉₈, and R₁₀₁ to R₁₁₀ may each independently be the same or different from each other.

In some aspects, R₁₁ to R₁₄ may each independently be hydrogen, deuterium, a substituted or unsubstituted phenyl group, a substituted or unsubstituted carbazole group, a substituted or unsubstituted indolecarbazole group or —Se(Q₁), or two or more adjacent ones among R₁₁ to R₁₄ may be combined with each other to form a substituted or unsubstituted carbazole group.

In some aspects, R₂₁ to R₂₃ may each independently be hydrogen, deuterium, a substituted or unsubstituted methyl group, —Si(Q₁)(Q₂)(Q₃) or a substituted or unsubstituted carbazole group, or two or more adjacent ones among R₂₁ to R₂₃ may be combined with each other to form a substituted or unsubstituted carbazole group.

In some aspects, Chemical Formula 2 may be represented by any one of Chemical Formulae 2-1 to 2-3.

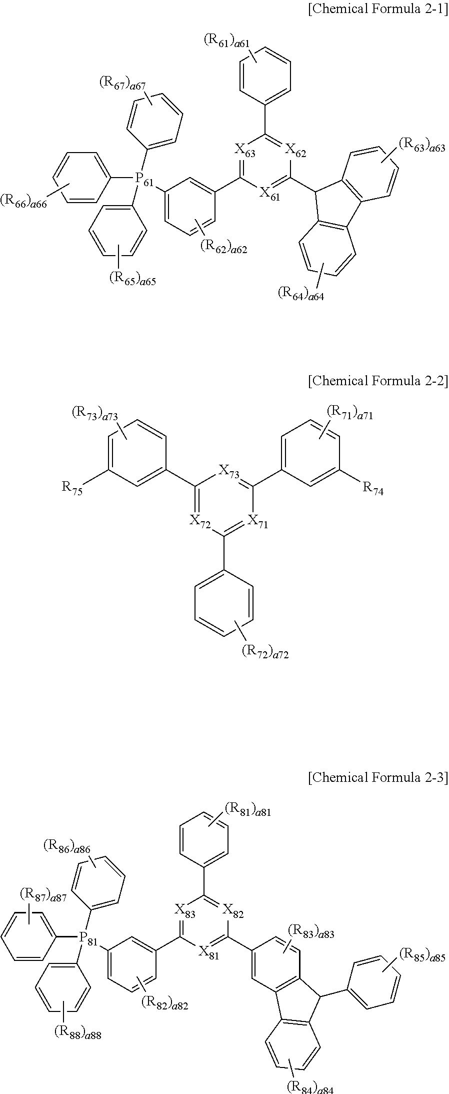

In Chemical Formulae 2-1 to 2-3, X₆₁ to X₆₃, X₇₁ to X₇₃ and X₈₁ to X₈₃ may each independently be C(R_{1b}) or N. R_{1b} may be hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or an unsubstituted C₆-C₆₀ aryloxy group, a substituted or unsubstituted C₈-C₆₀ condensed polycyclic group, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂). P₆₁ and P₈₁ may each independently be C, Si or Ge. R₆₁ to R₆₇, R₇₁ to R₇₅ and R₈₁ to R₈₈ may each independently be hydrogen, deuterium, a substituted or unsubstituted C₁-C₆₀ alkyl group, a substituted or unsubstituted C₂-C₆₀ heteroaryl group or —Si(Q₁)(Q₂)(Q₃), or two or more adjacent ones of R₆₁ to R₆₇, R₇₁ to R₇₅, and R₈₁ to R₈₈ are combined with each other to form a substituted or unsubstituted C₂-C₆₀ heteroaryl ring. Q₁ to Q₃ may each independently be hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, or a substituted or unsubstituted C₈-C₆₀ condensed polycyclic ring. a61, a65 to a67, a72, a81, and a85 to a88 may each independently be an integer from 0 to 5. a62 to a64, a71, a73, a82 and a84 may each independently be an integer from 0 to 4. a83 may be an integer from 0 to 3. When a61 to a67, a71 to a73, and a81 to a88 may be each 2 or more, multiple R₆₁ to R₆₇, R₇₁ to R₇₅, and R₈₁ to R₈₈ may each independently be the same or different from each other.

In some aspects, at least one of R₇₁, R₇₃, R₇₄ and R₇₅ is represented by any one of Chemical Formulae 2-2A-1 to 2-2A-4.

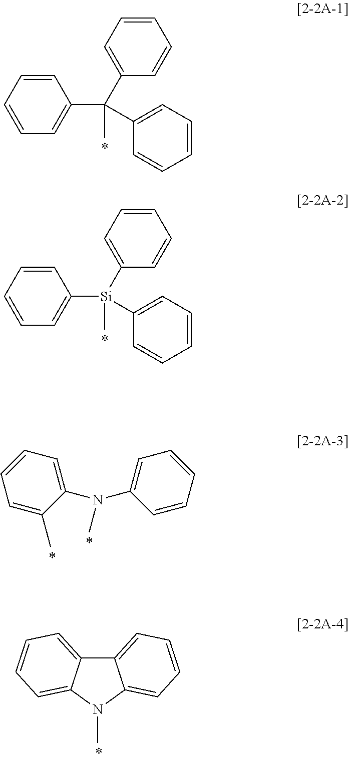

In Chemical Formulae 2-2A-1 to 2-2A-4, * indicates a bonding position.

In some aspects, the compound represented by Chemical Formula 1 may be selected from compounds represented by Chemical Formulae 1-9 to 1-17 below.

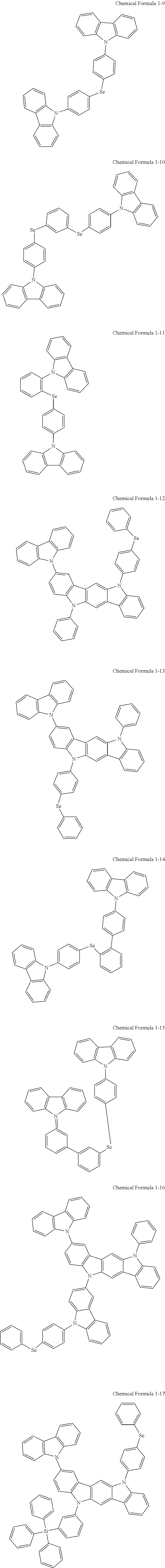

In some aspects, the compound represented by Chemical Formula 2 may be selected from compounds represented by Chemical Formulae 2-4 to 2-15 below.

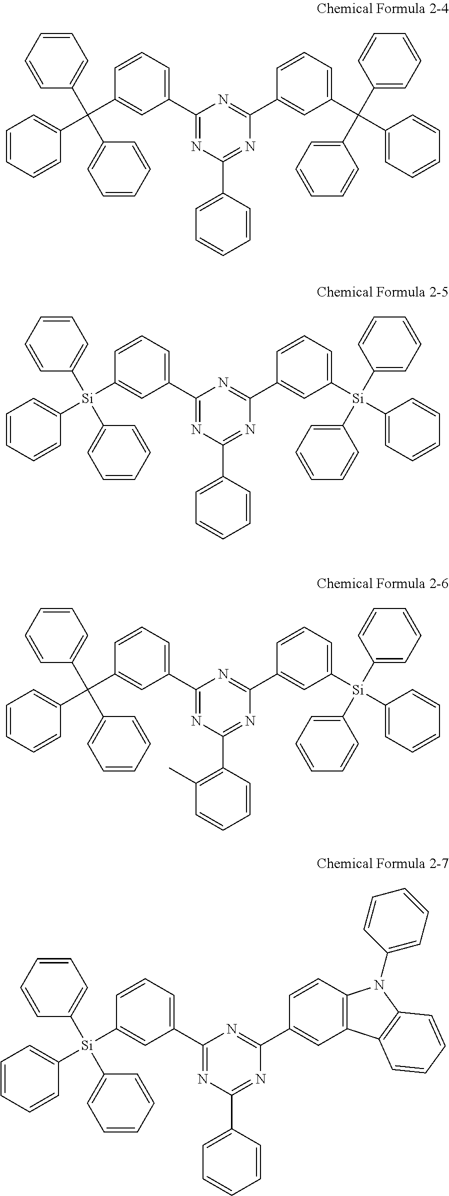 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc([Si](c3ccccc3)(c3ccccc3)c3ccccc3)c2)n1 ; c1ccc(-c2nc(-c3cccc([Si](c4ccccc4)(c4ccccc4)c4ccccc4)c3)nc(-c3ccc4c(c3)c3ccccc3n4-c3ccccc3)n2)cc1]

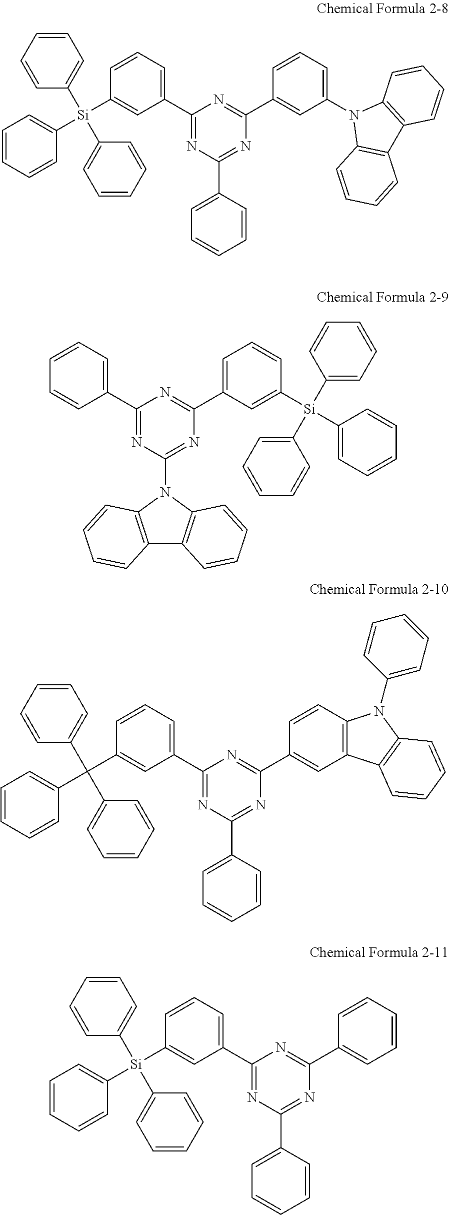 [SMILES: c1ccc(-c2nc(-c3cccc(-n4c5ccccc5c5ccccc54)c3)nc(-c3cccc([Si](c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-n3c4ccccc4c4ccccc43)n2)cc1 ; c1ccc(-c2nc(-c3cccc(C(c4ccccc4)(c4ccccc4)c4ccccc4)c3)nc(-c3ccc4c(c3)c3ccccc3n4-c3ccccc3)n2)cc1 ; c1ccc(-c2nc(-c3ccccc3)nc(-c3cccc([Si](c4ccccc4)(c4ccccc4)c4ccccc4)c3)n2)cc1]

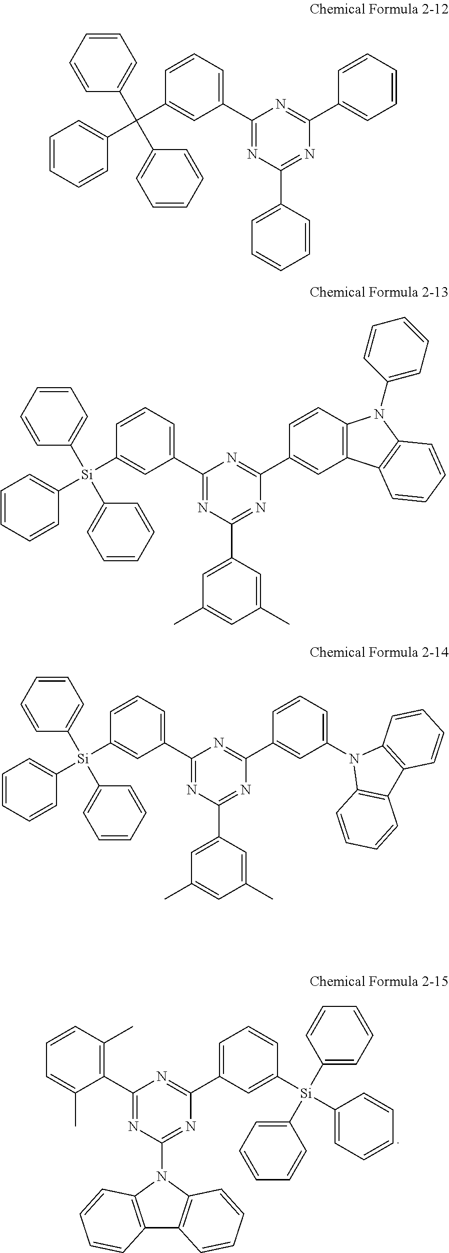 [SMILES: c1ccc(-c2nc(-c3ccccc3)nc(-c3cccc(C(c4ccccc4)(c4ccccc4)c4ccccc4)c3)n2)cc1 ; Cc1cc(C)cc(-c2nc(-c3cccc([Si](c4ccccc4)(c4ccccc4)c4ccccc4)c3)nc(-c3ccc4c(c3)c3ccccc3n4-c3ccccc3)n2)c1 ; Cc1cc(C)cc(-c2nc(-c3cccc(-n4c5ccccc5c5ccccc54)c3)nc(-c3cccc([Si](c4ccccc4)(c4ccccc4)c4ccccc4)c3)n2)c1 ; Cc1cccc(C)c1-c1nc(-c2cccc([Si](c3ccccc3)(c3ccccc3)c3ccccc3)c2)nc(-n2c3ccccc3c3ccccc32)n1 ; "."]

A light-emitting device includes a first electrode, a second electrode, and an emission layer disposed between the first electrode and the second electrode. The emission layer includes a composition for forming an emission layer which includes a compound represented by Chemical Formula 1 and a compound represent by Chemical Formula 2.

A composition for forming an emission layer includes a compound represented by Chemical Formula 1 and a compound represent by Chemical Formula 2.

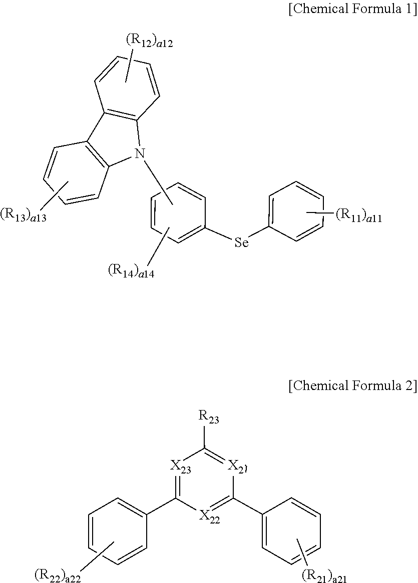

In Chemical Formulae 1 and 2, X₂₁ to X₂₃ are each independently C(R_{1b}) or N. R_{1b} represents hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₈-C₆₀ condensed polycyclic group, —Si(Q₁)(Q₂)(Q₃), —N(Q₁)(Q₂), —B(Q₁)(Q₂), —C(═O)(Q₁), —S(═O)₂(Q₁) or —P(═O)(Q₁)(Q₂). R₁₁ to R₁₄, and R₂₁ to R₂₃ are each independently hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, —Se(Q₁), —Si(Q₁)(Q₂)(Q₃), —N(Q₁)(Q₂), —B(Q₁)(Q₂), —C(═O)(Q₁), —S(═O)₂(Q₁) or —P(═O)(Q₁)(Q₂), or two or more adjacent ones of R₁₁ to R₁₄, and R₂₁ to R₂₃ are combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₃-C₆₀ heterocycloalkenyl ring, a substituted or unsubstituted C₃-C₆₀ aryl ring or a substituted or unsubstituted C₂-C₆₀ heteroaryl ring. Q₁ to Q₃ are each independently hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, or a substituted or unsubstituted C₈-C₆₀ a condensed polycyclic group. a11, a21, and a22 are each independently an integer from 0 to 5, and a12 to a14 are each independently an integer from 0 to 4. When a11 to a14, a21, and a22 are each 2 or more, multiple R₁₁ to R₁₄, R₂₁, and R₂₂ are each independently the same or different from each other.

In some aspects, the composition for forming an emission layer may be included as a host for a blue phosphorescent device or a thermally activated delayed fluorescence (TADF) host.

In some aspects, the emission layer may further include a dopant.

In some aspects, the compound represented by Chemical Formula 1 may be selected from compounds represented by Chemical Formulae 1-9 to 1-17 below.

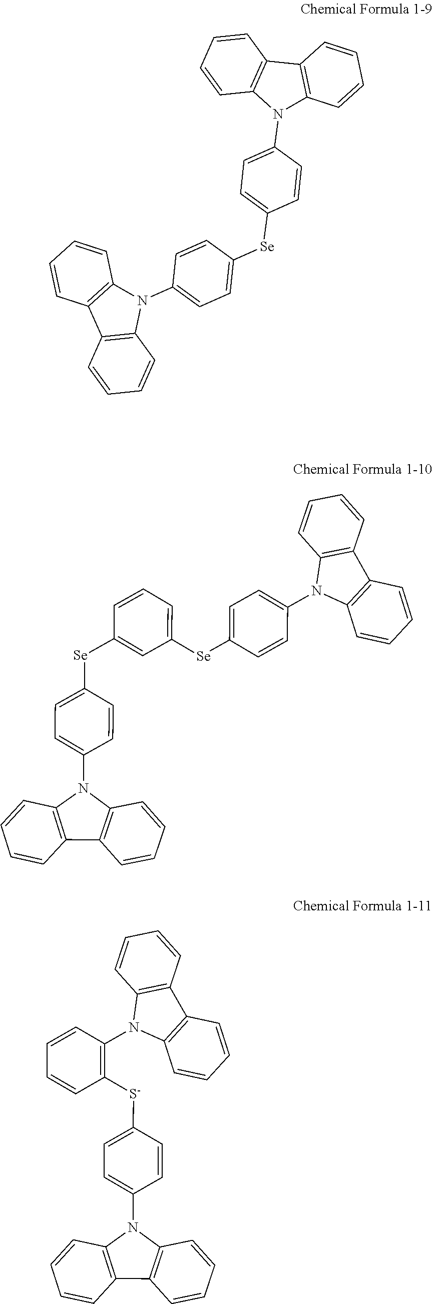

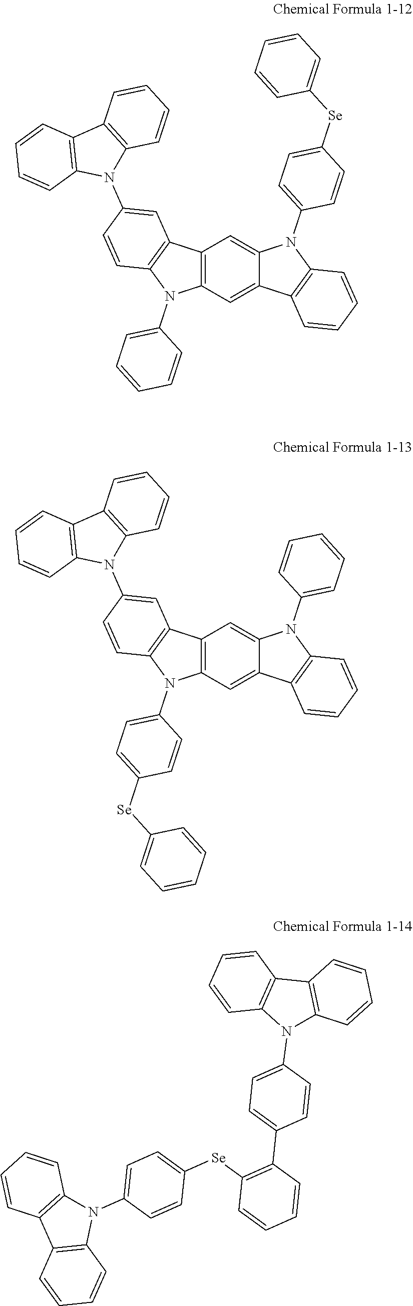 [SMILES: c1ccc([Se]c2ccc(-n3c4ccccc4c4cc5c(cc43)c3cc(-n4c6ccccc6c6ccccc64)ccc3n5-c3ccccc3)cc2)cc1 ; c1ccc([Se]c2ccc(-n3c4ccc(-n5c6ccccc6c6ccccc65)cc4c4cc5c(cc43)c3ccccc3n5-c3ccccc3)cc2)cc1 ; c1ccc(-c2ccc(-n3c4ccccc4c4ccccc43)cc2)c([Se]c2ccc(-n3c4ccccc4c4ccccc43)cc2)c1]

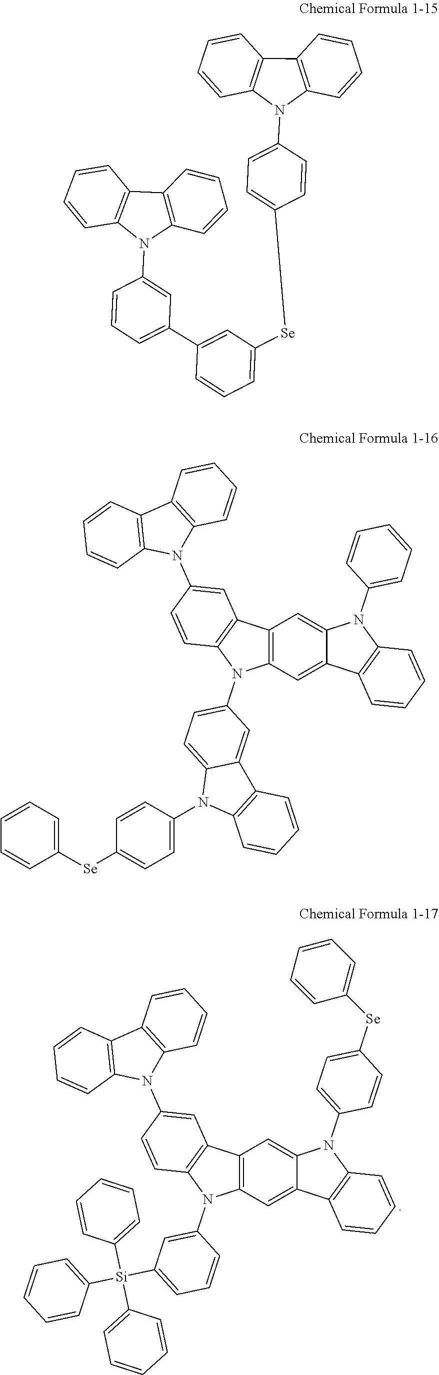 [SMILES: c1cc([Se]c2ccc(-n3c4ccccc4c4ccccc43)cc2)cc(-c2cccc(-n3c4ccccc4c4ccccc43)c2)c1 ; c1ccc([Se]c2ccc(-n3c4ccccc4c4cc(-n5c6ccc(-n7c8ccccc8c8ccccc87)cc6c6cc7c(cc65)c5ccccc5n7-c5ccccc5)ccc43)cc2)cc1 ; c1ccc([Se]c2ccc(-n3c4ccccc4c4cc5c(cc43)c3cc(-n4c6ccccc6c6ccccc64)ccc3n5-c3cccc([Si](c4ccccc4)(c4ccccc4)c4ccccc4)c3)cc2)cc1]

The compound represented by Chemical Formula 2 may be selected from compounds represented by Chemical Formulae 2-4 to 2-15 below.

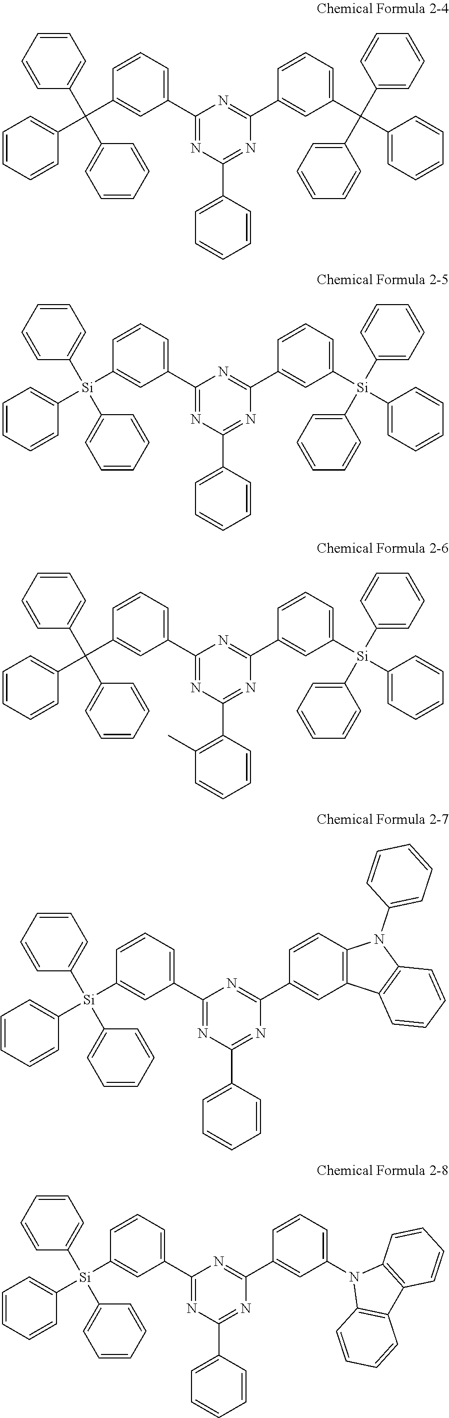

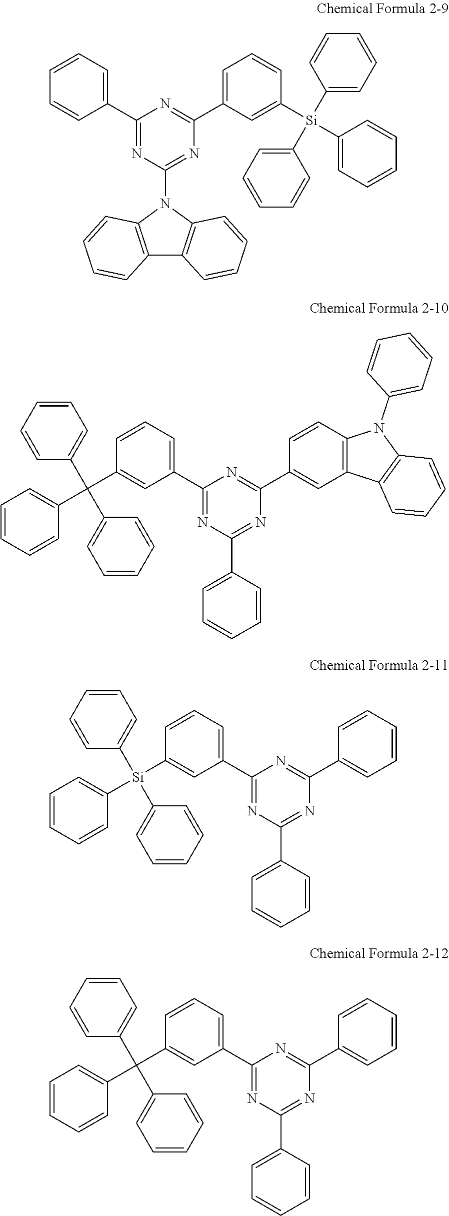 [SMILES: c1ccc(-c2nc(-c3cccc([Si](c4ccccc4)(c4ccccc4)c4ccccc4)c3)nc(-n3c4ccccc4c4ccccc43)n2)cc1 ; c1ccc(-c2nc(-c3cccc(C(c4ccccc4)(c4ccccc4)c4ccccc4)c3)nc(-c3ccc4c(c3)c3ccccc3n4-c3ccccc3)n2)cc1 ; c1ccc(-c2nc(-c3ccccc3)nc(-c3cccc([Si](c4ccccc4)(c4ccccc4)c4ccccc4)c3)n2)cc1 ; c1ccc(-c2nc(-c3ccccc3)nc(-c3cccc(C(c4ccccc4)(c4ccccc4)c4ccccc4)c3)n2)cc1]

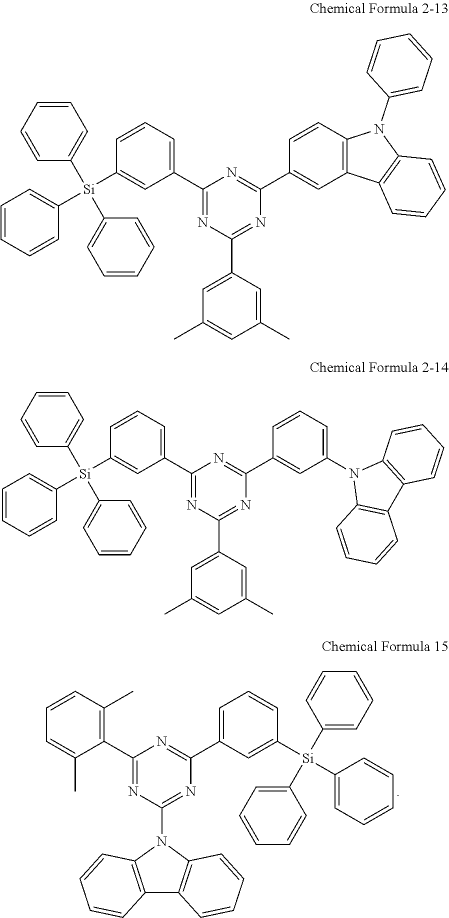 [SMILES: Cc1cc(C)cc(-c2nc(-c3cccc([Si](c4ccccc4)(c4ccccc4)c4ccccc4)c3)nc(-c3ccc4c(c3)c3ccccc3n4-c3ccccc3)n2)c1 ; Cc1cc(C)cc(-c2nc(-c3cccc(-n4c5ccccc5c5ccccc54)c3)nc(-c3cccc([Si](c4ccccc4)(c4ccccc4)c4ccccc4)c3)n2)c1 ; Cc1cccc(C)c1-c1nc(-c2cccc([Si](c3ccccc3)(c3ccccc3)c3ccccc3)c2)nc(-n2c3ccccc3c3ccccc32)n1]

In some aspects, a weight ratio of the compound represented by Chemical Formula 1 and the compound represented by Chemical Formula 2 in the composition for forming the emission layer may be from 1:9 to 9:1.

In some aspects, the emission layer may include a plurality of emission layers, and at least one of the plurality of emission layers may include the composition for forming the emission layer.

An electronic device includes the light-emitting device.

In some aspects, the electronic device may be one of a flat panel display, a curved display, a computer monitor, a medical monitor, a television, a billboard, a light for indoor or outdoor lighting and/or signals, a head-up display, a full or partial transparent display, a flexible display, a rollable display, a foldable display, a stretchable display, a laser printer, a phone, a mobile phone, a tablet, a phablet, a personal information terminal (PDA), a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.

A light-emitting device may include a composition for forming an emission layer according to aspects of the present disclosure to have improved luminous efficiency and life-span properties.

### BRIEF DESCRIPTION OF THE DRAWINGS

FIGS. 1 to 6 are schematic cross-sectional views illustrating light-emitting devices in accordance with aspects of the present disclosure.

FIG. 7 is a schematic cross-sectional view illustrating a display device in accordance with aspects of the present disclosure.

FIG. 8 is a schematic cross-sectional view illustrating a display device in accordance with aspects of the present disclosure.

FIG. 9 is a schematic cross-sectional view illustrating a stack construction of light-emitting structure in a display device in accordance with aspects of the present disclosure.

FIG. 10 is a schematic cross-sectional view illustrating a display device in accordance with aspects of the present disclosure.

FIG. 11 is a schematic cross-sectional view illustrating a display device in accordance with aspects of the present disclosure.

FIG. 12 is a block diagram of an electronic device in accordance with an aspect of the present disclosure.

FIG. 13 is a schematic diagram of an electronic device in accordance with various aspects of the present disclosure.

FIG. 14 is a schematic exploded perspective view illustrating an electronic device in accordance with aspects of the present disclosure.

FIG. 15 is a schematic cross-sectional view illustrating an electronic device in accordance with aspects of the present disclosure.

### DETAILED DESCRIPTION

### Definition of Terminology

In the present specification, the term “substituted or unsubstituted” may refer to being substituted or unsubstituted by one or more substituent selected from the group consisting of, e.g., a deuterium atom, a halogen atom, a cyano group, a nitro group, an amino group, a silyl group, an oxy group, a thio group, a sulfinyl group, a sulfonyl group, a carbonyl group, an ester group, boron, a phosphine oxide group, a phosphine sulfide group, an alkyl group (e.g., a C₁-C₆₀, C₁-C₁₀ alkyl group), an alkenyl group (e.g., a C₂-C₆₀, C₂-C₁₀ alkenyl group), an alkynyl group (e.g., a C₂-C₆₀, C₂-C₁₀ alkynyl group), an alkoxy group (e.g., a C₁-C₆₀, C₁-C₁₀ alkoxy group), a hydrocarbon ring group, an aryl group (e.g., a C₆-C₆₀ aryl group), and a heterocyclic group (e.g., a C₁-C₆₀ heterocyclic group). For example, the term “substituted alkyl group” may refer to a group in which at least one of hydrogen atoms of the alkyl group is substituted with the above-described substituent, and thus the substituent is further bonded to a carbon atom of the alkyl group.

The substituent may include a combination of substituents selected from the groups described above. For example, at least one hydrogen atom in the alkyl group, the aryl group, etc., included as a substituent may be substituted with a deuterium atom, a halogen atom, a cyano group, a nitro group, an amino group, a silyl group, an oxy group, a thio group, a sulfinyl group, a sulfonyl group, a carbonyl group, an ester group, boron, a phosphine oxide group, a phosphine sulfide group, an alkyl group, an alkenyl group, an alkynyl group, a hydrocarbon ring group, an aryl group, a heterocyclic group, or a combination thereof.

In the substituents described above, a multivalent substituent such as an amino group, a phosphine sulfide group, a phosphine oxide group, a sulfinyl group, a sulfonyl group, an oxy group, a carbonyl group, an ester group, etc., may each independently be substituted with a C₁-C₁₀ alkyl group, a C₁-C₁₀ alkenyl group, a C₁-C₁₀ alkynyl group, or a C₆-C₁₀ aryl group.

In the specification, the term “substituted or unsubstituted Cₐ-C_{b} Y group” the range of a to b refers to the number of carbon atoms in an unsubstituted Y group, and may not include the number of carbon atoms of a substituent.

In the specification, an alkyl group may be a monovalent hydrocarbon group in which one hydrogen atom is removed from a linear or branched hydrocarbon group. Examples of an alkyl group may include a methyl group, an ethyl group, a propyl group, a sec-butyl group, a tert-butyl group, an iso-butyl group, a pentyl group, a neopentyl group, a 2-ethyl butyl group, a 3,3-dimethyl butyl group, a hexyl group, a heptyl group, an octyl group, etc.

In the specification, an alkylene group may be a divalent hydrocarbon group in which two hydrogen atoms are removed from a linear or branched hydrocarbon group.

In the specification, an alkenyl group may have the same skeleton as that of an alkyl group, and may be a monovalent hydrocarbon group that includes at least one carbon-carbon double bond. In the specification, an alkenylene group may be a divalent hydrocarbon group in which one hydrogen atom is further removed from an alkenyl group.

In the specification, an alkynyl group may have the same skeleton as that of an alkyl group, and may be a monovalent hydrocarbon group that includes at least one carbon-carbon triple bond. In the specification, an alkynylene group may be a divalent hydrocarbon group in which one hydrogen atom is further removed from an alkynyl group.

In the specification, an aryl group may be a monovalent hydrocarbon group in which one hydrogen atom is removed from a hydrocarbon group having an aromatic structure. The definition of an aryl group may also encompass a group in which multiple aromatic rings are directly connected, such as a biphenyl group. Examples of an aryl group may include, e.g., a phenyl group, a naphthyl group, an anthracenyl group, a phenanthrenyl group, a pyrenyl group, a fluorenyl group, a tetracenyl group, a biphenyl group, a terphenyl group, a quaterphenyl group, a chrysenyl group, etc.

In the specification, a group in which two or more aryl rings are condensed to each other or linked to each other by an alicyclic hydrocarbon ring, such as a fluorenyl group, can be encompassed in the definition of an aryl group.

For example, a biphenyl group may be interpreted as an aryl group, or may be interpreted as a phenyl group that is substituted with a phenyl group.

In the specification, an arylene group may be a divalent hydrocarbon group in which two hydrogen atoms are removed from an aryl group.

In the specification, a heteroaryl group may be a monovalent group having an aromatic structure that includes at least one heteroatom such as B, O, P, S, or Si as a ring-forming atom. In the specification, a heteroarylene group may be a divalent group having an aromatic structure that includes at least one heteroatom such as B, O, P, S, or Si as a ring-forming atom. When a heteroaryl group or a heteroarylene group includes two or more heteroatoms, the two or more heteroatoms may be the same as or different from each other.

In the specification, a group in which two or more aryl rings are condensed or linked to a non-aromatic heterocyclic ring, such as a carbazole group, can also be encompassed in the definition of a heteroaryl group.

In the specification, the term “cyclic group” may encompass a monocyclic group or a polycyclic group, and may also encompass an alicyclic ring or an aromatic ring.

In the specification, the term “polycyclic group” may be a group in which two or more rings are connected to each other or condensed to each other through one or more atoms. For example, a polycyclic structure may include a bicyclic structure through a bridge carbon, a spiro structure, a fused structure, etc.

In the specification, the term “condensed group” or “condensed ring structure” may each be a group in which two or more adjacent rings share two or more atoms among the above-described polycyclic structures. Examples of a condensed ring structure may include naphthalene, anthracene, phenanthrene, fluorene, pyrene, benzopyrene, pentacene, polyacene, helicene, etc.

In the specification, the term “carbocyclic group (e.g., C₃-C₆₀ carbocyclic group)” may be a cyclic group in which carbon atoms are the only ring-forming atoms. In the specification, a heterocyclic group (e.g., a C₁-C₆₀ heterocyclic group) may be a cyclic group that includes at least one heteroatom as a ring-forming atom, in addition to carbon atoms.

In the specification, a carbocyclic group and a heterocyclic group may each independently be a monocyclic group that consists of one ring or a polycyclic group in which two or more rings are condensed with each other.

### Light-Emitting Device

According to aspects of the present disclosure, a light-emitting device may include a first electrode, a second electrode, and an emission layer including a composition for forming an emission layer disposed between the first electrode and the second electrode. The emission layer may include a composition for forming an emission layer,

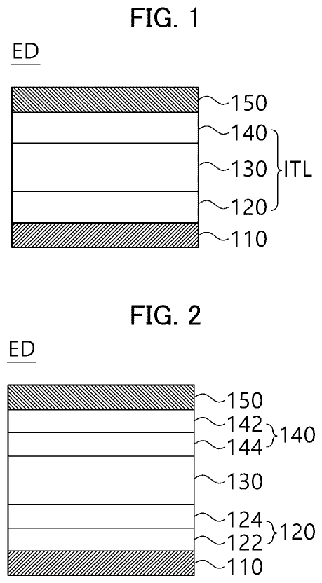

FIGS. 1 to 6 are schematic cross-sectional views illustrating light-emitting devices in accordance with aspects of the present disclosure.

Referring to FIG. 1, a light-emitting device ED may include a first electrode 110, a second electrode 150, and an intermediate layer ITL interposed between the first electrode 110 and the second electrode 150. The intermediate layer ITL may include an emission layer 130. The intermediate layer ITL may further include a hole transfer region 120 and an electron transfer region 140.

### First Electrode/Second Electrode

The first electrode 110 may be an anode or a cathode. In some aspects, the first electrode 110 may be an anode, and may serve as a pixel electrode. In this case, the first electrode 110 may include a conductive material with a high work function that promotes hole injection.

In an aspect, the first electrode 110 may be a transmissive electrode. The first electrode 110 may include a transparent conductive oxide such as indium tin oxide (ITO), indium zinc oxide (IZO), zinc oxide (ZnO), indium tin oxide (ITZO), etc.

In an aspect, the first electrode 110 may be a translucent electrode or a reflective electrode. The first electrode 110 may include at least one of Ag, Mg, Cu, Al, Pt, Pd, Au, Ni, Nd, Ir, Cr, Li, Ca, LiF, Mo, Ti, W, In, Sn, Zn, or an alloy containing at least two therefrom. For example, the first electrode 110 may include Li, Ca, LiF/Ca (a stacked structure of LiF and Ca), LiF/Al (a stacked structure of LiF and Al), a mixture of Ag and Mg, etc.

The first electrode 110 may have a single-layered structure or a multi-layered structure. For example, the first electrode 110 may have a triple-layered structure of ITO/Ag/ITO.

A thickness of the first electrode 110 may be in a range of about 700 Å to about 10,000 Å. For example, the thickness of the first electrode 110 may be in a range of about 1,000 Å to about 3,000 Å.

The second electrode 150 may be a cathode or an anode. In some aspects, the second electrode 150 may serve as an electron injection electrode or as a cathode. The second electrode 150 may include a metal, an alloy, an electrically conductive compound, etc., having a low work function.

For example, the second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, etc. The second electrode 150 may include one of the aforementioned materials, or a combination thereof.

The second electrode 150 may be a transmissive electrode, a translucent electrode, or a reflective electrode. The second electrode 150 may have a single-layered structure or a multi-layered structure.

### Composition for Forming Emission Layer/Emission Layer

In some aspects, a composition for forming an emission layer may include a compound represented by Chemical Formula 1 and a compound represented by Chemical Formula 2.

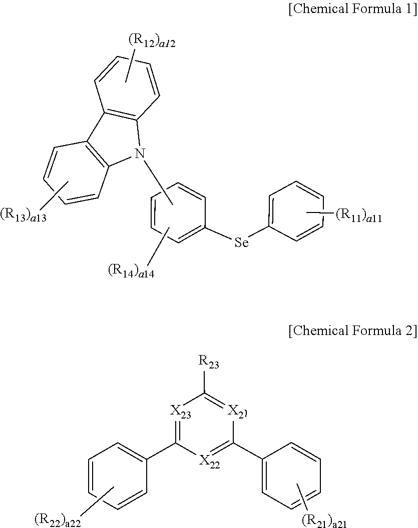

In Chemical Formulae 1 and 2, X₂₁ to X₂₃ may each independently be C(R_{1b}) or N. R_{1b} may represent hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₈-C₆₀ condensed polycyclic group, —Si(Q₁)(Q₂)(Q₃), —N(Q₁)(Q₂), —B(Q₁)(Q₂), —C(═O)(Q₁), —S(═O)₂ (Q₁), or —P(═O)(Q₁)(Q₂)

In an aspect, R₁₁ to R₁₄, and R₂₁ to R₂₃ may each independently be hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, —Se(Q₁), —Si(Q₁)(Q₂)(Q₃), —N(Q₁)(Q₂), —B(Q₁)(Q₂), —C(═O)(Q₁), —S(═O)₂(Q₁) or —P(═O)(Q₁)(Q₂).

In an aspect, two or more adjacent ones of R₁₁ to R₁₄, and R₂₁ to R₂₃ may be combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₃-C₆₀ heterocycloalkenyl ring, a substituted or unsubstituted C₃-C₆₀ aryl ring or a substituted or unsubstituted C₂-C₆₀ heteroaryl ring.

Q₁ to Q₃ may each independently be hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, or a substituted or unsubstituted C₈-C₆₀ condensed polycyclic group.

a11, a21, and a22 may each independently be an integer from 0 to 5, and a12 to a14 may each independently be an integer from 0 to 4.

When a11 to a14, a21, and a22 are each 2 or more, multiple R₁₁ to R₁₄, R₂₁, and R₂₂ may each independently be the same or different from each other.

The composition for forming the emission layer may include the compounds represented by Chemical Formulae 1 and 2, so that a charge balance in the emission layer may be optimized and luminous efficiency and life-span properties may be improved.

The compound represented by Chemical Formula 1 includes Se, not a carbazole group, as a core, and reverse intersystem crossing (RISC) may occur rapidly by a heavy atom effect. Accordingly, luminous efficiency may be improved through a roll-off enhancement, and life-span properties may be improved through a decrease in a triplet concentration.

The compound represented by Chemical Formula 1 may have enhanced hole mobility. The compound represented by Chemical Formula 2 may have enhanced electron mobility, so that unnecessary interaction with a dopant may be suppressed compared to that of a carbazole compound.

Various substituents may be introduced into the compound represented by Chemical Formula 1 and the compound represented by Chemical Formula 2, so that an energy level of the light-emitting device and a polarity of a molecule may be easily controlled.

In an aspect, the composition for forming an emission layer may be applied to the light-emitting device to lower a driving voltage. The composition for forming an emission layer may be applied as a host for a high-efficiency blue phosphorescent device and a thermally activated delayed fluorescence (TADF) host.

In some aspects, a deuterium substitution ratio of at least one of the compound represented by Chemical Formula 1 and the compound represented by Chemical Formula 2 may be 0% or more, 1% or more, 2% or more, 3% or more, or 11% or more, and may be less than 55%. When at least one of the compound represented by Chemical Formula 1 and the compound represented by Chemical Formula 2 is substituted with deuterium, life-span properties may be further improved.

In an aspect, R₁₁ to R₁₄ and R₂₁ to R₂₃ may each independently be hydrogen, deuterium, a substituted or unsubstituted C₁-C₆₀ alkyl group, a substituted or unsubstituted C₂-C₆₀ heteroaryl group, —Se(Q₁) or —Si(Q₁)(Q₂)(Q₃). In an aspect, two or more adjacent ones of R₁₁ to R₁₄, and R₂₁ to R₂₃ may be combined with each other to form a substituted or unsubstituted C₂-C₆₀ heteroaryl ring.

In an aspect, R₁₁ to R₁₄ and R₂₁ to R₂₃ may each independently be hydrogen, deuterium, a substituted or unsubstituted C₁-C₅₀ alkyl group, a substituted or unsubstituted C₂-C₅₀ heteroaryl group, —Se(Q₁) or —Si(Q₁)(Q₂)(Q₃). In an aspect, two or more adjacent ones of R₁₁ to R₁₄, and R₂₁ to R₂₃ may be combined with each other to form a substituted or unsubstituted C₂-C₅₀ heteroaryl ring.

In an aspect, R₁₁ to R₁₄ and R₂₁ to R₂₃ may each independently be hydrogen, deuterium, a substituted or unsubstituted C₁-C₄₀ alkyl group, a substituted or unsubstituted C₂-C₄₀ heteroaryl group, —Se(Q₁) or —Si(Q₁)(Q₂)(Q₃). In an aspect, two or more adjacent ones of R₁₁ to R₁₄, and R₂₁ to R₂₃ may be combined with each other to form a substituted or unsubstituted C₂-C₄₀ heteroaryl ring.

In an aspect, R₁₁ to R₁₄ and R₂₁ to R₂₃ may each independently be hydrogen, deuterium, a substituted or unsubstituted C₁-C₃₀ alkyl group, a substituted or unsubstituted C₂-C₃₀ heteroaryl group, —Se(Q₁) or —Si(Q₁)(Q₂)(Q₃). In an aspect, two or more adjacent ones of R₁₁ to R₁₄, and R₂₁ to R₂₃ may be combined with each other to form a substituted or unsubstituted C₂-C₃₀ heteroaryl ring.

In an aspect, R₁₁ to R₁₄ and R₂₁ to R₂₃ may each independently be hydrogen, deuterium, a substituted or unsubstituted C₁-C₂₀ alkyl group, a substituted or unsubstituted C₂-C₂₀ heteroaryl group, —Se(Q₁) or —Si(Q₁)(Q₂)(Q₃). In an aspect, two or more adjacent ones of R₁₁ to R₁₄, and R₂₁ to R₂₃ may be combined with each other to form a substituted or unsubstituted C₂-C₂₀ heteroaryl ring.

In an aspect, R₁₁ to R₁₄ and R₂₁ to R₂₃ may each independently be hydrogen, deuterium, a substituted or unsubstituted C₁-C₁₀ alkyl group, a substituted or unsubstituted C₂-C₁₀ heteroaryl group, —Se(Q₁) or —Si(Q₁)(Q₂)(Q₃). In an aspect, two or more adjacent ones of R₁₁ to R₁₄, and R₂₁ to R₂₃ may be combined with each other to form a substituted or unsubstituted C₂-C₁₀ heteroaryl ring.

In one aspect, R₁₁ to R₁₄, and R₂₁ to R₂₃ may each independently be hydrogen, deuterium, a substituted or unsubstituted methyl group, a substituted or unsubstituted carbazole group, a substituted or unsubstituted indolecarbazole group, —Si(Q₁)(Q₂)(Q₃) or —Se(Q₁). In an aspect, two or more adjacent ones of R₁₁ to R₁₄, and R₂₁ to R₂₃ may be combined with each other to form a substituted or unsubstituted carbazole group.

In an aspect, R₁₁ to R₁₄ may each independently be hydrogen, deuterium, a substituted or unsubstituted phenyl group, a substituted or unsubstituted carbazole group, a substituted or unsubstituted indolecarbazole group, or —Se(Q₁). In an aspect, two or more adjacent ones of R₁₁ to R₁₄ may be combined with each other to form a substituted or unsubstituted carbazole group.

In an aspect, R₁₁ to R₁₄ may each independently be hydrogen, deuterium, a phenyl group substituted with a carbazole group, a carbazole group, a phenyl group and an indolecarbazole group substituted with a carbazole group, or —Se(Q₁). In an aspect, two or more adjacent ones of R₁₁ to R₁₄ may be combined with each other to form a carbazole group substituted with a phenyl group and a carbazole group, or a carbazole group substituted with a phenyl group substituted with a triphenylsilyl group and a carbazole group, and Q₁ may be a phenyl group substituted with a carbazole group.

In some aspects, R₁₁ may be hydrogen, deuterium, a phenyl group substituted with a carbazole group, a carbazole group, or —Se(Q₁), and Q₁ may be a phenyl group substituted with a carbazole group.

In an aspect, R₁₂ and R₁₃ may each independently be hydrogen, deuterium, a carbazole group, or an indolecarbazole group substituted with a phenyl group and a carbazole group. In an aspect, two or more adjacent ones of R₁₂ and R₁₃ may be combined with each other to form a carbazole group substituted with a phenyl group, a carbazole group substituted with a phenyl group and a carbazole group, or a carbazole group substituted with a phenyl group substituted with a triphenylsilyl group and a carbazole group.

In an aspect, R₁₄ may be hydrogen or deuterium.

In an aspect, R₂₁ to R₂₃ may each independently be hydrogen, deuterium, a substituted or unsubstituted methyl group, —Si(Q₁)(Q₂)(Q₃), or a substituted or unsubstituted carbazole group. In an aspect, two or more adjacent ones of R₂₁ to R₂₃ may be combined with each other to form a substituted or unsubstituted carbazole group.

In an aspect, R₂₁ to R₂₃ may each independently be hydrogen, deuterium, a methyl group, a methyl group substituted with a phenyl group, a phenyl group substituted with a methyl group, a carbazole group, or —Si(Q₁)(Q₂)(Q₃). In an aspect, two or more adjacent ones of R₂₁ to R₂₃ may be bonded to each other to form a carbazole group substituted with a phenyl group, and Q₁ to Q₃ may be a phenyl group.

In an aspect, R₂₁ and R₂₂ may each independently be hydrogen, deuterium, a methyl group, a methyl group substituted with a phenyl group, a carbazole group, or —Si(Q₁)(Q₂)(Q₃). In an aspect, two or more adjacent ones of R₂₁ and R₂₂ may be combined to form a carbazole group substituted with a phenyl group, and Q₁ to Q₃ may be a phenyl group.

In an aspect, R₂₃ may be a phenyl group, a phenyl group substituted with a methyl group, or a carbazole group.

In some aspects, Q₁ to Q₃ may each independently be a substituted or unsubstituted C₆-C₆₀ aryl group or a substituted or unsubstituted C₂-C₆₀ heteroaryl group.

In some aspects, Q₁ to Q₃ may each independently be a substituted or unsubstituted C₆-C₅₀ aryl group or a substituted or unsubstituted C₂-C₅₀ heteroaryl group.

In some aspects, Q₁ to Q₃ may each independently be a substituted or unsubstituted C₆-C₄₀ aryl group or a substituted or unsubstituted C₂-C₄₀ heteroaryl group.

In some aspects, Q₁ to Q₃ may each independently be a substituted or unsubstituted C₆-C₃₀ aryl group or a substituted or unsubstituted C₂-C₃₀ heteroaryl group.

In some aspects, Q₁ to Q₃ may each independently be a substituted or unsubstituted C₆-C₂₀ aryl group or a substituted or unsubstituted C₂-C₂₀ heteroaryl group.

In some aspects, Q₁ to Q₃ may each independently be a substituted or unsubstituted phenyl group or a substituted or unsubstituted carbazole group.

In some aspects, Q₁ to Q₃ may each independently be a phenyl group or a carbazole group.

In some aspects, X₂₁ to X₂₃ may be N.

In some aspects, X₂₁ to X₂₃ may be C(R_{1b}), and the above descriptions of R_{1b} may be equally applied.

In some aspects, R₂₃ may be represented by any one of Chemical Formulae 2A-1 to 2A-6.

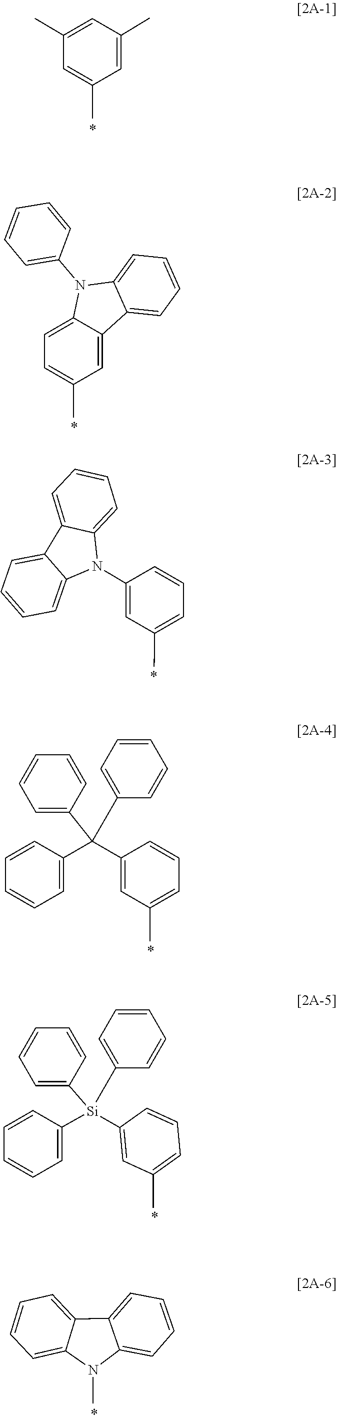

In Chemical Formulae 2A-1 to 2A-6, * indicates a bonding position.

In some aspects, Chemical Formula 1 may be represented by any one of Chemical Formulae 1-1 to 1-8.

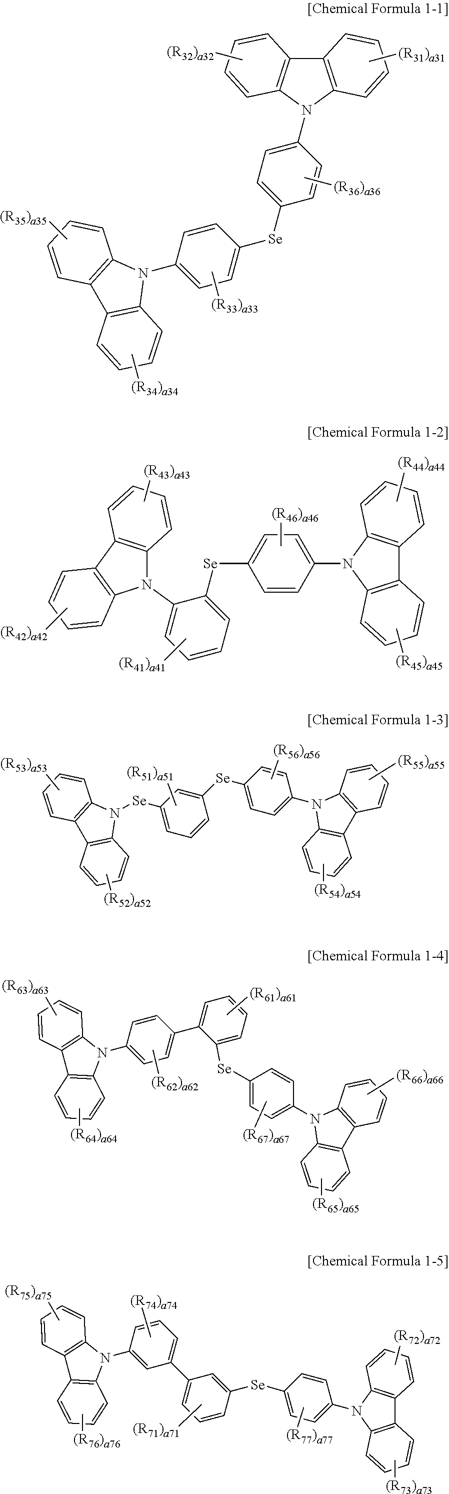

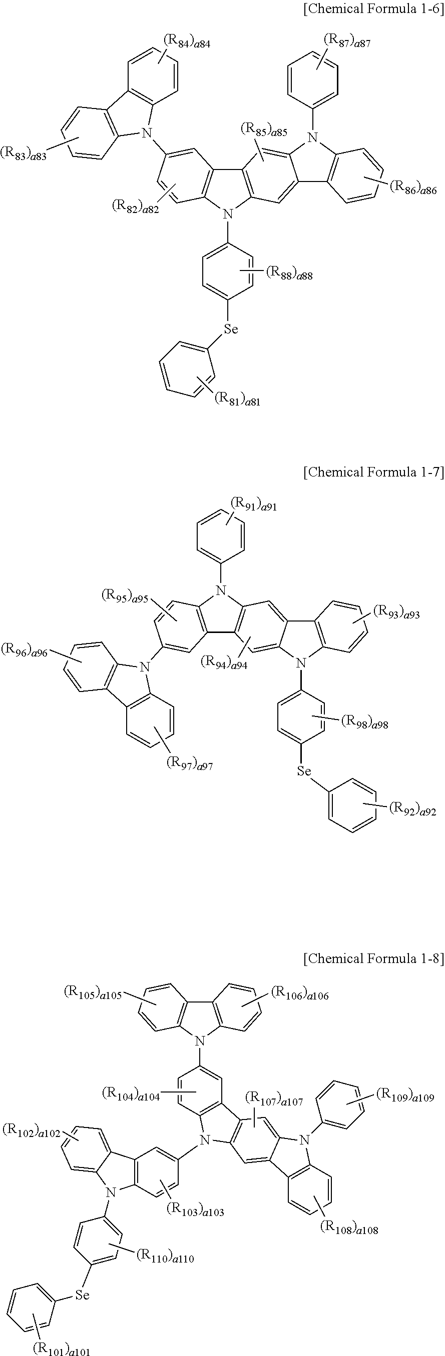

In Chemical Formulae 1-1 to 1-8, R₃₁ to R₃₆, R₄₁ to R₄₆, R₅₁ to R₅₆, R₆₁ to R₆₇, R₇₁ to R₇₇, R₈₁ to R₈₈, R₉₁ to R₉₈, and R₁₀₁ to R₁₁₀ may each independently be hydrogen, deuterium, a substituted or unsubstituted C₁-C₆₀ alkyl group, a substituted or unsubstituted C₂-C₆₀ heteroaryl group, —Se(Q₁), or —Si(Q₁)(Q₂)(Q₃). In an aspect, two or more of R₃₁ to R₃₆, R₄₁ to R₄₆, R₅₁ to R₅₆, R₆₁ to R₆₇, R₇₁ to R₇₇, R₈₁ to R₈₈, R₉₁ to R₈₈, and R₁₀₁ to R₁₁₀ may be combined with each other to form a substituted or unsubstituted C₂-C₆₀ heteroaryl ring.

a31 to a36, a41 to a46, a51 to a56, a61 to a67, a71 to a77, a81, a83, a84, a86, a93, a96 to a98, a102, a105, a106, a108, and a110 may each independently be an integer from 0 to 4.

a82, a95, a103, and a104 may each independently be an integer from 0 to 3. a85, a94, and a107 may each independently be an integer from 0 to 2. a87, a91, a92, a101, and a109 may each independently be an integer from 0 to 5.

When a31 to a36, a41 to a46, a51 to a56, a61 to a67, a71 to a77, a81 to a88, a91 to a98, and a101 to a110 are each 2 or more, multiple R₃₁ to R₃₆, R₄₁ to R₄₆, R₅₁ to R₅₆, R₆₁ to R₆₇, R₇₁ to R₇₇, R₈₁ to R₈₈, R₈₁ to R₈₈, and R₁₀₁ to R₁₁₀ may each independently be the same or different from each other.

In some aspects, R₃₁ to R₃₆, R₄₁ to R₄₆, R₅₁ to R₅₆, R₆₁ to R₆₇, R₇₁ to R₇₇, R₈₁ to R₈₈, R₉₁ to R₉₈, and R₁₀₁ to R₁₁₀ may each independently be hydrogen, deuterium, or —Si(Q₁)(Q₂)(Q₃) and Q₁ to Q₃ may be a phenyl group.

In some aspects, Chemical Formula 2 may be represented by any one of Chemical Formulae 2-1 to 2-3.

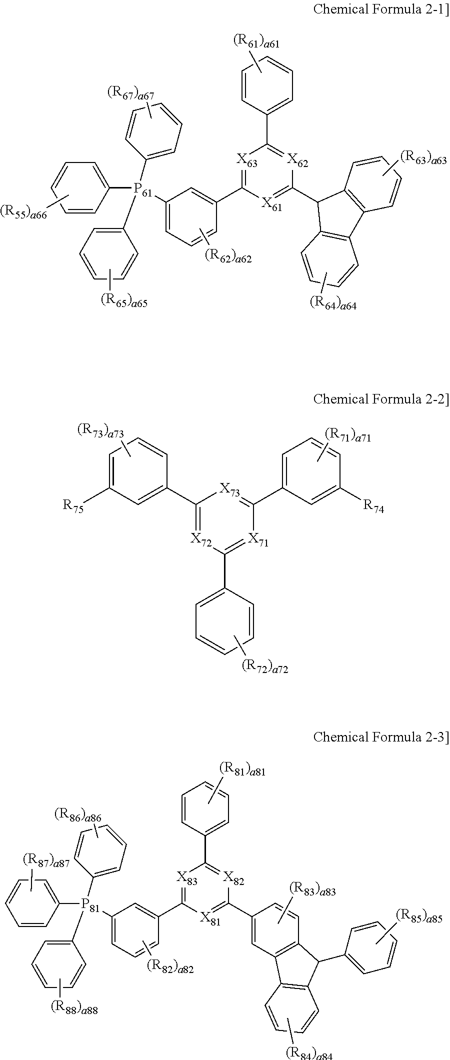

In Chemical Formulae 2-1 to 2-3, X₆₁ to X₆₃, X₇₁ to X₇₃ and X₈₁ to X₈₃ may each independently be C(R_{1b}) or N, and R_{1b} may be hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or an unsubstituted C₆-C₆₀ aryloxy group, a substituted or unsubstituted C₈-C₆₀ condensed polycyclic group, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂).

P₆₁ and P₈₁ may each independently be C, Si or Ge.

In an aspect, R₆₁ to R₆₇, R₇₁ to R₇₅, and R₈₁ to R₈₈ may each independently be hydrogen, deuterium, a substituted or unsubstituted C₁-C₆₀ alkyl group, a substituted or unsubstituted C₂-C₆₀ heteroaryl group, or —Si(Q₁)(Q₂)(Q₃). In an aspect, two or more adjacent ones of R₆₁ to R₆₇, R₇₁ to R₇₅, and R₈₁ to R₈₈ may be combined with each other to form a substituted or unsubstituted C₂-C₆₀ heteroaryl ring.

Q₁ to Q₃ may each independently be hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, or a substituted or unsubstituted C₈-C₆₀ condensed polycyclic ring.

a61, a65 to a67, a72, a81, and a85 to a88 may each independently be an integer from 0 to 5, a62 to a64, a71, a73, a82, and a84 are each independently an integer from 0 to 4, and a83 may be an integer from 0 to 3.

When a61 to a67, a71 to a73, and a81 to a88 are each 2 or more, multiple R₆₁ to R₆₇, R₇₁ to R₇₅, and R₈₁ to R₈₈ may each independently be the same or different from each other.

In an aspect, X₆₁ to X₆₃, X₇₁ to X₇₃, and X₈₁ to X₈₃ are N.

In an aspect, R₆₁ to R₆₇, R₇₁ to R₇₅, and R₈₁ to R₈₈ may each independently be hydrogen, deuterium, a substituted or unsubstituted C₁-C₆₀ alkyl group, a substituted or unsubstituted unsubstituted C₂-C₆₀ heteroaryl group, or —Si(Q₁)(Q₂)(Q₃). In an aspect, two or more adjacent ones of R₆₁ to R₆₇, R₇₁ to R₇₅, and R₈₁ to R₈₈ may be combined with each other to form a substituted or unsubstituted C₂-C₆₀ heteroaryl ring.

In an aspect, R₆₁ to R₆₇, R₇₁ to R₇₅, and R₈₁ to R₈₈ may each independently be hydrogen, deuterium, a substituted or unsubstituted C₁-C₅₀ alkyl group, a substituted or unsubstituted unsubstituted C₂-C₅₀ heteroaryl group, or —Si(Q₁)(Q₂)(Q₃). In an aspect, two or more adjacent ones of R₆₁ to R₆₇, R₇₁ to R₇₅, and R₈₁ to R₈₈ may be combined with each other to form a substituted or unsubstituted C₂-C₅₀ heteroaryl ring.

In an aspect, R₆₁ to R₆₇, R₇₁ to R₇₅, and R₈₁ to R₈₈ may each independently be hydrogen, deuterium, a substituted or unsubstituted C₁-C₄₀ alkyl group, a substituted or unsubstituted unsubstituted C₂-C₄₀ heteroaryl group, or —Si(Q₁)(Q₂)(Q₃). In an aspect, two or more adjacent ones of R₆₁ to R₆₇, R₇₁ to R₇₅, and R₈₁ to R₈₈ may be combined with each other to form a substituted or unsubstituted C₂-C₄₀ heteroaryl ring.

In an aspect, R₆₁ to R₆₇, R₇₁ to R₇₅, and R₈₁ to R₈₈ may each independently be hydrogen, deuterium, a substituted or unsubstituted C₁-C₃₀ alkyl group, a substituted or unsubstituted unsubstituted C₂-C₃₀ heteroaryl group, or —Si(Q₁)(Q₂)(Q₃). In an aspect, two or more adjacent ones of R₆₁ to R₆₇, R₇₁ to R₇₅, and R₈₁ to R₈₈ may be combined with each other to form a substituted or unsubstituted C₂-C₃₀ heteroaryl ring.

In an aspect, R₆₁ to R₆₇, R₇₁ to R₇₅, and R₈₁ to R₈₈ may each independently be hydrogen, deuterium, a substituted or unsubstituted C₁-C₂₀ alkyl group, a substituted or unsubstituted unsubstituted C₂-C₂₀ heteroaryl group, or —Si(Q₁)(Q₂)(Q₃). In an aspect, two or more adjacent ones of R₆₁ to R₆₇, R₇₁ to R₇₅, and R₈₁ to R₈₈ may be combined with each other to form a substituted or unsubstituted C₂-C₂₀ heteroaryl ring.

In an aspect, R₆₁ to R₆₇, R₇₁ to R₇₅, and R₈₁ to R₈₈ may each independently be hydrogen, deuterium, a substituted or unsubstituted methyl group, a substituted or unsubstituted carbazole group, or —Si(Q₁)(Q₂)(Q₃). In an aspect, two or more adjacent ones of R₆₁ to R₆₇, R₇₁ to R₇₅, and R₈₁ to R₈₈ may be combined with each other to form a substituted or unsubstituted carbazole ring, and Q₁ to Q₃ may each independently be a substituted or unsubstituted phenyl group.

In an aspect, R₆₁ to R₆₇, R₇₁ to R₇₅, and R₈₁ to R₈₈ may each independently be hydrogen, deuterium, a methyl group, a carbazole group, or —Si(Q₁)(Q₂)(Q₃). In an aspect, two or more adjacent ones of R₆₁ to R₆₇, R₇₁ to R₇₅, and R₈₁ to R₈₈ may be combined with each other to form a carbazole ring, and Q₁ to Q₃ may be a phenyl group.

In some aspects, at least one of R₇₁, R₇₃, R₇₄ and R₇₅ may be represented by any one of Chemical Formulae 2-2A-1 to 2-2A-4.

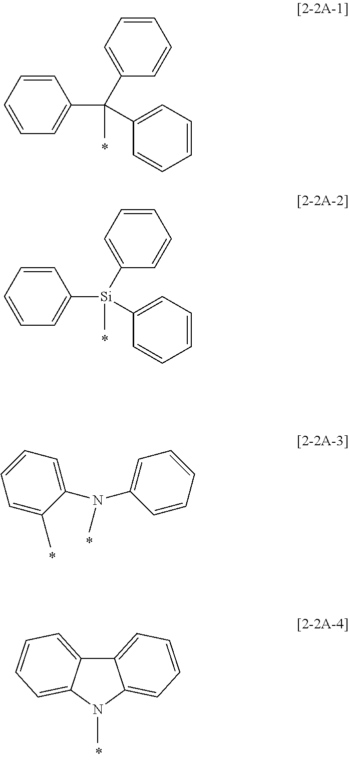

In Chemical Formulae 2-2A-1 to 2-2A-4, * indicates a bonding position.

In some aspects, R₇₄ and R₇₅ may be represented by Chemical Formulae 2-2A-1, 2-2A-2, or 2-2A-3.

In some aspects, Rₙ and R₇₄ may be represented by Chemical Formula 2-2A-3.

In some aspects, R₇₃ and R₇₅ may be represented by Chemical Formula 2-2A-3.

In some aspects, the compound represented by Chemical Formula 1 may be any one of the following compounds represented by Chemical Formulae 1-9 to 1-17.

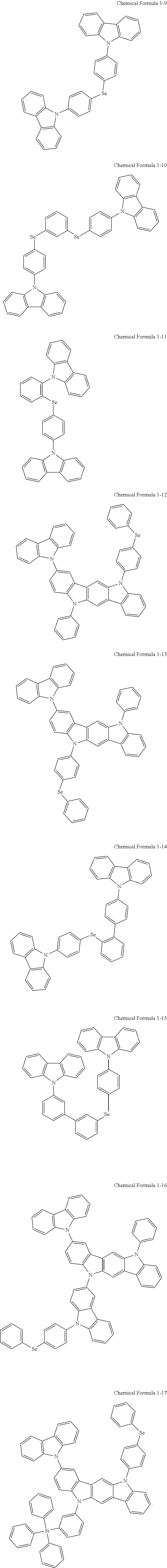

In some aspects, the compound represented by Chemical Formula 2 may be any one of the following compounds represented by Chemical Formulae 2-4 to 2-15.

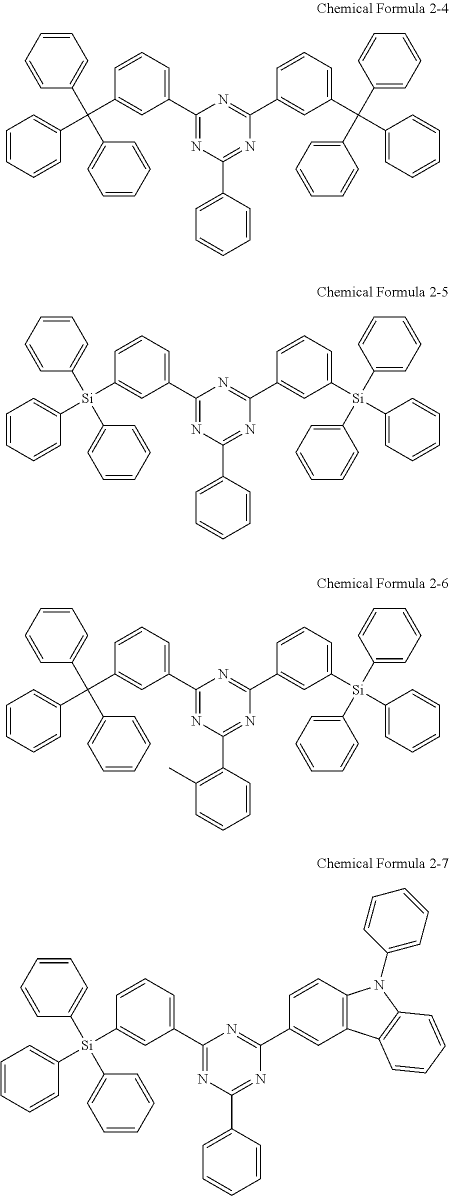 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc([Si](c3ccccc3)(c3ccccc3)c3ccccc3)c2)n1 ; c1ccc(-c2nc(-c3cccc([Si](c4ccccc4)(c4ccccc4)c4ccccc4)c3)nc(-c3ccc4c(c3)c3ccccc3n4-c3ccccc3)n2)cc1]

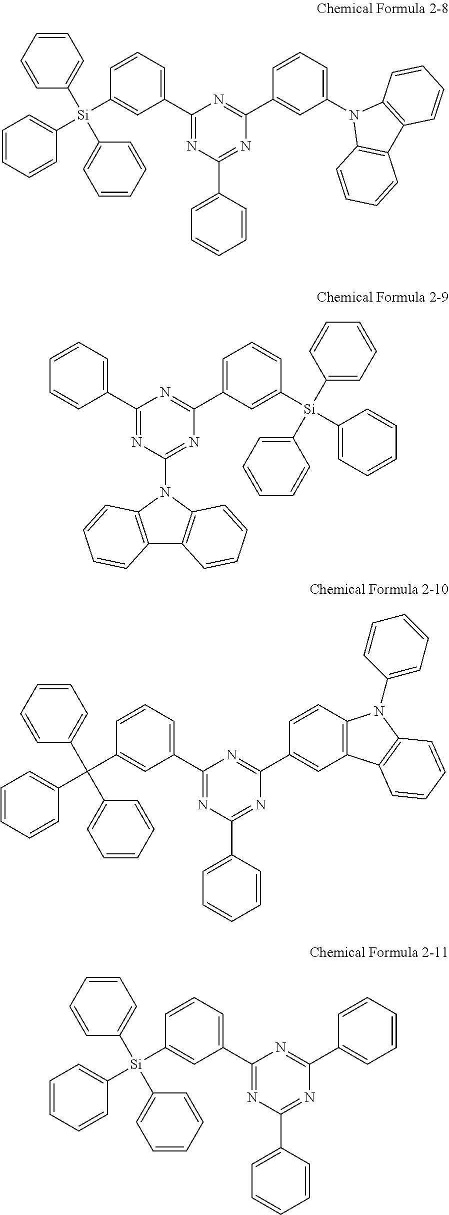 [SMILES: c1ccc(-c2nc(-c3cccc(-n4c5ccccc5c5ccccc54)c3)nc(-c3cccc([Si](c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-n3c4ccccc4c4ccccc43)n2)cc1 ; c1ccc(-c2nc(-c3cccc(C(c4ccccc4)(c4ccccc4)c4ccccc4)c3)nc(-c3ccc4c(c3)c3ccccc3n4-c3ccccc3)n2)cc1 ; c1ccc(-c2nc(-c3ccccc3)nc(-c3cccc([Si](c4ccccc4)(c4ccccc4)c4ccccc4)c3)n2)cc1]

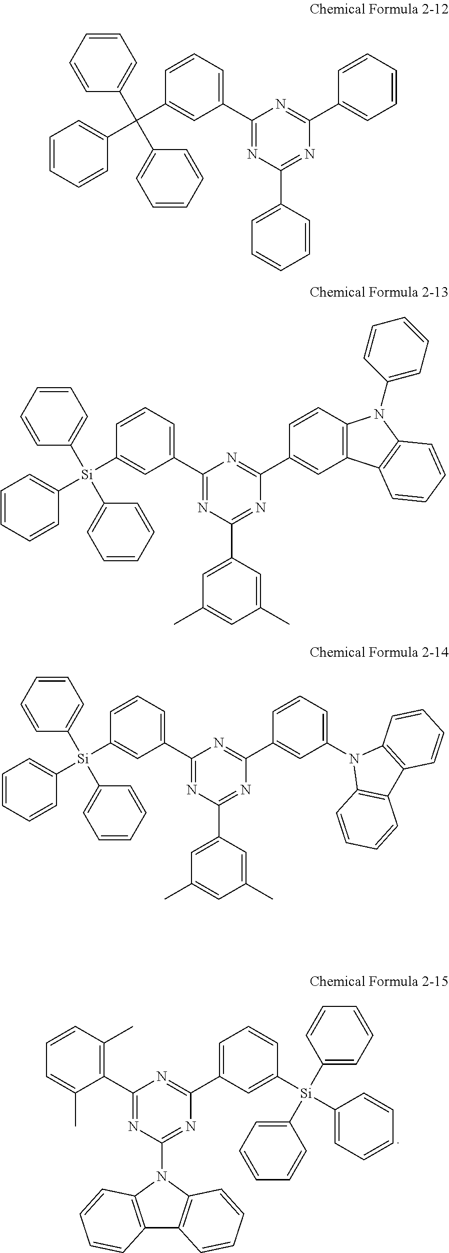 [SMILES: c1ccc(-c2nc(-c3ccccc3)nc(-c3cccc(C(c4ccccc4)(c4ccccc4)c4ccccc4)c3)n2)cc1 ; Cc1cc(C)cc(-c2nc(-c3cccc([Si](c4ccccc4)(c4ccccc4)c4ccccc4)c3)nc(-c3ccc4c(c3)c3ccccc3n4-c3ccccc3)n2)c1 ; Cc1cc(C)cc(-c2nc(-c3cccc(-n4c5ccccc5c5ccccc54)c3)nc(-c3cccc([Si](c4ccccc4)(c4ccccc4)c4ccccc4)c3)n2)c1 ; Cc1cccc(C)c1-c1nc(-c2cccc([Si](c3ccccc3)(c3ccccc3)c3ccccc3)c2)nc(-n2c3ccccc3c3ccccc32)n1 ; "."]

In some aspects, a weight ratio of the compound represented by Chemical Formula 1 and the compound represented by Chemical Formula 2 included in the composition for forming an emission layer may be from 1:9 to 9:1, from 2:8 to 8:2, from 3:7 to 7:3, or from 4:6 to 6:4.

In some aspects, the weight ratio of the compound represented by Chemical Formula 1 and the compound represented by Chemical Formula 2 may be about 5:5.

In the above range of the weight ratio, both luminous properties and life-span efficiency may be improved.

In some aspects, the emission layer may include the composition for forming the emission layer.

For example, the emission layer 130 may include a host material widely known in the related art, such as an anthracene derivative, a pyrene derivative, a fluoranthene derivative, a chrysene derivative, a dihydrobenzanthracene derivative, a triphenylene derivative, or the like.

In some aspects, the emission layer 130 may include, e.g., a host material represented by Chemical Formula FH. For example, the compound represented by Chemical Formula FH may be used as a fluorescent host material.

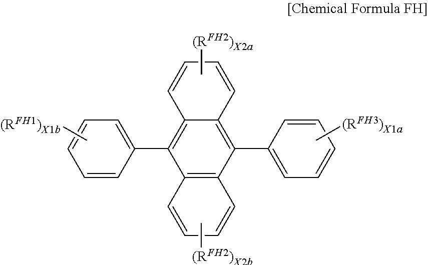

In Chemical Formula FH, R^{FH1} to R^{FH4} may each independently be a hydrogen atom, a deuterium atom, a halogen atom, a substituted or unsubstituted silyl group, a substituted or unsubstituted thio group, a substituted or unsubstituted oxy group, a substituted or unsubstituted C₁-C₁₀ alkyl group, a substituted or unsubstituted C₂-C₁₀ alkenyl group, a substituted or unsubstituted C₆-C₃₀ aryl group, a substituted or unsubstituted C₆-C₃₀ heteroaryl group, or a cyclic group formed through a combination thereof. In an aspect, in Chemical Formula FH, at least one of R^{FH1} to R^{FH4} may form a condensed ring with a bonded benzene ring.

In Chemical Formula FH, x1a and x1b may each independently be an integer from 0 to 5; and x2a and x2b may each independently be an integer from 0 to 4. When x1a, x1b, x2a, and x2b are each 2 or more, two or more of each of R^{FH1} to R^{FH4} may be the same as or different from each other.

Non-limiting examples of compounds represented by Chemical Formula FH are as follows.

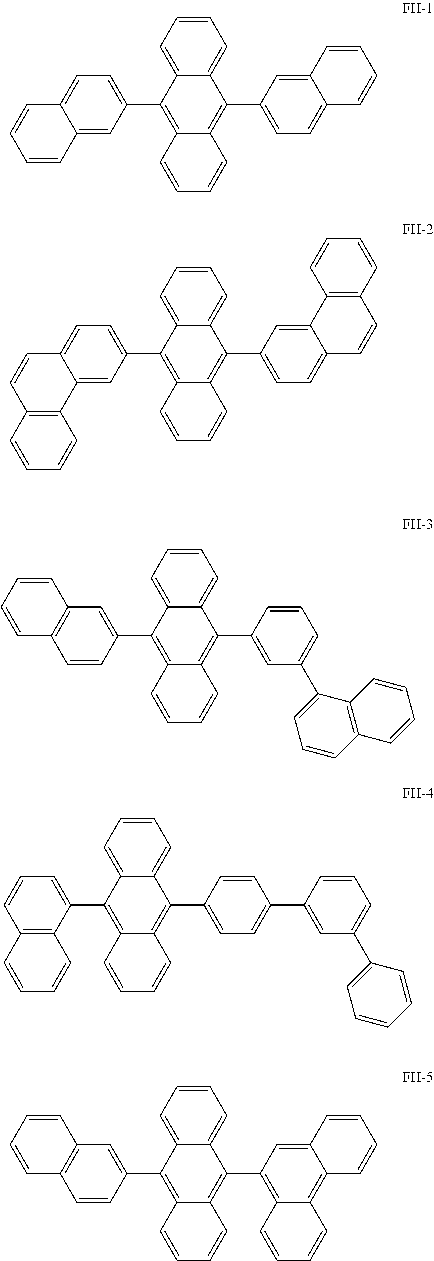

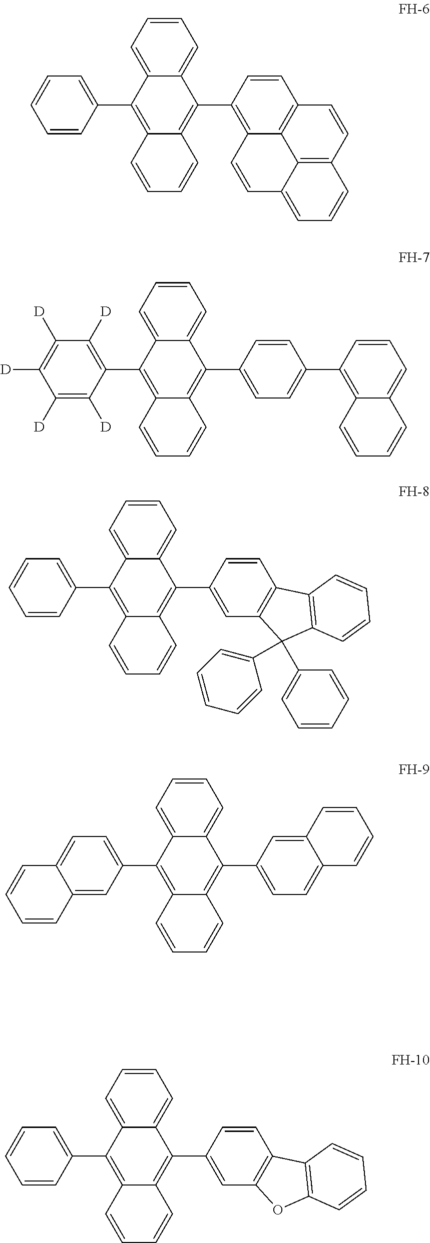

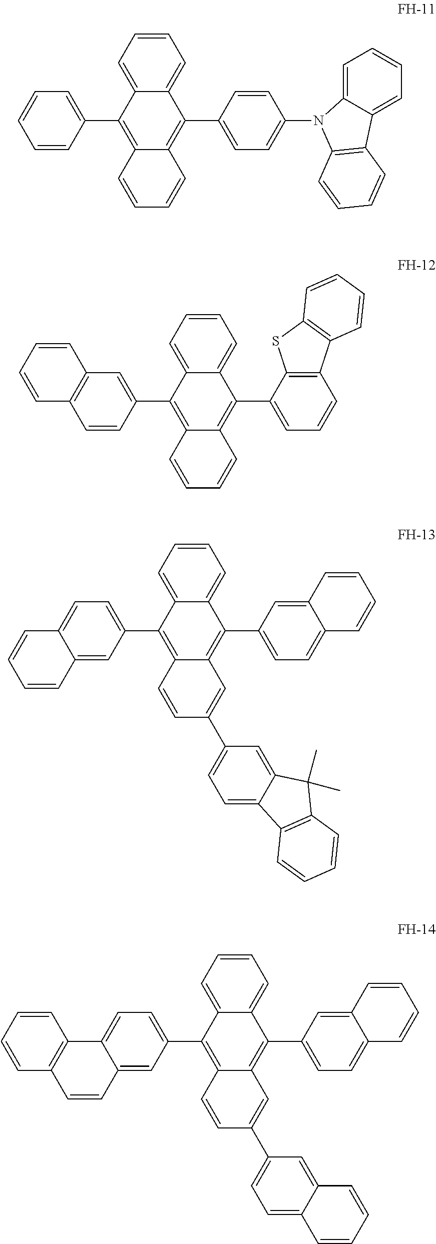

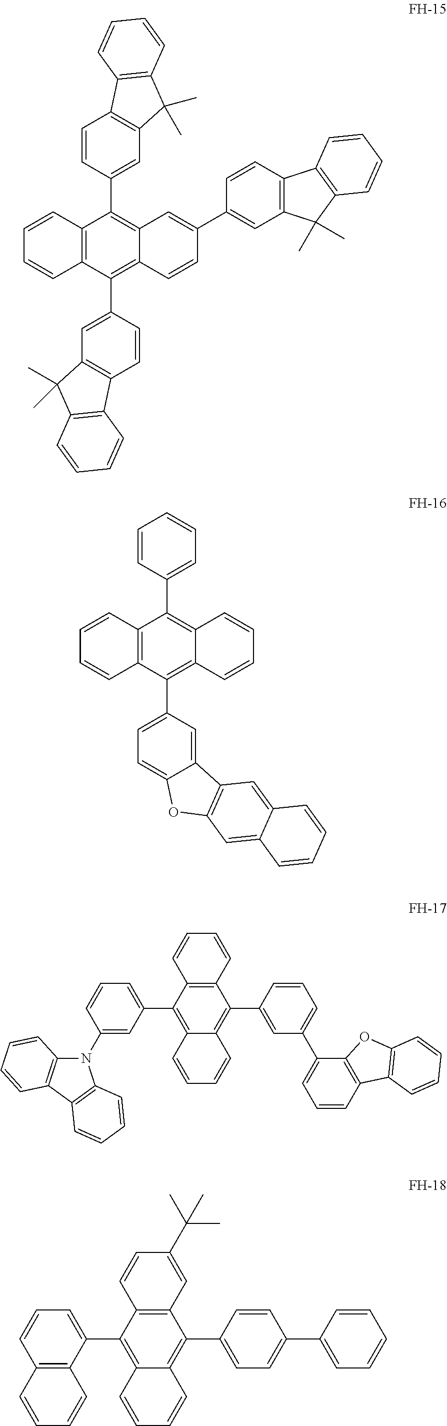

In some aspects, the emission layer 130 may include, e.g., a host material represented by Chemical Formula PH. For example, the compound represented by Chemical Formula PH may be used as a host material for a phosphorescent device.

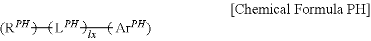

In Chemical Formula PH, R^{PH} may be a substituted or unsubstituted carbazole group. L^{PH} may be a direct linkage, a substituted or unsubstituted C₆-C₃₀ arylene group, or a substituted or unsubstituted C₂-C₃₀ heteroarylene group. Ar^{PH} may be a substituted or unsubstituted C₆-C₃₀ aryl group, or a substituted or unsubstituted C₂-C₃₀ heteroaryl group.

In Chemical Formula PH, 1× may be an integer from 0 to 10. When 1× is 2 or more, two or more of L^{PH} may be the same as or different from each other.

In an aspect, a substituent included in Ar^{PH} may be a silyl group represented by —Si(R^{sa})(R^{sb})(R^{sc}); and R^{sa}, R^{sb}, and R^{sc} may each independently be hydrogen, a halogen, a hydroxyl group, a C₁-C₆₀ alkyl group, a C₁-C₆₀ alkoxy group, a C₆-C₆₀ aryl group, or a C₂-C₃₀ heteroaryl group. At least one of R^{sa}, R^{sb}, and R^{sc} may be a C₆-C₆₀ aryl group or a C₂-C₃₀ heteroaryl group. For example, R^{sa}, R^{sb} and R^{sc} may each independently be a C₆-C₆₀ aryl group or a C₂-C₃₀ heteroaryl group.

Non-limiting examples of compounds represented by Chemical Formula PH are as follows.

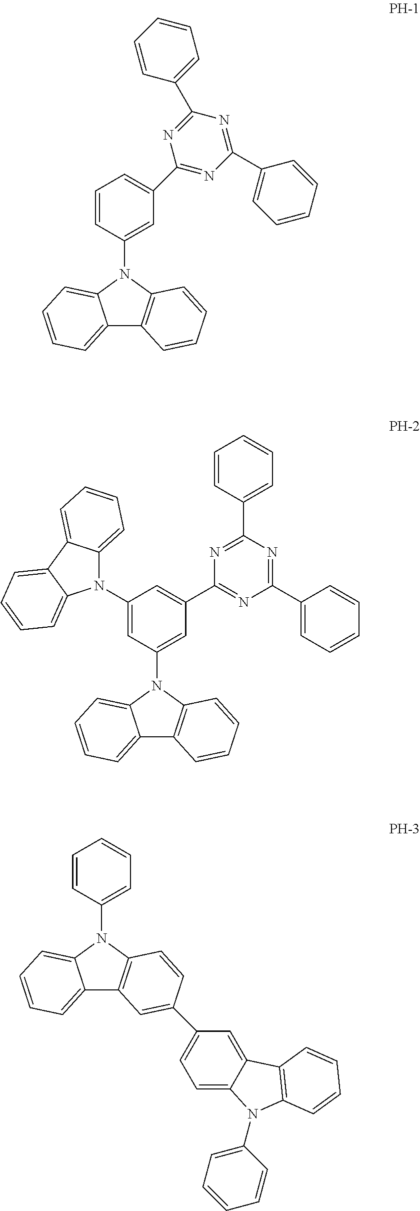 [SMILES: c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1ccc(-n2c3ccccc3c3cc(-c4ccc5c(c4)c4ccccc4n5-c4ccccc4)ccc32)cc1]

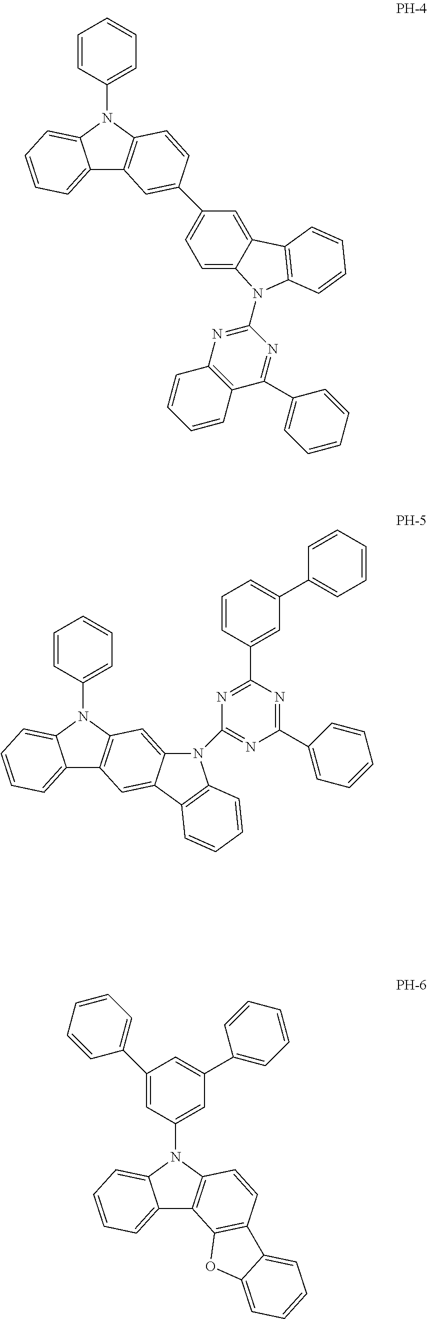 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-c3ccccc3)cc(-n3c4ccccc4c4c5oc6ccccc6c5ccc43)c2)cc1]

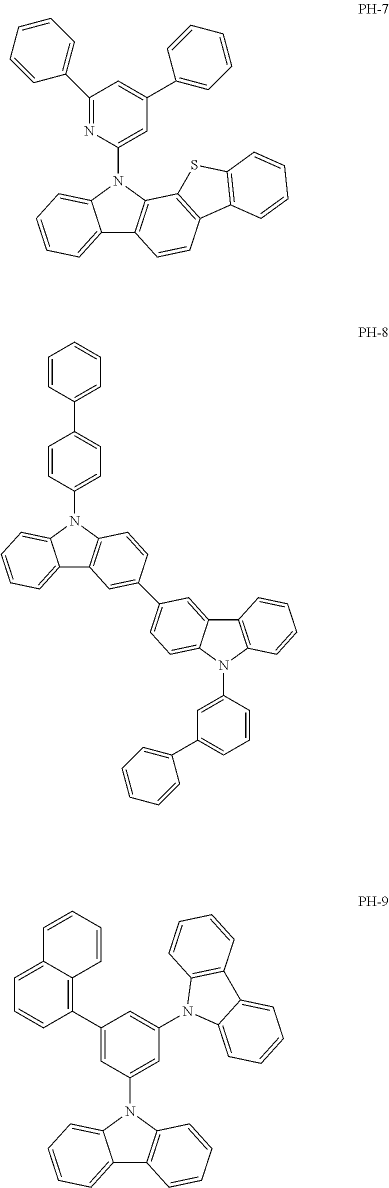 [SMILES: c1ccc(-c2cc(-c3ccccc3)nc(-n3c4ccccc4c4ccc5c6ccccc6sc5c43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

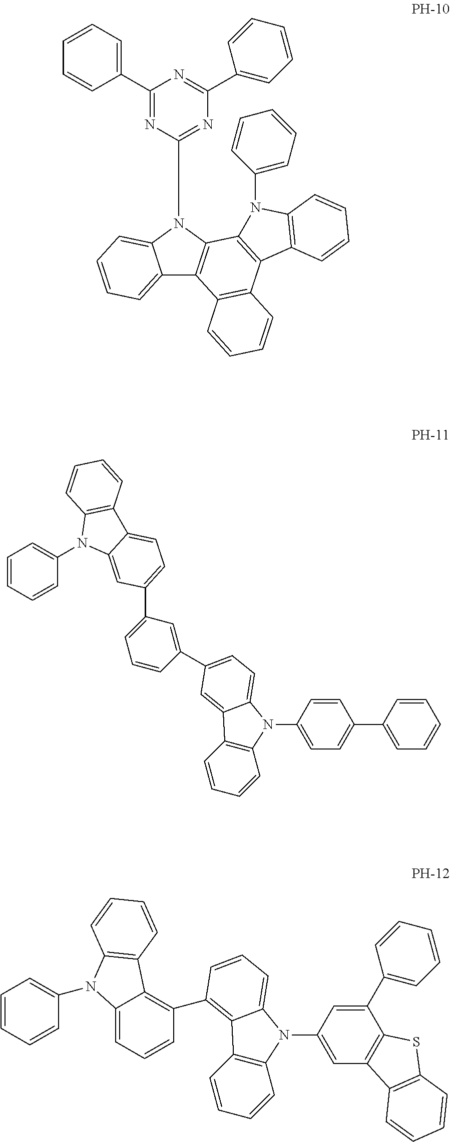 [SMILES: c1ccc(-c2nc(-c3ccccc3)nc(-n3c4ccccc4c4c5ccccc5c5c6ccccc6n(-c6ccccc6)c5c43)n2)cc1 ; c1ccc(-c2ccc(-n3c4ccccc4c4cc(-c5cccc(-c6ccc7c8ccccc8n(-c8ccccc8)c7c6)c5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

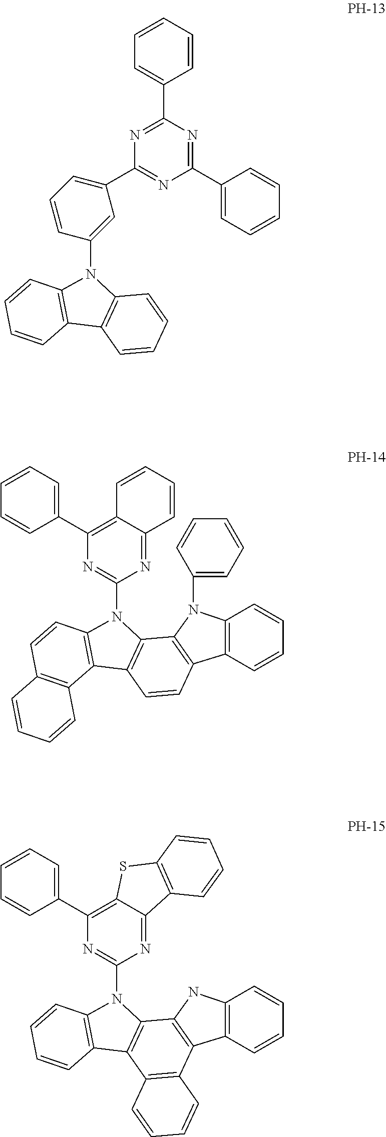 [SMILES: c1ccc(-c2nc(-c3ccccc3)nc(-c3cccc(-n4c5ccccc5c5ccccc54)c3)n2)cc1 ; c1ccc(-c2nc(-n3c4ccc5ccccc5c4c4ccc5c6ccccc6n(-c6ccccc6)c5c43)nc3ccccc23)cc1 ; c1ccc(-c2nc(-n3c4ccccc4c4c5ccccc5c5c6ccccc6[nH]c5c43)nc3c2sc2ccccc23)cc1]

The emission layer 130 may include, e.g., BCPDS (bis(4-(9H-carbazol-9-yl) phenyl) diphenylsilane), POPCPA ((4-(1-(4-(diphenylamino) phenyl) cyclohexyl) phenyl) diphenyl-phosphine oxide), DPEPO (bis[2-(diphenylphosphino)phenyl]ether oxide), mCBP (3,3′-di(9H-carbazol-9-yl)-1,1′-biphenyl), CBP (4,4′-bis(N-carbazolyl)-1,1′-biphenyl), mCP (1,3-bis(carbazol-9-yl)benzene), PPF (2,8-bis(diphenylphosphoryl) dibenzo[b,d]furan), TCTA (4,4′,4″-tris(carbazol-9-yl)-triphenylamine), TPBi (1,3,5-tris(1-phenyl-1H-benzo[d]imidazole-2-yl)benzene), Alq3 (tris(8-hydroxyquinolino) aluminum), ADN (9,10-di(naphthalene-2-yl)anthracene), TBADN (2-tert-butyl-9,10-di(naphth-2-yl)anthracene), DSA (distyrylarylene), CDBP (4,4′-bis(9-carbazolyl)-2,2′-dimethyl-biphenyl), MADN (2-methyl-9,10-bis(naphthalen-2-yl)anthracene), CP1 (hexaphenyl cyclotriphosphazene), UGH2 (1,4-bis(triphenylsilyl)benzene), DPSiO3 (hexaphenylcyclotrisiloxane), DPSiO4 (octaphenylcyclotetrasiloxane), etc., as a host material.

In an aspect, in the emission layer 130, the host may include one of the materials as described above, or any combination thereof.

The emission layer 130 may further include a dopant interacting with the host.

In some aspects, the emission layer 130 may include a dopant represented by Chemical Formula FD. For example, the compound represented by Chemical Formula FD may be used as a fluorescent dopant.

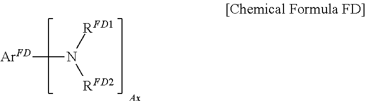

In Chemical Formula FD, Ar^{FD}, R^{FD1}, and R^{FD2} may each independently be a substituted or unsubstituted C₃-C₆₀ carbocyclic group, or a substituted or unsubstituted C₁-C₆₀ heterocyclic group. Ax may be an integer from 1 to 6.

In some aspects, Ar^{FD} may include a condensed ring structure in which three or more aryl rings or benzene rings are condensed together (e.g., an anthracene group, a chrysene group, a pyrene group, etc.).

Non-limiting examples of compounds represented by Chemical Formula FD are as follows.

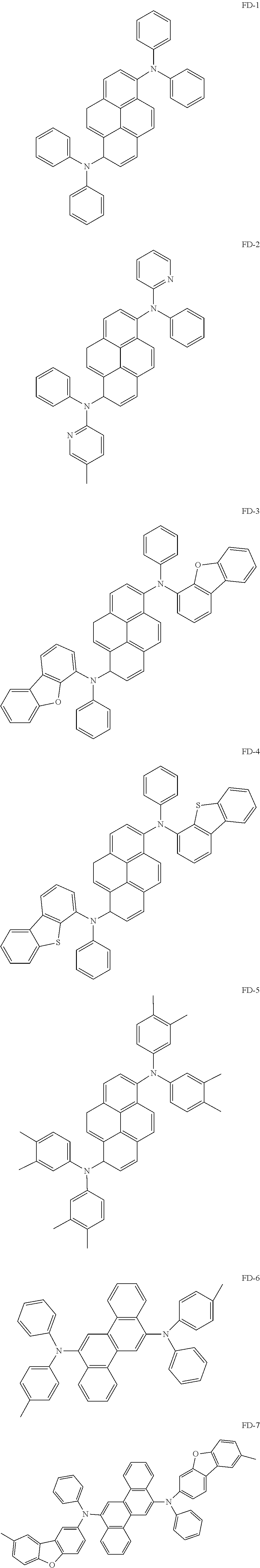

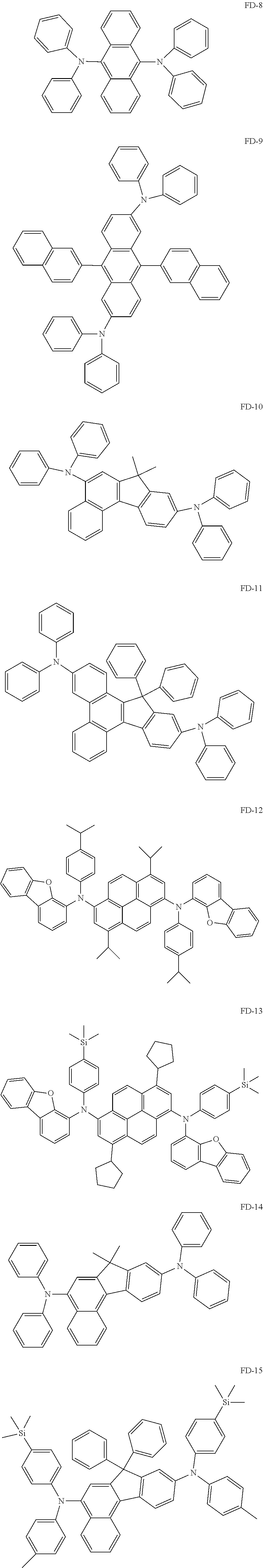

In some aspects, the emission layer 130 may include a dopant for a phosphorescent device. The dopant for the phosphorescent device may include an organometallic compound that includes a central metal and at least one ligand bonded to the central metal via a coordination bond. The central metal may include, e.g., a transition metal, and the ligand may include, e.g., a monodentate ligand, a bidentate ligand, a tridentate ligand, a tetradentate ligand, a pentadentate ligand, a hexadentate ligand, or a combination thereof.

The dopant for the phosphorescent device may include, e.g., a compound represented by Chemical Formula PD.

M(L_{d}¹)_{dx1}(L_{d}²)_{dx2} [Chemical Formula PD]

In Chemical Formula PD, M may be a transition metal atom, e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), ruthenium (Ru), copper (Cu), or thulium (Tm).

In Chemical Formula PD, L_{d}¹ may be a ligand represented by Chemical Formula LD1.

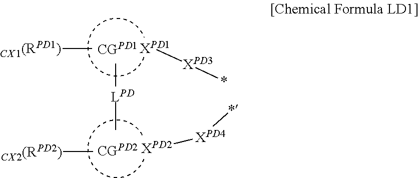

In Chemical Formula LD1, X^{PD1} and X^{PD2} may each independently be C or N.

In an aspect, one of X^{PD1} and X^{PD2} may be C and the other may be N. In an aspect, X^{PD1} and X^{PD2} may each be N.

In Chemical Formula LD1, CG^{PD1} and CG^{PD2} may each independently be a substituted or unsubstituted C₃-C₆₀ carbocyclic group, or a substituted or unsubstituted C₁-C⁶⁰ heterocyclic group.

For example, CG^{PD1} and CG^{PD2} may each independently be a pyrrole group, a pyrazole group, an imidazole group, a triazole group, an oxazole group, an isoxazole group, a thiazole group, an isothiazole group, an oxadiazole group or a thiadiazole group, a benzene group, a pyridine group, a pyrimidine group, a naphthalene group, a dibenzofuran group, a dibenzothiophene group, a carbazole group, a fluorene group, a dibenzosilole group, a naphthobenzofuran group, a naphthobenzothiophene group, a benzocarbazole group, a benzofluorene group, a naphthobenzosilole group, a dinaphthofuran group, a dinaphthothiophene group, a dibenzocarbazole group, a dibenzofluorene group, a dinaphthosilole group, an azadibenzofuran group, an azadibenzothiophene group, an azacarbazole group, an azafluorene group, an azadibenzosilole group, an azanaphthobenzofuran group, an azanaphthobenzothiophene group, an azabenzocarbazole group, an azabenzofluorene group, an azanaphthobenzosilole group, an azadinapthofuran group, an azadinapthothiophene group, an azadibenzocarbazole group, an azadibenzofluorene group, or an azadinapthosilole group.

In Chemical Formula LD1, L^{PD} may be a single bond, a substituted or unsubstituted methylene group, a substituted or unsubstituted ethylene group, *—O—*′, *—S—*′, *—C(═O)—*′, *—N(R^{PD3})—*′, *—C(R^{PD4})═*′, or *═C(R^{PD5})—*′.

In Chemical Formula LD1, X^{PD3} and X^{PD4} may each independently be a chemical bond, O, S, N(R^{PD6}), B(R^{PD7}), P(R^{PD8}), C(R^{PD8})(R^{PD9}), or Si(R^{PD10})(R^{PD11}). The chemical bond may be, e.g., a covalent bond or a coordination bond.

In Chemical Formula LD1, R^{PD1} and R^{PD2} may each independently be hydrogen, deuterium, —F, —Cl, —Br, —I, —OH, —CN, —NO₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, a substituted or unsubstituted C₈-C₆₀ condensed polycyclic group, a substituted or unsubstituted silyl group, a substituted or unsubstituted amine group, a substituted or unsubstituted aniline group, —B(R^{PD12})(R^{PD13}), —C(═O)(R^{PD14}), —S(═O)₂(R^{PD15}), or —P(═O)(R^{PD16})(R^{PD17}). The silyl group may be represented by —Si(R^{sa})(R^{sb}) (R^{sc}), as explained above.

R^{PD3} to R^{PD17} may each independently be hydrogen, deuterium, —F, —Cl, —Br, —I, —OH, —CN, —NO₂, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, or a substituted or unsubstituted C₈-C₆₀ condensed polycyclic group.

In Chemical Formula LD1, cx1 and cx2 may each independently be an integer from 0 to 10. When at least one of cx1 and cx2 is 2 or more, two or more of R^{PD1} or two or more of R^{PD2} may be the same as or different from each other.

The symbols -* and -*′ each represent a binding site where the ligand represented by Chemical Formula LD1 bonds to M.

In Chemical Formula PD, dx1 may be an integer from 1 to 3. When dx1 is 2 or 3, two or three of L_{d}¹ may be the same as or different from each other. Among two or three of L_{d}¹, CG^{PD1} and/or CG^{PD2} adjacent to each other may be connected to each other through a connecting group such as L^{PD1}, L^{PD2}, etc. The connecting group such as L^{PD1}, L^{PD2}, etc., may each independently be the same as defined in connection with L^{PD}.

In Chemical Formula PD, L_{d}² may be an organic ligand. L_{d}² may include, e.g., a halogen group, CO, NO, CS, picolinate, acetate, oxalate, a diketone group, an isonitrile group, isothiocyanato-N, thiosulphato-S, an alkyl phosphine, phenylphosphine, an aryl phosphine, phosphine oxide, phosphite, or a combination thereof.

In Chemical Formula PD, dx2 is an integer of 1 to 4. When dx2 is 2 or more, two or more of L_{d}² may be the same as or different from each other.

In some aspects, the emission layer 130 may include a styryl derivative (e.g., 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (NBDAVBi), etc.), 4,4′-bis[2-(4-(N,N-diphenylamino)phenyl)vinyl]biphenyl (DPAVBi), perylene or a derivative thereof (e.g., 2,5,8,11-tetra-t-butylperylene (TBP)), pyrene or a derivative thereof (e.g., 1,1-dipyrene, 1,4-dipyrenylbenzene, 1,4-bis(N,N-diphenylamino)pyrene), etc.), etc., as a fluorescent dopant material.

The emission layer 130 may include a metal complex that includes iridium (Ir), platinum (Pt), osmium (Os), gold (Au), titanium (Ti), zirconium (Zr), hafnium (Hf), europium (Eu), terbium (Tb), or thulium (Tm) as a phosphorescent dopant, in addition to the materials described above. For example, FIrpic (iridium(III) bis(4,6-difluorophenylpyridinato-N,C2′)picolinate), FIr6 (bis(2,4-difluorophenylpyridinato)-tetrakis(1-pyrazolyl)borate iridium(III)), PtOEP (platinum octaethyl porphyrin), etc., may be used as a phosphorescent dopant.

In aspects, the emission layer 130 may include a boron-containing dopant represented by Chemical Formula BD.

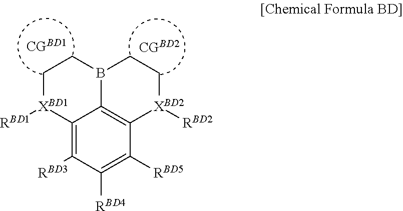

In Chemical Formula BD, X^{BD1} and X^{BD2} may each independently be N, S, O, or C. In an aspect, X^{BD1} and X^{BD2} may each be N. R^{BD1} and R^{BD2} may each independently be hydrogen, deuterium, a substituted or unsubstituted C₁-C₂₀ alkyl group, a substituted or unsubstituted C₆-C₃₀ aryl group, or a substituted or unsubstituted C₂-C₃₀ heteroaryl group. R^{BD3}, R^{BD4}, and R^{BD5} may each independently be hydrogen, deuterium, a halogen, a cyano group, a substituted or unsubstituted amine group, a substituted or unsubstituted boryl group, a substituted or unsubstituted oxy group, a substituted or unsubstituted thio group, a substituted or unsubstituted C₁-C₂₀ alkyl group, a substituted or unsubstituted C₆-C₃₀ aryl group, or a substituted or unsubstituted C₂-C₃₀ heteroaryl group. R^{BD3}, R^{BD4} and/or R^{BD5} may be bonded to an adjacent group to form a ring.

In Chemical Formula BD, CG^{BD1} and CG^{BD2} represent a cyclic group, and CG^{BD1} and CG^{BD2} may each independently be a substituted or unsubstituted C₃-C₆₀ carbocyclic group, or a substituted or unsubstituted C₁-C₆₀ heterocyclic group. In some aspects, CG^{BD1} and CG^{BD2} may each independently be a substituted or unsubstituted C₆-C₃₀ aryl group, or a substituted or unsubstituted C₂-C₃₀ heteroaryl group.

In an aspect, CG^{BD1} and CG^{BD2} may each independently be a substituted or unsubstituted benzene ring. In this case, the boron-containing dopant may serve as a thermally activated delayed fluorescence (TADF) dopant.

In an aspect, one of CG^{BD1} and CG^{BD2} may be a non-condensed aryl group or a non-condensed heteroaryl group, and the other one thereof may be a condensed polycyclic aryl group or a condensed polycyclic heteroaryl group. In this case, the boron-containing dopant may serve as a fluorescent dopant.

In an aspect, the emission layer 130 may include one of the dopant materials as described above, or any combination thereof.

In some aspects, the emission layer 130 may include two or more host materials. For example, the emission layer 130 may include a hole transporting host and an electron transporting host. In this case, the emission layer 130 may include a hole transporting host, an electron transporting host, a photosensitive agent, and a dopant. In aspects, the hole transporting host and the electron transporting host may form an exciplex, and energy may be transferred from the exciplex to the photosensitive agent and from the photosensitive agent to the dopant, thereby inducing a light emission.

Non-limiting examples of the hole transporting host may include a compound represented by Chemical Formula HT as described below. Non-limiting examples of the electron transporting host may include a compound represented by Chemical Formula ET as described below.

In some aspects, the emission layer 130 may include quantum dots. A quantum dot may include a Group II-VI compound, a Group III-VI compound, a Group I-III-VI compound, a Group III-V group compound, a Group III-II-V group compound, a Group IV-VI compound, a Group IV element, a Group IV compound, or a combination thereof.

The quantum dot may include a core that includes the compound as described above, and a shell surrounding the core. The shell may include an inorganic oxide or a semiconductor compound. Examples of the semiconductor compound may include CdS, CdSe, CdTe, ZnS, ZnSe, ZnTe, ZnSe, ZnTeS, GaAs, GaP, GaSb, HgS, HgSe, HgTe, InAs, InP, InGaP, InSb, AlAs, AlP, AlSb, etc.

In aspects, a color of light from a quantum dot may be adjusted according to a particle size of the quantum dot. The quantum dot may be a blue quantum dot, a red quantum dot, or a green quantum dot.

### Hole Transfer Region

The hole transfer region 120 may be formed between the first electrode 110 and the emission layer 130. The hole transfer region 120 may have a single-layered or a multi-layered including different materials.

The hole transfer region 120 may include a hole injection layer, a hole transport layer, and/or an electron blocking layer, and may further include an auxiliary emission layer.

In some aspects, as illustrated in FIG. 2, the hole transfer region 120 may include a hole injection layer 122 and a hole transport layer 124, sequentially stacked from the first electrode 110.

In some aspects, as illustrated in FIG. 3, the hole transfer region 120 may include a hole injection layer 122, a hole transport layer 124, and an electron blocking layer 126, sequentially stacked from the first electrode 110. The electron blocking layer 126 may block electrons from the electron transfer region 140 to the hole transfer region 120. Accordingly, the generation of excitons in the emission layer 130 may be increased, and light-emission efficiency may be further increased.

For example, the hole transfer region 120 may include a compound represented by Chemical Formula HT.

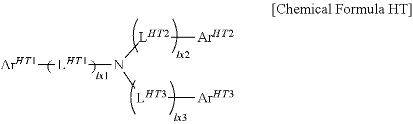

In Chemical Formula HT, L^{HT1}, L^{HT2}, and L^{HT3} may each independently be a direct linkage, a substituted or unsubstituted C₆-C₃₀ arylene group, or a substituted or unsubstituted C₂-C₃₀ heteroarylene group.

In Chemical Formula HT, 1×1 to 1×3 may each independently be an integer from 0 to 10. When 1×1, 1×2, or 1×3 is 2 or more, two or more of each of L^{HT3}, L^{HT1}, or L^{HT2}, respectively, may be directly connected by, e.g., carbon atoms (e.g., sp2 carbons) of each aryl ring, to form a substituted or unsubstituted C₆-C₃₀ arylene group, or a substituted or unsubstituted C₂-C₃₀ heteroarylene group.

In Chemical Formula HT, Ar^{HT1} and Ar^{HT2} may each independently be a substituted or unsubstituted C₆-C₃₀ aryl group, or a substituted or unsubstituted C₂-C₃₀ heteroaryl group. Ar^{HT3} may be a substituted or unsubstituted C₆-C₃₀ aryl group.

In an aspect, the compound represented by Chemical Formula HT may be a monoamine compound. In an aspect, the compound represented by Chemical Formula HT may be a diamine compound in which at least one of Ar^{HT1} to Ar^{HT3} includes an amine group as a substituent.

In some aspects, the compound represented by Chemical Formula HT may be a carbazole-based compound in which at least one of Ar^{HT1} and Ar^{HT2} includes a substituted or unsubstituted carbazole group. or a fluorene-based compound in which at least one of Ar^{HT1} and Ar^{HT2} includes a substituted or unsubstituted fluorene group.

In some aspects, two adjacent groups among Ar^{HT1} to Ar^{HT3} may be condensed together to form a ring.

For example, the hole transfer region 120 may include m-MTDATA (4,4′,4″-[tris(3-methylphenyl)phenylamino]triphenylamine), TDATA (4,4′4″-tris(N,N-diphenylamino)triphenylamine), 2-TNATA (4,4′,4″-tris[N(2-naphthyl)-N-phenylamino]-triphenylamine), NPB (N,N′-di(naphthalene-1-yl)-N,N′-diphenyl-benzidine), TPD (N,N′-bis(3-methylphenyl)-N,N′-diphenyl-[1,1′-biphenyl]-4,4′-diamine), Spiro-TPD, Spiro-NPB, DNTPD (N¹,N¹′-([1,1′-biphenyl]-4,4′-diyl)bis(N1-phenyl-N⁴,N⁴-di-m-tolylbenzene-1,4-diamine), TAPC (4,4′-cyclohexylidene bis[N,N-bis(4-methylphenyl)benzenamine]), HMTPD (4,4′-bis[N,N′-(3-tolyl)amino]-3,3′-dimethylbiphenyl), TCTA (4,4′,4″-tris(N-carbazolyl) triphenylamine), PANI/DBSA (polyaniline/dodecylbenzenesulfonic acid), PEDOT/PSS (poly(3,4-ethylenedioxythiophene)/poly(4-styrenesulfonate)) PANI/CSA (Polyaniline/Camphor sulfonicacid), PANI/PSS (polyaniline/poly(4-styrenesulfonate)), a phthalocyanine compound, a carbazole compound (N-phenylcarbazole, polyvinylcarbazole, etc.), a fluorene compound, etc. The hole transfer region 120 may include one of the hole transfer materials described above, or a combination thereof.

The hole transfer materials described above may be included in at least one of the hole injection layer 122, the hole transport layer 124, and the electron blocking layer 126.

The hole transfer region 120 may further include a charge generating material. The charge generating material may be a dopant material such as a p-dopant, so that conductivity of the hole transfer region 120 may be improved.

Examples of dopant materials may include a halogenated metal compound such as LiF, NaCl, CsF, RbCl, RbI, CuI, and KI; a quinone derivative such as TCNQ (tetracyanoquinodimethane), F4-TCNQ (2,3,5,6-tetrafluoro-7,7,8,8-tetracyanoquinodimethane), etc.; a cyano-containing compound such as HATCN (dipyrazino[2,3-f: 2′,3′-h]quinoxaline-2,3,6,7,10,11-hexacarbonitrile), NDP9 (4-[[2,3-bis[cyano-(4-cyano-2,3,5,6-tetrafluorophenyl)methylidene]cyclopropylidene]-cyanomethyl]-2,3,5,6-tetrafluorobenzonitrile), etc.; a tungsten (W) oxide; a molybdenum (Mo) oxide; etc. The hole transfer region 120 may include one of the dopant materials described above, or a combination thereof.

A thickness of the hole transfer region 120 may be in a range of about 100 Å to about 10,000 Å. For example, the thickness of the hole transfer region 120 may be in a range of about 100 Å to about 1,500 Å.

When the hole transfer region 120 includes the hole injection layer 122 or the hole transport layer 124, a thickness of the hole injection layer 122 may be in a range from about 100 Å to about 9,000 Å, from about 100 Å to about 3,000 Å, or from about 100 Å to about 1,000 Å. A thickness of the hole transport layer 124 may be in a range from 50 Å to about 2,000 Å, from about 100 Å to about 1,500 Å, from about 100 Å to about 1,000 Å, or from about 100 Å to about 600 Å.

In the thickness ranges described above, hole transfer properties may be enhanced even at a low voltage operation, and a life-span of the device may be further improved.

Each layer of the hole transfer region 120 may be formed by a process such as a vacuum deposition, a spin coating, an inkjet printing, a laser printing, a casting, a laser thermal transfer, etc.

### Electron Transfer Region

The electron transfer region 140 between the second electrode 150 and the emission layer 130. The electron transfer region 140 may have a single-layered, or a multi-layered structure including different materials.

The electron transfer region 140 may include an electron injection layer, an electron transport layer, and/or a hole blocking layer, and may further include an auxiliary emission layer.

In aspects, as illustrated in FIG. 2, the electron transfer region 140 may include an electron injection layer 142 and an electron transport layer 144, stacked from the second electrode 150 to the emission layer 130.

In some aspects, as illustrated in FIG. 3, the electron transfer region 140 may include an electron injection layer 142, an electron transport layer 144, and a hole blocking layer 146, stacked from the second electrode 150 to the emission layer 130. The hole blocking layer 146 may block or suppress holes from the hole transfer region 120. Accordingly, emission energy and luminescence efficiency in the emission layer 130 may be further improved.

For example, the hole transfer region 140 may include a compound represented by Chemical Formula ET.

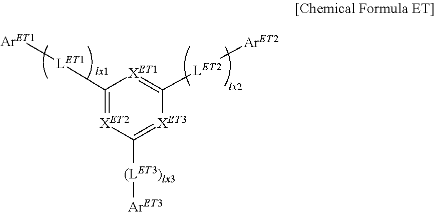

In Chemical Formula ET, at least one of X^{ET1} to X^{ET3} may be N; and the remainder of X^{ET1} to X^{ET3} may each independently be C(R^{ET}) R^{ET} may be a hydrogen atom, a deuterium atom, a substituted or unsubstituted C₁-C₂₀ alkyl group, a substituted or unsubstituted C₆-C₆₀ aryl group, or a substituted or unsubstituted C₂-C₆₀ heteroaryl group.

When one of X^{ET1} to X^{ET3} is N, the compound represented by Chemical Formula ET may include a pyridine group. When two of X^{ET1} to X^{ET3} are N, the compound represented by Chemical Formula ET may include a pyrimidine group. When X^{ET1} to X^{ET3} are each N, the compound represented by Chemical Formula ET may include a triazine group.

In Chemical Formula ET, 1×1 to 1×3 may each independently be an integer from 0 to 10. L^{ET1} to L^{ET3} may each independently be a direct linkage, a substituted or unsubstituted C₆-C₃₀ arylene group, or a substituted or unsubstituted C₂-C₃₀ heteroarylene group.

When 1×1, 1×2, or 1×3 is 2 or more, two or more of each of L^{ET1}, L^{ET2}, or L^{ET3}, respectively, may be directly linked together, e.g., by carbon atoms of each aryl ring (e.g., sp2 carbons), to form a substituted or unsubstituted C₆-C₃₀ arylene group, or a substituted or unsubstituted C₂-C₃₀ heteroarylene group.

In Chemical Formula ET, Ar^{ET1} to Ar^{ET3} may each independently be a hydrogen atom, a deuterium atom, a substituted or unsubstituted C₁-C₂₀ alkyl group, a substituted or unsubstituted C₆-C₃₀ aryl group, or a substituted or unsubstituted C₂-C₃₀ heteroaryl group. For example, Ar^{ET1} to Ar^{ET3} may each independently be a substituted or unsubstituted phenyl group, a substituted or unsubstituted carbazole group, a substituted or unsubstituted fluorene group, or a substituted or unsubstituted silyl group. The silyl group may be represented by —Si(R^{sa})(R^{sb}) (R^{sc}), as explained above.

For example, the electron transfer region 140 may include an anthracene compound, Alq3 (tris(8-hydroxyquinolinato) aluminum), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, TPBi (1,3,5-tri(1-phenyl-1H-benzo[d]imidazol-2-yl)benzene), BCP (2,9-dimethyl-4,7-diphenyl-1,10-phenanthroline), Bphen (4,7-diphenyl-1,10-phenanthroline), TAZ (3-(4-biphenylyl)-4-phenyl-5-tert-butylphenyl-1,2,4-triazole), NTAZ (4-(naphthalen-1-yl)-3,5-diphenyl-4H-1,2,4-triazole), tBu-PBD (2-(4-biphenylyl)-5-(4-tert-butylphenyl)-1,3,4-oxadiazole), BAlq (bis(2-methyl-8-quinolinolato-N1,O8)-(1,1′-biphenyl-4-olato)aluminum), Bebq₂ (beryllium bis(benzoquinolin-10-olate)), ADN (9,10-di(naphthalene-2-yl)anthracene), BmPyPhB (1,3-bis[3,5-di(pyridin-3-yl)phenyl]benzene), etc. The electron transfer region 140 may include one of the electron transfer materials described above, or a combination thereof.

The above-mentioned material may be included in at least one of the electron injection layer 142, the electron transport layer 144, and the hole blocking layer 146.

The electron transfer region 140 may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or a combination thereof. In an aspect, the above-mentioned material may be included electron injection layer 142.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include an oxide, a halide (e.g., a fluoride, a chloride, a bromide, an iodide, etc.), a telluride, or a combination thereof of the alkali metal, the alkaline earth metal, and the rare earth metal, respectively.

The alkali metal complex, the alkaline earth metal complex, and the rare earth metal complex may include a metal ion such as an alkali metal ion, an alkaline earth metal ion or a rare earth metal ion, and a ligand bonded to the metal ion. The ligand may include, e.g., hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzoimidazole, hydroxyphenylbenzothiazole, bipyridine, phenanthroline, cyclopentadiene, or a combination thereof.

A thickness of the electron transfer region 140 may be in a range from about 100 Å to about 1,000 Å, e.g., from about 150 Å to about 500 Å.

When the electron transfer region 140 includes an electron injection layer 142 or an electron transport layer 144, a thickness of the electron injection layer 142 may be in a range from about 1 Å to about 100 Å, from 1 Å to about 90 Å or from about 5 Å to about 50 Å, and a thickness of the electron transport layer 144 may be in a range from 10 Å to about 900 Å, from about 10 Å to about 500 Å or from about 100 Å to about 400 Å.

Within any of the thickness ranges described above, electron injection and electron transport properties may be further improved without an excessive increase in driving voltage, and stability of the electron transfer region 140 may be improved.

Each layer of the electron transfer region 140 may be formed by a process such as a vacuum deposition, a spin coating, an inkjet printing, a laser printing, a casting, a laser thermal transfer, etc.

The light-emitting device ED may further include a capping layer. Light emission efficiency to an outside of the light-emitting device ED may be improved through the capping layer.

As illustrated in FIG. 4, a second capping layer 160b may be formed on an outer surface of the second electrode 150. In some aspects, a first capping layer 160a may be formed on an outer surface of the first electrode 110.

A refractive index of the first capping layer 160a and/or the second capping layer 160b may be 1.6 or more. For example, the refractive index of the first capping layer 160a and/or the second capping layer 160b may be 1.6 or more, 1.8 or more, or 2.0 or more for a light in a wavelength range of 550 nm to 660 nm.

The first capping layer 160a and the second capping layer 160b may each be formed as an organic capping layer including an organic material, an inorganic capping layer including an inorganic material, or an organic-inorganic hybrid capping layer including both the organic and inorganic materials.

The first capping layer 160a and/or the second capping layer 160b may each have a single-layered structure or a multi-layered structure including different materials.

In some aspects, the first capping layer 160a and the second capping layer 160b may each independently include a carbocyclic compound, a heterocyclic compound, an amine group-containing compound, a porphine derivative, a phthalocyanine derivative, a naphthalocyanine derivative, an alkaline metal complex, an alkaline earth metal complex, etc. The first capping layer 160a and the second capping layer 160b may each independently include one of the aforementioned materials, or a combination thereof.

In an aspect, the first capping layer 160a and/or the second capping layer 160b may each independently include an amine group-containing compound.

Referring to FIG. 5, the light-emitting device ED may include a plurality of light-emitting structures (e.g., the light-emitting structures ES1, ES2 and ES3). The light-emitting structures ES1, ES2, and ES3 may each include a stacked structure of the hole transfer region 120, the emission layer 130, and the electron transfer region 140, as described with reference to FIGS. 1 to 4. In some aspects, the light-emitting device ED of FIG. 5 may be a light-emitting device having a tandem structure.

Charge generation layers CGL1 and CGL2 may each be disposed between adjacent structures among the light-emitting structures ES1, ES2 and ES3. Charge generation layers CGL1 and CGL2 may each independently include a p-type charge generation layer and/or an n-type charge generation layer.

The p-type charge generation layer may include a hole transport host compound, such as NPB. For example, the p-type charge generation layer may include a compound represented by Chemical Formula HT as described above. The p-type charge generation layer may further include a p-dopant, such as TCNQ.

The n-type charge generation layer may include an electron transport host compound. For example, the n-type charge generation layer may include a compound represented by Chemical Formula ET as described above. In an aspect, the n-type charge generation layer may include a phenanthroline-based compound.

The charge generation layers CGL1 and CGL2 may include a first charge generation layer CGL1 disposed between the first light-emitting structure ES1 and the second light-emitting structure ES2, and a second charge generation layer CGL2 disposed between the second light-emitting structure ES2 and the third-light emitting structure ES3.

In some aspects, the first light-emitting structure ES1, the first charge generation layer CGL1, the second light-emitting structure ES2, the second charge generation layer CGL2, the third light-emitting structure ES3, and the second electrode 150 may be sequentially stacked on a top surface of the first electrode 110.

Colors emitted from the first light-emitting structure ES1, the second light-emitting structure ES2 and the third light-emitting structure ES3 may be the same or different from each other. In some aspects, the first light-emitting structure ES1, the second light-emitting structure ES2 and the third light-emitting structure ES3 may include a red emission layer, a green emission layer and a blue emission layer, respectively, and a white light-emitting structure may be implemented through the tandem structure, but is not limited thereto.

In FIG. 5, the 3-stack tandem structure in which three light-emitting structures are stacked is illustrated as an example, but the tandem structure of the light-emitting device of the present disclosure is not limited to the structure illustrated in FIG. 5. For example, 2-stack structure, or a 4-stack structure, a 5-stack structure, or more stacked structure as will be described with reference FIG. 6 may also be implemented.

Referring to FIG. 6, as described with reference to FIG. 5, a tandem structure in which the light-emitting structure and a charge generation layer are alternately and repeatedly stacked may be disposed between the first electrode 110 and the second electrode 150.

In some aspects, first to mth light-emitting structures ES1 to ESm may be sequentially stacked from the top surface of the first electrode 110 with the charge generation layer interposed therebetween. The charge generation layer may include a first charge generation layer CGL1 to an (m−1)th charge generation layer CGLm−1 sequentially stacked from the top surface of the first electrode 110.

As illustrated in FIG. 6, the first light-emitting structure ES1, the first charge generation layer CGL1, the second light-emitting structure ES2, the second charge generation layer CGL2, . . . , an (m−1)th light-emitting structure ESm−1, an (m−1)th charge generation layer CGLm−1, an mth light-emitting structure ESm, and the second electrode 150 may be sequentially stacked from the top surface of the first electrode 110.

In some aspects, m is 4, and an intermediate layer of the light-emitting device may have a 4-stack tandem structure, and may include first to fourth light-emitting structures ES1, ES2, ES3 and ES4, and first to third charge generation layers CGL1, CGL2 and CGL3. Colors of light generated from the first to fourth light-emitting structures ES1, ES2, ES3 and ES4 may be the same or different from each other.

In an aspect, the first to fourth light emitting structures ES1, ES2, ES3 and ES4 may include at least one blue light-emitting structure and at least one green-light emitting structure. In a non-limiting example, the first to third light emitting structures ES1, ES2 and ES3 may correspond to the blue light-emitting structure, and the fourth light emitting structure ES4 may correspond to the green-light emitting structure.

In some aspects, m is 5, and an intermediate layer of the light-emitting device may have a 5-stack tandem structure, and may include first to fifth light-emitting structures ES1, ES2, ES3, ES4 and ES5, and first to fourth charge generation layers CGL1, CGL2, CGL3 and CGL4. Colors of light generated from the first to fifth light-emitting structures ES1, ES2, ES3, ES4, and ES5 may be the same or different from each other.

In an aspect, the first to fifth light-emitting structures ES1, ES2, ES3, ES4 and ES5 may include at least one blue light emitting structure and at least one green light emitting structure. In a non-limiting example, the first to fifth light-emitting structures ES1, ES2, ES3, ES4 and ES5 may include three blue light-emitting structures and two green light-emitting structures. For example, the first, third and fifth light-emitting structures ES1, ES3 and ES5 may correspond to the blue light-emitting structure, and the second and fourth light-emitting structures ES2 and ES4 may correspond to the green light-emitting structure.

### Display Device/Electronic Device

The above-described light-emitting device ED may be applied to an electronic device and may be provided as a light-emitting portion or a light-emitting unit of the electronic device.

Examples of an electronic device may include a display device, a billboard, a signboard, a light source, a lighting device, a personal computer such as a laptop computer or a desktop computer, a mobile phone, an electronic book, an electronic dictionary, an electronic notebook, a health-care device including a diagnostic device and various sensors, various display parts for transportation means (automobile, aircraft, ship, train, etc.).

In some aspects, the light-emitting device ED may be applied to an organic light emitting diode (OLED) display device or a quantum dot (QD)—OLED display device.

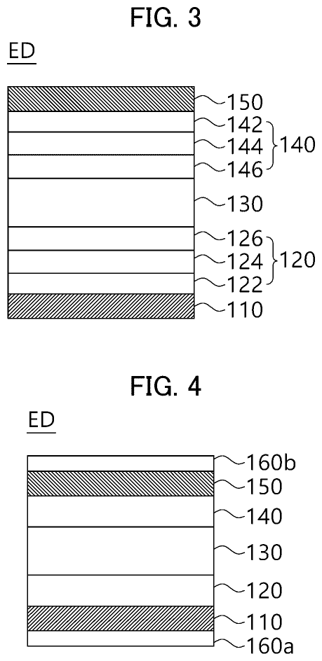

FIG. 7 is a schematic cross-sectional view illustrating a display device in accordance with aspects of the present disclosure.

Referring to FIG. 7, the display device may include a circuit layer CL disposed on a base substrate 200, and light-emitting devices ED1, ED2 and ED3 disposed on the circuit layer CL.

The base substrate 200 may serve as a supporting substrate or as a back-plane substrate of a display device. The base substrate 200 may be a glass substrate or a plastic substrate.

In some aspects, the base substrate 200 may include a polymer material having transparent and flexible properties. When the base substrate 200 includes a polymer material, the base substrate 200 may be used in a transparent flexible display device. For example, the base substrate 200 may include a polymer material such as polyimide, polysiloxane, an epoxy resin, an acrylic resin, polyester, etc. In an aspect, the base substrate 200 may include polyimide.

The circuit layer CL may include transistors TR1, TR2, and TR3. The circuit layer CL may include wiring layers and insulating layers that form a thin film transistor array (TFT-Array).

The circuit layer CL may further include a buffer layer 205 on a top surface of the base substrate 200. The buffer layer 205 may block the penetration of moisture through the base substrate 200, and may also block the diffusion of impurities between the base substrate 200 and the structures formed thereon.

The buffer layer 205 may include, e.g., silicon oxide, silicon nitride, or silicon oxynitride. The buffer layer 205 may include one of the aforementioned materials, or a combination thereof. In some aspects, the buffer layer 205 may have a stacked structure that includes a silicon oxide layer and a silicon nitride layer.

The transistors TR1, TR2, and TR3 may be disposed on the buffer layer 205. A first transistor TR1, a second transistor TR2, and a third transistor TR3 may be electrically connected to a first light-emitting device ED1, a second light-emitting device ED2, and a third light-emitting device ED3, respectively.

The transistors TR1, TR2 and TR3 may each include an active layer 210, a gate insulation layer 220, and a gate electrode 230.

The active layer 210 may be disposed on the buffer layer 205, and may be patterned for each pixel. The active layer 210 may include a silicon compound such as amorphous silicon or polysilicon. A p-type dopant or an n-type dopant may be doped in a region of the active layer 210, and the active layer 210 may include a source region, a drain region, and a channel region.

The active layer 210 may include an oxide semiconductor, such as indium gallium zinc oxide (IGZO), zinc tin oxide (ZTO), or ITZO.

The gate insulation layer 220 may be formed on the active layer 210, and the gate electrode 230 may be stacked on the gate insulation layer 220. As illustrated in FIG. 7, the gate insulation layer 220 may be patterned to partially cover each active layer 210. Alternatively, the gate insulation layer 220 may extend continuously over multiple pixels or light-emitting regions, and may be provided as a common layer for the first, second, and third transistors TR1, TR2 and TR3.

The gate electrode 230 may overlap the channel region of the active layer 210 in a thickness direction.

An insulating interlayer 240 may be formed on the active layer 210 to cover the gate electrode 230 and the gate insulation layer 220. Connection electrodes 250 and 260 which may be in contact with or electrically connected to the active layer 210 may each be disposed on the insulating interlayer 240.

The connection electrodes 250 and 260 may extend through the insulating interlayer 240 to be in contact with or electrically connected to the active layer 210. When the gate insulation layer 220 is provided as a common layer for multiple light-emitting regions, the connection electrodes 250 and 260 may also extend through the gate insulation layer 220.

The connection electrodes 250 and 260 may include a source electrode 250 that may be in contact with or connected to the source region of the active layer 210, and a drain electrode 260 that may be in contact with or connected to the drain region of the active layer 210.

The gate insulation layer 220 and the insulating interlayer 240 may each independently include silicon oxide, silicon nitride, or silicon oxynitride, and may each have a stacked structure that includes a silicon oxide layer and a silicon nitride layer.

The gate electrode 230 and the connection electrodes 250 and 260 may include a metal such as Ag, Mg, Al, W, Cu, Ni, Cr, Mo, Ti, Pt, Ta, Nd, Sc, an alloy thereof, or a nitride thereof.

A via insulation layer 270 may be formed on the insulating interlayer 240 to cover the connection electrodes 250 and 260.

The via insulation layer 270 may accommodate a via structure electrically connecting the first electrode 110 and the drain electrode 260. The via insulation layer 270 may serve as a planarization layer of the circuit layer CL. In aspects, the via insulation layer 270 may include an organic material such as polyimide, an epoxy resin, an acrylic resin, polyester, etc.

The light-emitting devices ED1, ED2, and ED3 may be disposed on the via insulation layer 270. For example, as described with reference to FIGS. 1 to 4, the light-emitting devices ED1, ED2, and ED3 may include the first electrode 110, the hole transfer region 120, the emission layer 130, the electron transfer region 140, and the second electrode 150 which are sequentially stacked from the via insulation layer 270.

The first electrode 110 may be electrically connected to the transistors TR1, TR2 and TR3 or the connection electrodes 250 and 260 in the circuit layer CL through the via structure. As illustrated in FIG. 7, the first electrode 110 may be in contact with or connected to the drain electrode 260 to serve as a pixel electrode patterned for each light-emitting region or pixel.

A pixel defining layer 280 may be formed on the via insulation layer 270 to define each light-emitting region or pixel. A blue light-emitting region, a red light-emitting region, and a green light-emitting region may be separated and defined by the pixel defining layer 280, and the light-emitting devices ED1, ED2, and ED3 may respectively correspond to a blue light-emitting device, a red light-emitting device, and a green light-emitting device.

The pixel defining layer 280 may partially cover the first electrode 110 of each light-emitting region.

As illustrated in FIG. 7, the hole transfer region 120 and the electron transfer region 140 may each be provided as a common layer that continuously extends over the pixel defining layer 280 and the first electrodes 110. The emission layer 130 may be formed within each light emitting-region or pixel, and may be separated by the pixel defining layer 280.

In some aspects, the emission layer 130 may also be provided as a common layer that continuously extends over the light emitting-regions or pixels. In some aspects, the hole transfer region 120, the emission layer 130, and the electron transfer region 140 may each be patterned and separately formed for each light-emitting region or pixel.

The second electrode 150 may be provided as a common electrode that continuously extends over the light-emitting regions or the pixels.

An encapsulation layer 290 may be disposed on the pixel defining layer 280 and the light-emitting devices ED1, ED2, and ED3 to protect the light-emitting devices ED1, ED2 and ED3 from moisture and/or oxygen. The encapsulation layer 290 may be a thin film encapsulation (TFE) having a single-layered structure or multi-layered structure.

The encapsulation layer 290 may include an inorganic layer that includes silicon nitride (SiNₓ), silicon oxide (SiOₓ), indium tin oxide, indium zinc oxide, or any combination thereof; an organic layer that includes polyethylene terephthalate, polyethylene naphthalate, polycarbonate, polyimide, polyethylene sulfonate, polyoxymethylene, polyarylate, hexamethyldisiloxane, an acrylic resin (e.g., polymethylmethacrylate, polyacrylic acid, etc.), an epoxy resin (e.g., an aliphatic glycidyl ether (AGE)) or any combination thereof; or a combination of the inorganic layer and the organic layer.

The display device or the electronic device may further include a functional layer 300 disposed on the encapsulation layer 290. The functional layer 300 may include a sensor layer such as a touch sensor layer, an optical layer such as a polarizing layer, a color conversion layer, a color filter layer, a window film, or any combination thereof.

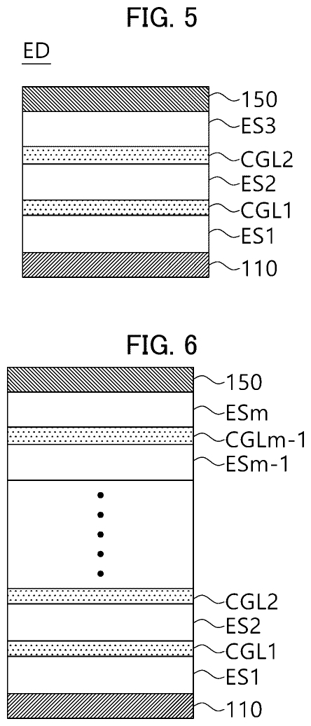

FIG. 8 is a schematic cross-sectional view illustrating a display device in accordance with example aspects.

Referring to FIG. 8, each of the light-emitting devices ED1, ED2 and ED3 may have a tandem structure, e.g., a 2-stack tandem structure.

In some aspects, the hole transfer region 120 and the electron transfer region 140 may be continuously and commonly formed and included in an intermediate layer of each light-emitting structure. Additionally, a charge generation layer CGL may continuously extend across a plurality of pixels and may be commonly included in the intermediate layer of each light-emitting structure.

The first light-emitting device ED1 may include a first lower emission layer 130-1a disposed between the hole transfer region 120 and the charge generation layer CGL, and a first upper emission layer 130-1b disposed between the charge generation layer CGL and the electron transfer region 140.

The second light-emitting device ED2 may include a second lower emission layer 130-2a disposed between the hole transfer region 120 and the charge generation layer CGL, and a second upper emission layer 130-2b disposed between the charge generation layer CGL and the electron transfer region 140.

The third light-emitting device ED3 may include a third lower emission layer 130-3a disposed between the hole transfer region 120 and the charge generation layer CGL, and a third upper emission layer 130-3b disposed between the charge generation layer CGL and the electron transfer region 140.

The lower and upper emission layers included in each light-emitting structure may generate light of the same color. In an aspect, each of the first lower emission layer 130-1a and the first upper emission layer 130-1b included in the first light-emitting device ED1 may correspond to a red emission layer. Each of the second lower emission layer 130-2a and the second upper emission layer 130-2b included in the second light-emitting device ED2 may correspond to a green emission layer. Each of the third lower emission layer 130-3a and the third upper emission layer 130-3b included in the third light-emitting device ED3 may correspond to a blue emission layer.

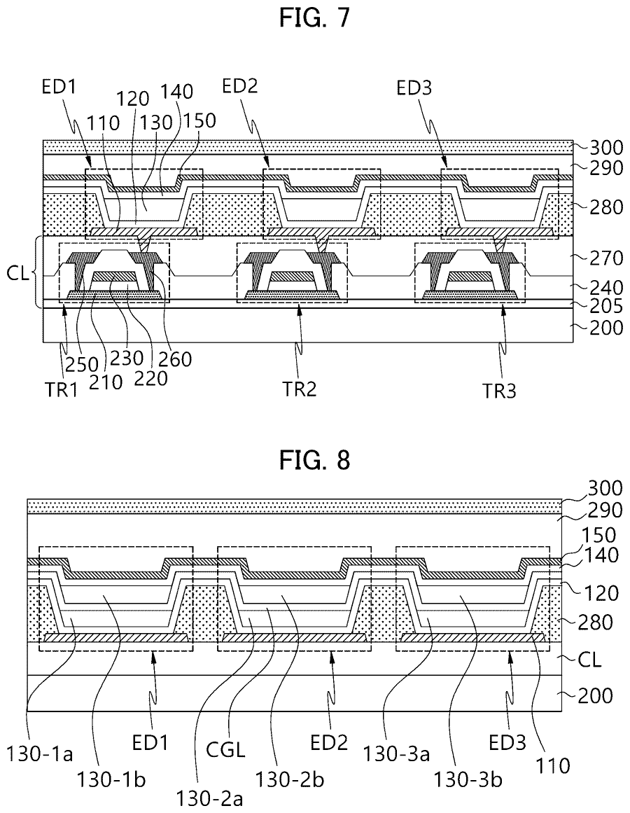

FIG. 9 is a schematic cross-sectional view illustrating a stack construction of light-emitting structure in a display device in accordance with aspects of the present disclosure. For convenience of illustration and description, illustration of the circuit layer, the base substrate, the pixel defining layer, etc., is omitted from FIG. 9, and a shape of each layer or element in the light-emitting structure is briefly shown as a rectangle.

Referring to FIG. 9, at least one of the light-emitting devices ED1, ED2 and ED3 or pixel areas PA1, PA2 and PA3 may have a tandem structure including a plurality of emission layers, and at least one of the remainder may have a single emission layer structure.

In some aspects, one of the light-emitting devices ED1, ED2 and ED3 or the pixel areas PA1, PA2, and PA3 may have a tandem structure, and the remainder may have a single emission layer structure.

As illustrated in FIG. 9, the first light-emitting device ED1, the second light-emitting device ED2, and the third light-emitting device ED3 may be included in the first pixel area PA1, the second pixel area PA2, and the third pixel area PA3, respectively. In some aspects, the first pixel area PA1, the second pixel area PA2, and the third pixel area PA3 may correspond to a red pixel area, a green pixel area, and a blue pixel area, respectively.

The hole transfer region 120, the electron transfer region 140, and the second electrode 150 may each be provided as a common layer continuously extending over the first pixel area PA1, the second pixel area PA2, and the third pixel area PA3.

The first-light emitting device ED1 included in the first pixel area PA1 may include a first emission layer 130-1, and the second light-emitting device ED2 included in the second pixel area PA2 may include a second emission layer 130-2. Each of the first emission layer 130-1 and the second emission layer 130-2 may be a single-layered emission layer.

The third light-emitting device ED3 included in the third pixel area PA3 may have, e.g., a 2-stack tandem structure. The third light-emitting device ED3 may include a third lower emission layer 130-3a and a third upper emission layer 130-3b separated with the charge generation layer CGL interposed therebetween. Each of the third lower emission layer 130-3a and the third upper emission layer 130-3b may correspond to a blue emission layer.

A lower electron transfer region 140a may be disposed between the charge generation layer CGL and the third lower emission layer 130-3a. An upper hole transfer region 120b may be disposed between the charge generation layer CGL and the third upper emission layer 130-3b.

Accordingly, a tandem light-emitting structure in which the first electrode 110, the hole transfer region 120, the third lower emission layer 130-3a, the lower electron transfer region 140a, the charge generation layer CGL, the upper hole transfer region 120b, the third upper emission layer 130-3b, the electron transfer region 140, and the second electrode 150 are sequentially stacked may be disposed in the third pixel area PA3.

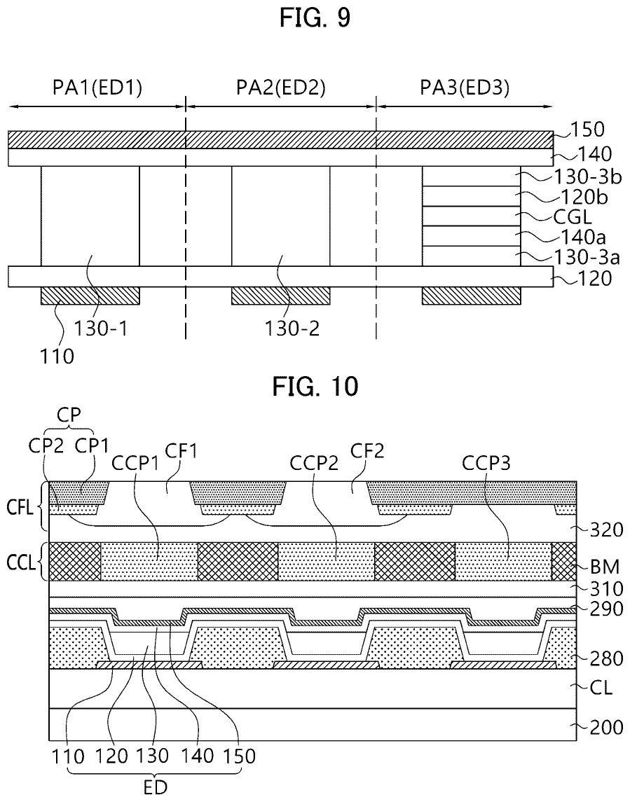

FIG. 10 is a schematic cross-sectional view illustrating a display device in accordance with aspects of the present disclosure.

FIG. 10 illustrates a display device having a QD-OLED structure according to aspects of the present disclosure.

Detailed descriptions regarding elements and structures that are the same as or substantially similar to those described with reference to FIG. 7 will not be repeated here.

Referring to FIG. 10, the pixel defining layer 280 and the light-emitting device ED may be disposed on the circuit layer CL, as described above with reference to FIG. 7. In some aspects, each pixel may emit light of the same wavelength region. In an aspect, each light-emitting device ED may emit a blue light.

In some aspects, each light-emitting region may include the light-emitting device having the tandem structure, as described above with respect to FIG. 5. In this case, the intermediate layer ITL of each light-emitting device ED may be provided as a common layer that continuously extends over a plurality of the light-emitting regions.

A color control layer CCL may be disposed on the encapsulation layer 290, and the color control layer CCL may include color control portions CCP1, CCP2, and CCP3.

The color control portions CCP1, CCP2 and CCP3 may each include a light transformer such as a quantum dot or a phosphor. In each of the color control portions CCP1, CCP2 and CCP3, the light transformer may convert a wavelength of a provided light and emit a resulting light.

The color control portions CCP1, CCP2 and CCP3 may be separated or spaced apart from each other by a bank BM. The bank BM may substantially overlap the pixel defining layer 280, and the color control portions CCP1, CCP2 and CCP3 may substantially overlap each of the emission layers 130.

The color control layer CCL may include a first color control portion CCP1 including a first quantum dot that converts a first color light provided from the light-emitting device ED into a second color light, a second color control portion CCP2 including a second quantum dot that converts the first color light into a third color light, and a third color control portion CCP3 that transmits the first color light.

In some aspects, the first color light, the second color light, and the third color light may be a blue light, a red light, and a green light, respectively. The first quantum dot and the second quantum dot may respectively be a red quantum dot and a green quantum dot.

The color control portions CCP1, CCP2 and CCP3 may each further include a scattering material such as inorganic particles. The third color control portion CCP3 may not include quantum dots and may include the scattering material. The scattering material may include TiO₂, ZnO, Al₂O₃, SiO₂, hollow silica, etc. The scattering material may be one of the aforementioned materials or a combination thereof.

The color control portions CCP1, CCP2, and CCP3 may each further include a binder resin that disperses the quantum dot and the scattering material. The binder resin may include an acrylic resin, a urethane resin, a silicone resin, an epoxy resin, etc.

A color filter layer CFL that includes color filters CF1 and CF2 and a light-shielding portion CP may be disposed on the color control layer CCL.

The color filter layer CFL may include a first filter CF1 that transmits the second color light, a second filter CF2 that transmits the third color light, and a third filter that transmits the first color light. For example, the first filter CF1 may be a red filter, the second filter CF2 may be a green filter, and the third filter may be a blue filter.

The color filters CF1 and CF2 may each include a photosensitive binder resin and a colorant including a pigment and/or a dye. The first filter CF1 may include a red pigment or dye, and the second filter CF2 may include a green pigment or dye.

The light-shielding portion CP may be disposed between the color filters. In some aspects, the light-shielding portion may include a first light-shielding portion CP1 and a second light-shielding portion CP2 that includes colorants of different colors.

In some aspects, the first light-shielding portion CP1 may include a blue colorant, and the second light-shielding portion CP2 may include a red colorant or a black colorant. In an aspect, in the blue light-emitting region, a portion of the first light-shielding portion CP1 may be provided as a blue color filter and may be exposed between the second light-shielding portions CP2, so that an additional color filter (e.g., the third filter) may be omitted.

A first barrier layer 310 may be disposed between the color control layer CCL and the light-emitting device ED (or the encapsulation layer 290). A second barrier layer 320 may be disposed between the color control layer CCL and the color filter layer CFL.

The barrier layers 310 and 320 may each include at least one inorganic layer. For example, the barrier layers 310 and 320 may each independently include silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, etc.

In an aspect, the barrier layers 310 and 320 may each have a multi-layered structure that further includes an organic layer.

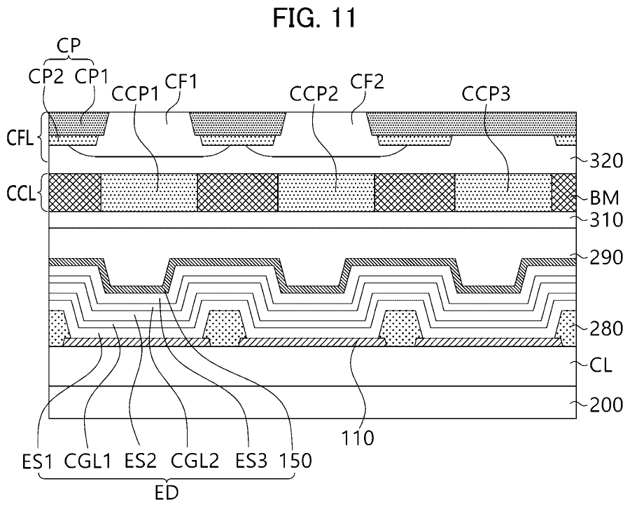

FIG. 11 is a schematic cross-sectional view illustrating a display device in accordance with aspects of the present disclosure. Detailed descriptions of elements and structures substantially the same as or similar to those described with reference to FIG. 10 are omitted herein.

Referring to FIG. 11, the light-emitting device ED corresponding to the color control portions CCP1, CCP2 and CCP3 may be disposed on the first electrode 110 serving as the pixel electrode, and the light-emitting device ED may have a tandem structure.

In some aspects, as described with reference to FIG. 5, the first light-emitting structure ES1, the first charge generation layer CGL1, the second light-emitting structure ES2, the second charge generation layer CGL2, and the third light-emitting structure ES3 may be sequentially stacked between the first electrode 110 and the second electrode 150. The first light-emitting structure ES1, the first charge generation layer CGL1, the second light-emitting structure ES2, the second charge generation layer CGL2, and the third light-emitting structure ES3 may be continuously and commonly formed in a plurality of pixel areas or light-emitting regions.

In an aspect, the first light-emitting structure ES1, the second light-emitting structure ES2, and the third light-emitting structure ES3 may generate different color lights, and the light-emitting device ED may generate a white light. In an aspect, the first light-emitting structure ES1, the second light-emitting structure ES2, and the third light-emitting structure ES3 may all generate blue lights.

In some aspects, as described with reference to FIG. 6, the light-emitting device ED may include a tandem structure of 4-stack, 5-stack, or more of the stacked number.

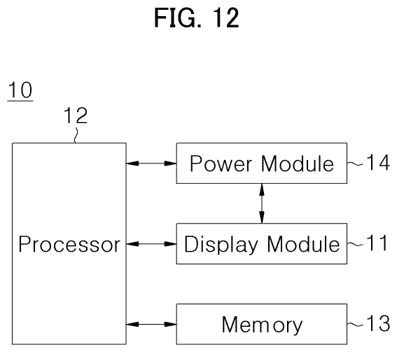

FIG. 12 is a block diagram of an electronic device in accordance with an aspect of the present disclosure.

Referring to FIG. 12, an electronic device 10 according to an aspect may include a display module 11, a processor 12, a memory 13 and a power module 14.

The processor 12 may include a central processing unit (CPU), an application processor (AP), a graphic processing unit (GPU), a communication processor (CP), an image signal processor (ISP) and/or a controller.

Data information for an operation of the processor 12 or the display module 11 may be stored in the memory 13. When the processor 12 executes an application stored in the memory 13, an image data signal and/or an input control signal may be transmitted to the display module 11, and the display module 11 may process the received signal and output image information through a display screen.

The power module 14 may include a power supply module such as a power adapter or a battery device, and a power conversion module that converts a power supplied by the power supply module to a generate power required for the operation of the electronic device 10.

At least one of components of the electronic device 10 as described above may be included in the display device according to the above-described aspects. Additionally, some of individual modules functionally included in one module may be included in the display device, and others may be provided separately from the display device. For example, the display module 11 may include the display device, and the processor 12, the memory 13 and the power module 14 may be provided in the form of another device in the electronic device 10 different from the display device.

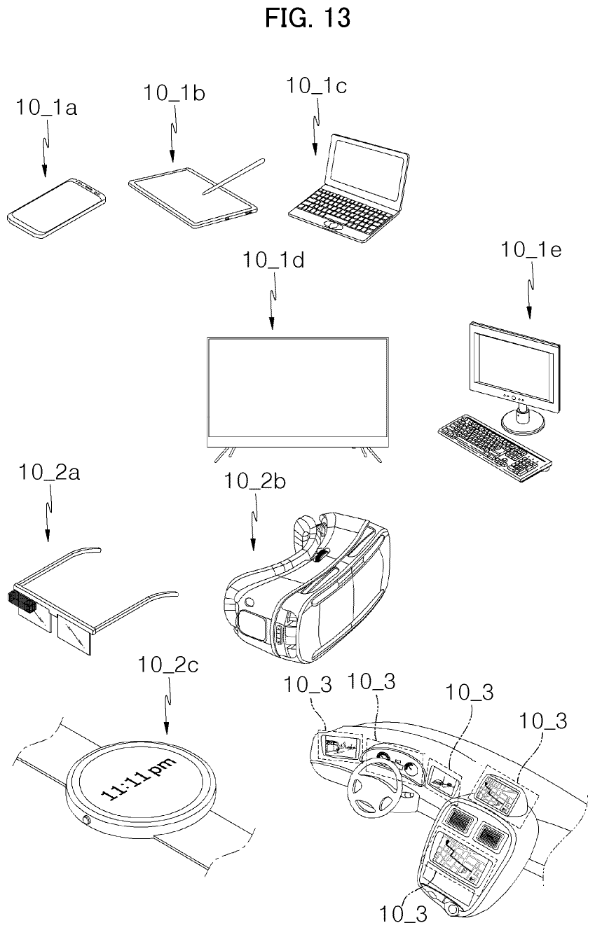

FIG. 13 is a schematic diagram of an electronic device in accordance with various aspects of the present disclosure.

Referring to FIG. 13, non-limiting examples of various electronic devices to which the display device according to the above-described aspects is applied include an electronic device for displaying an image such as a smartphone 10_1a, a tablet PC 10_1b, a laptop 10_1c, a TV 10_1d, a desk monitor 10_1e, or the like; a wearable electronic device including a display module such as smart glasses 10_2a, a head mounted display 10_2b, a smart watch 10_2c, or the like; a vehicle electronic device 10_3 including a display module such as a center information display (CID) disposed at a vehicle instrument panel, a center fascia, a dashboard, etc., a room mirror display, or the like. The electronic device may include a virtual reality glass or an augmented reality glass.

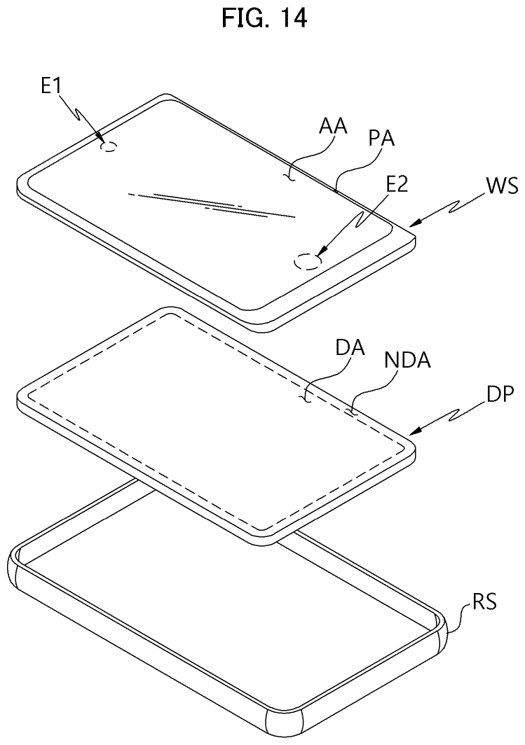

FIG. 14 is a schematic exploded perspective view illustrating an electronic device in accordance with aspects of the present disclosure.

According to some aspects, the electronic device may be implemented in the form of a mobile phone (smart phone), a tablet, a PC, or the like, including the above-described display device.

Referring to FIG. 14, the electronic device may include a window structure WS, a display panel DP, and a rear structure RS.

The window structure WS may provide an external display surface recognized by a user, such as a viewing surface of a mobile phone, and may include a transparent material film. For example, the window structure WS may include glass (e.g., ultra-thin glass (UTG), a hard coating film, a plastic film, or the like.

An outer surface of the window structure WS may include an active area AA and a peripheral area PA. The active area AA may provide a surface from which an image of the display device DD is substantially displayed and to which a user's touch/command is input. The peripheral area PA may substantially correspond to a bezel area of the display device.

The display panel DP may include the above-described display device and may have a display area DA and a non-display area NDA. The display area DA of the display panel DP may substantially correspond to or overlap the active area AA of the window structure WS. The non-display area NDA of the display panel DP may substantially correspond to or overlap the peripheral area PA of the window structure WS.

In some aspects, functional device areas E1 and E2 may be included in the active area AA of the window structure WS. For example, a first functional device area E1 may be included at one end portion of the active area AA and may be implemented, e.g., in the form of a camera hole. The second functional device area E2 may serve as a fingerprint sensing area.

For example, a sensor structure for a touch sensing or a fingerprint sensing may be disposed in the display panel DP or between the window structure WS and the display panel DP.

The rear structure RS may serve as a frame structure or a housing of the display device or the electronic device. A cover panel may be disposed between the rear structure RS and the display panel DP.

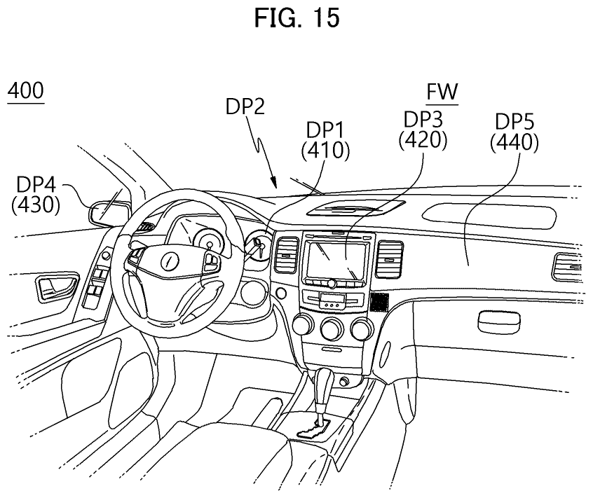

FIG. 15 is a schematic cross-sectional view illustrating an electronic device in accordance with an aspect of the present disclosure.

The electronic device may be installed in, embedded in, attached to, or integrated with a vehicle 400. However, the vehicle 400 is not limited to the aspect illustrated in FIG. 15. Further examples of the vehicle 400 may include a transportation means such as a three-wheeled or four-wheeled vehicle, a construction machine, a two-wheeled vehicle, a motor vehicle, a bicycle, a train, etc. Other examples of the vehicle 400 may include an electric vehicle, a hybrid vehicle, etc.

Referring to FIG. 15, at least one of first to fifth display devices DP1, DP2, DP3, DP4, and DP5 may be applied to the vehicle 400.

In example aspects, the first display device DP1 may be disposed in a cluster area 410. Driving information such as a driving distance and speed, and various warning lights may be displayed in the cluster area 410.

The second display device DP2 may be disposed on a front window FW of the vehicle 400. For example, the second display device DP2 may be installed as a head-up display (HUD).

The third display device DP3 may be disposed on a center fascia 420 of the vehicle 400. In the center fascia 420, a button or a switch for controlling an image display or a music player, an air conditioner, a heater, etc., may be displayed, and vehicle information may be displayed thereon.

The fourth display device DP4 may be applied to side mirrors 430 of the vehicle 400. A side mirror 430 may be installed at each of both sides of an exterior of the vehicle 400, and the fourth display device DP4 may be applied to at least one of the side mirrors 430 installed at each of the both sides.

The fifth display device DP5 may be disposed on a passenger seat dashboard 440. Information/image identical to or different from information/image displayed on the cluster area 410 and/or the center fascia 420 may be displayed at the passenger seat dashboard 440.

### Electronic Apparatus

The above-described light-emitting device ED may be applied to an electronic apparatus, and may serve as a light-emitting portion or a light-emitting unit of the electronic apparatus.

The electronic apparatus may include the light-emitting device ED including the polycyclic compound of the above-described Chemical Formula 1, thereby achieving improved color properties, light emission efficiency and life-span properties.

In some aspects, the electronic apparatus may include the above-described electronic device.

The electronic apparatus may include, e.g., a video wall, a flat panel display, a curved display, a computer monitor, a medical monitor, a television, a billboard, a light for indoor or outdoor lighting and/or signals, a head-up display, a full or partial transparent display, a flexible display, a rollable display, a foldable display, a laser printer, a phone, a mobile phone, a tablet, a phablet, a personal information terminal (PDA), a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.

Hereinafter, an organometallic compound according to an aspect of the present disclosure will be described in detail with reference to the Examples and the Comparative Examples. The Examples are provided to assist in understanding the disclosure, but they are provided as non-limiting examples, and the scope of the disclosure is not limited thereto. It will be clear to those skilled in the art that various changes and modifications to disclosed examples can be made within the scope of the disclosure.

### Synthesis Example

### (1) Synthesis of Compound HT-01

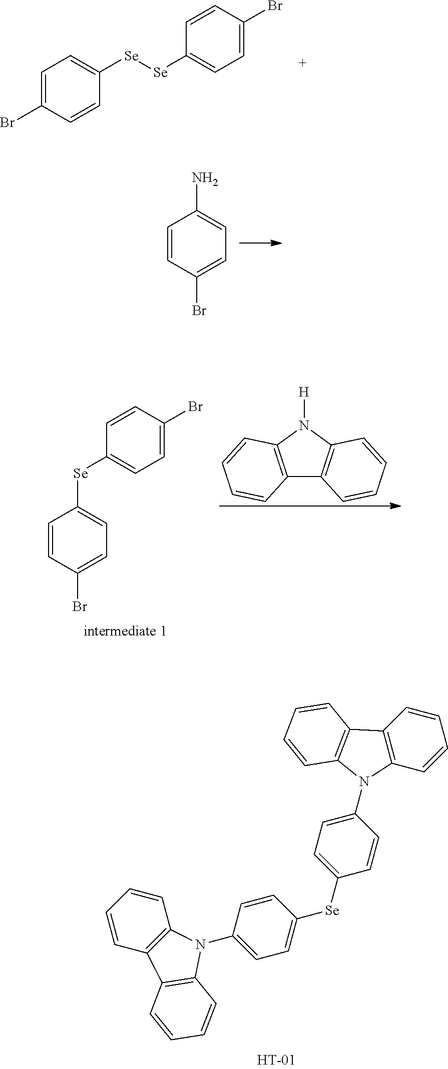 [SMILES: Brc1ccc([Se][Se]c2ccc(Br)cc2)cc1 ; Nc1ccc(Br)cc1 ; Brc1ccc([Se]c2ccc(Br)cc2)cc1 ; c1ccc2c(c1)[nH]c1ccccc12 ; c1ccc2c(c1)c1ccccc1n2-c1ccc([Se]c2ccc(-n3c4ccccc4c4ccccc43)cc2)cc1]

### Synthesis of Intermediate 1

MeCN (0.1M 1 eq reagent standard) was added to a flask containing 1,2-bis(4-bromophenyl)diselane (1 eq) and 4-bromoaniline (2.2 eq), tBuONO (3 eq) was slowly added at 0° C., and then stirred at room temperature for 5 hours. Thereafter, distilled water was added, and extraction with ethyl acetate and washing with distilled water were performed. After drying with MgSO₄ and distillation under reduced pressure, a residue was separated by a column to synthesize an intermediate 1 (yield 62.3%). The intermediate 1 was confirmed by LC-MS. (C₁₂H₈Br₂Se:[M]+390.05)

### 2) Synthesis of Compound HT-01

In a flask containing the intermediate 1 (1 eq), carbazole (2.2 eq), and Cs₂CO₃ (2.5 eq), DMF (0.1M 1 eq reagent standard) was added, and then refluxed and stirred for 18 hours. The solution was cooled to room temperature, extracted with MC (methylene chloride), and washed with distilled water. After drying with MgSO₄ and distillation under reduced pressure, a residue was separated with a column to obtain a compound HT-01 (yield 75.9%). The compound HT-01 was confirmed by LC-MS and ¹H-NMR as described in Table 1 below.

### (2) Synthesis of Compound HT-07

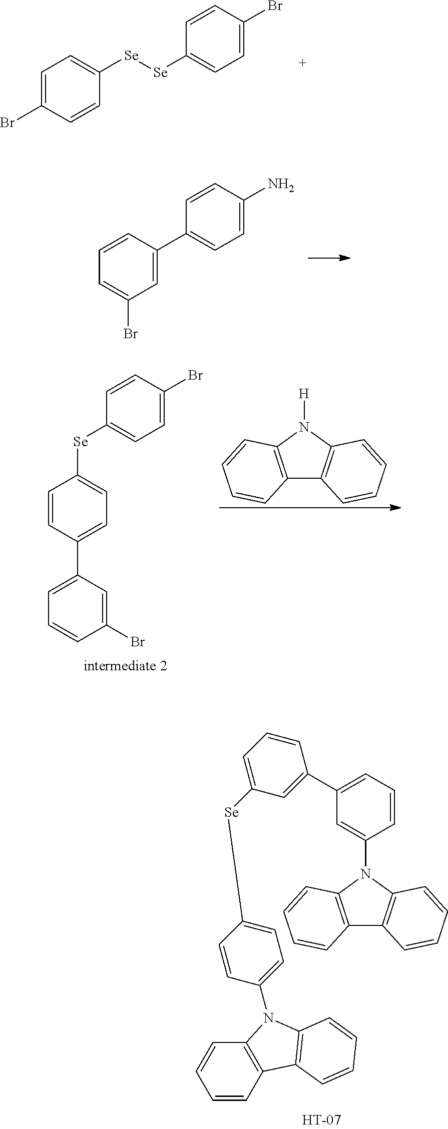

### 1) Synthesis of Intermediate 2

MeCN (0.1M 1 eq reagent standard) was added to a flask containing 1,2-bis(4-bromophenyl)diselane (1 eq) and 3′-bromo-[1,1′-biphenyl]-4-amine (2.2 eq), tBuONO (3 eq) was slowly added at 0° C., and the stirred at room temperature for 5 hours. Distilled water was added, and extraction with ethyl acetate and washing with distilled water were performed. After drying with MgSO₄ and distillation under reduced pressure, a residue was separated by a column to synthesize an intermediate 2 (yield 69.1%). The intermediate 2 was confirmed by LC-MS. (C₁H₁₂Br₂Se: [M]+467.15)

### 2) Synthesis of HT-07

DMF (0.1M 1 eq reagent standard) was added to a flask containing the intermediate 2 (1 eq), carbazole (2.2 eq), and Cs₂CO₃ (2.5 eq), and the mixture was refluxed for 18 hours. The mixture was cooled to room temperature, extracted with MC (methylene chloride), and washed with distilled water. After drying with MgSO₄ and distillation under reduced pressure, a residue was separated through a column to obtain a compound HT-07 (yield 73.2%). The compound HT-07 was confirmed by LC-MS and 1H-NMR as described in Table 1 below.

TABLE 1

| | | MS/FAB | |
|---|---|---|---|
| No. | ¹H-NMR (CDCl₃) | found | calc. |
| HT-01 | 7.16 (t, 2H), 7.21 (t, | 564.1 | 563.57 |
| | 2H), 7.35-7.60 (m, | | |
| | 10H), 7.65-7.95 (m, | | |
| | 6H), 8.15 (d, 2H), | | |
| | 8.6 (d, 2H) | | |
| HT-07 | 7. 16 (m, 2H), 7.21 (m, | 640.1 | 639.67 |
| | 2H), 7.35-7.60 (m, | | |
| | 12H), 7.65 (s, 1H), | | |
| | 7.69 (t, 1H), 7.75- | | |
| | 8.0 (m, 6H), 8.15- | | |
| | 8.6 (m, 4H) | | |

### Fabrication of Light-Emitting Device

An ITO glass substrate was cut into a size of 50 mm×50 mm×0.5 mm, and the cut substrate was ultrasonically cleaned for 10 minutes using isopropyl alcohol and pure water. The ultrasonically cleaned substrate was irradiated with an ultraviolet ray for 10 minutes and exposed to ozone, and then mounted on a vacuum deposition device.

m-MTDATA was vacuum-deposited as a hole injection layer of a thickness of 40 Å on a top surface of the glass substrate, and then NPB was vacuum-deposited to a thickness of 10 Å to form a hole transport layer. Host-1 and Host-2 materials were mixed in a weight ratio of 5:5, and Dopant 1 material was simultaneously deposited to form an emission layer with a thickness of 300 Å on the hole transport layer. ETL1 was deposited on the emission layer to form an electron transport layer of a thickness of 300 Å, and then Al was vacuum-deposited to a thickness of 1200 Å to form a cathode, thereby manufacturing a light-emitting device. Structures of the compounds used in the manufacture of the light-emitting device are as follows.

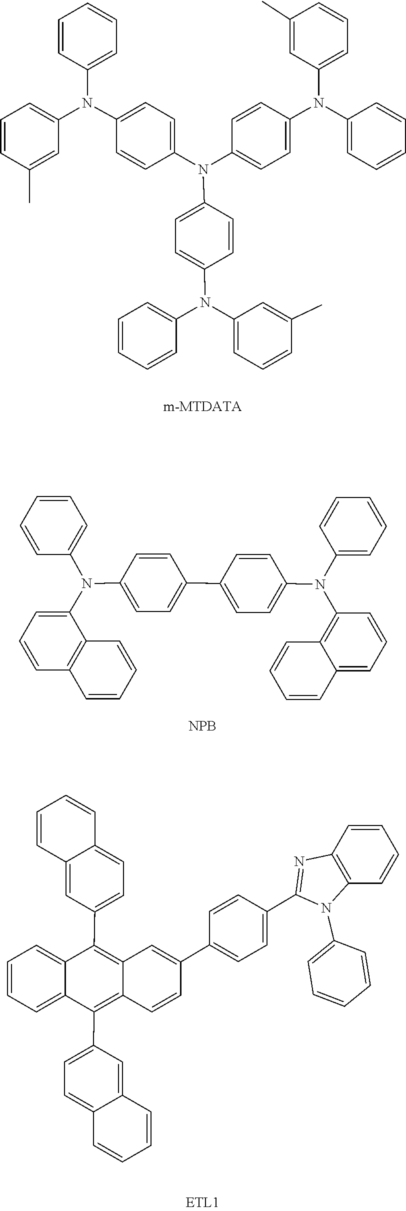 [SMILES: Cc1cccc(N(c2ccccc2)c2ccc(N(c3ccc(N(c4ccccc4)c4cccc(C)c4)cc3)c3ccc(N(c4ccccc4)c4cccc(C)c4)cc3)cc2)c1 ; c1ccc(N(c2ccc(-c3ccc(N(c4ccccc4)c4cccc5ccccc45)cc3)cc2)c2cccc3ccccc23)cc1 ; "NPB" ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

### Evaluation Example

Properties of the light-emitting device were measured using the following methods, and the results are shown in Table 2.

1) A driving voltage and an efficiency (cd/A) at a current density of 10 mA/cm² were measured using the V7000 OLED IVL Test System, (Polaronix).

2) The light-emitting device was continuously driven at a current density of 10 mA/cm², and a time until a luminance dropped from 95% of an initial value was measured. A relative value with respect to the time measured in the light-emitting device using the compound of Comparative Example 1 was expressed as a life-span of each light-emitting device.

TABLE 2

| | | | | | life- |
|---|---|---|---|---|---|
| | | | | efficiency | span |
| | Dopant | Host-1 | Host-2 | (cd/A) | (T₉₅) |
| Example 1 | Dopant- | HT-01 | ET-01 | 26.1 | 72.3 |
| | 1 | | | | |
| Example 2 | Dopant- | HT-01 | ET-04 | 26.9 | 70.1 |
| | 1 | | | | |
| Example 3 | Dopant- | HT-01 | ET-07 | 27.1 | 73.1 |
| | 1 | | | | |
| Example 4 | Dopant- | HT-07 | ET-01 | 28.1 | 72.9 |
| | 1 | | | | |
| Example 5 | Dopant- | HT-07 | ET-04 | 27.6 | 67.1 |
| | 1 | | | | |
| Example 6 | Dopant- | HT-07 | ET-07 | 28.2 | 67.8 |
| | 1 | | | | |
| Example 7 | Dopant- | HT-01 | ET-01 | 28.3 | 69.1 |
| | 2 | | | | |
| Example 8 | Dopant- | HT-01 | ET-04 | 28.1 | 68.9 |
| | 2 | | | | |
| Example 9 | Dopant- | HT-01 | ET-07 | 27.9 | 68.7 |
| | 2 | | | | |
| Example 10 | Dopant- | HT-07 | ET-01 | 27.6 | 68.6 |
| | 2 | | | | |
| Example 11 | Dopant- | HT-07 | ET-04 | 27.9 | 69.2 |
| | 2 | | | | |
| Example 12 | Dopant- | HT-07 | ET-07 | 28.1 | 70.3 |
| | 2 | | | | |
| Comparative | Dopant- | HT-A | ET-07 | 24.1 | 35.1 |
| Example 1 | 1 | | | | |
| Comparative | Dopant- | HT-A | — | 21.3 | 25.5 |
| Example 2 | 1 | | | | |
| Comparative | Dopant- | HT-B | ET-07 | 23.9 | 36.1 |
| Example 3 | 2 | | | | |
| Comparative | Dopant- | HT-B | — | 20.9 | 30.7 |
| Example 4 | 2 | | | | |

The compounds shown in Table 2 are as follows.

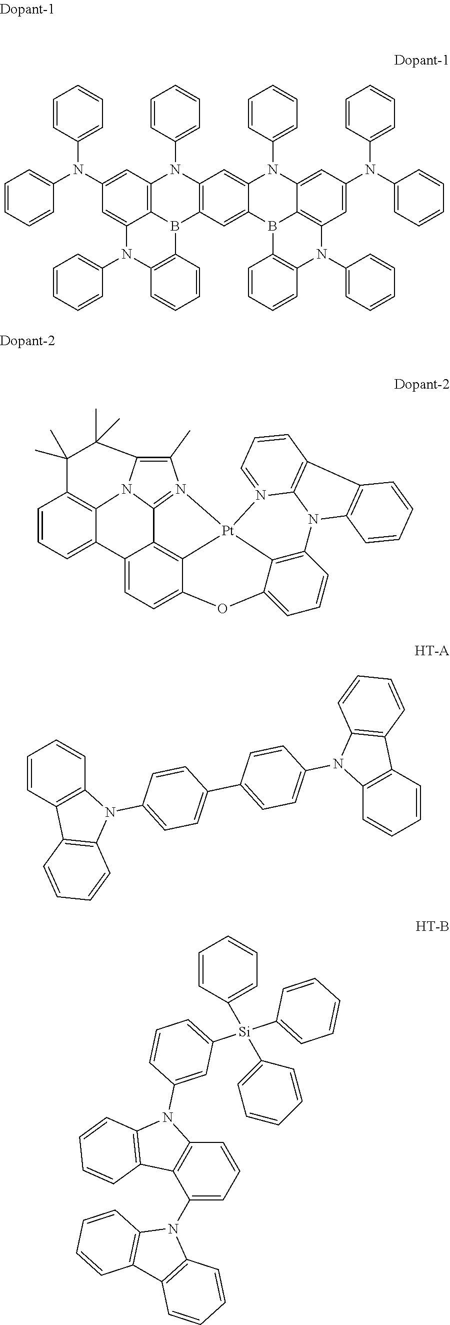

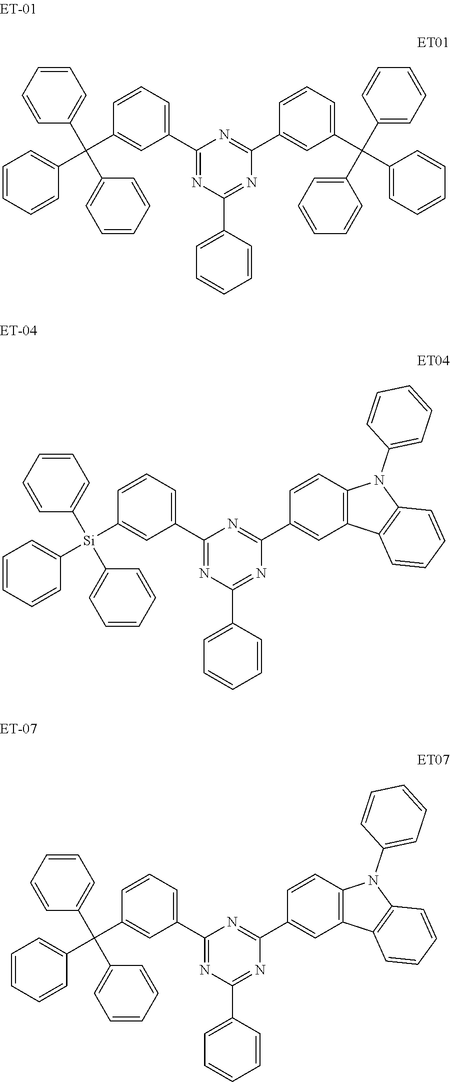 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3ccc4c(c3)c3ccccc3n4-c3ccccc3)n2)cc1 ; c1ccc(-c2nc(-c3cccc(C(c4ccccc4)(c4ccccc4)c4ccccc4)c3)nc(-c3ccc4c(c3)c3ccccc3n4-c3ccccc3)n2)cc1]

Referring to Table 2, in Examples where the composition for forming an emission layer according to aspects of the present disclosure was applied provided improved luminescence efficiency and life-span compared to those from Comparative Examples.

As described above, the composition for forming an emission layer according to aspects of the present disclosure may be used to control electrons in the emission layer. Accordingly, formation of excitons in the emission layer may be accelerated, so that luminescence efficiency may be improved and life-span properties may also be by a current reduction at the same luminance.

What is claimed is:

1. A composition for forming an emission layer, the composition comprising a compound represented by Chemical Formula 1 and a compound represent by Chemical Formula 2: 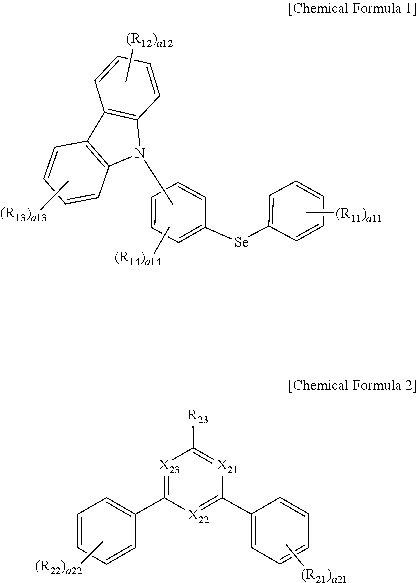
wherein, in Chemical Formula 1 and Chemical Formula 2, X₂₁ to X₂₃ are each independently C(R_{1b}) or N,
R_{1b} is hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₈-C₆₀ condensed polycyclic group, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₁ to R₁₄, and R₂₁ to R₂₃ are each independently hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, —Se(Q₁), —Si(Q₁)(Q₂)(Q₃), —N(Q₁)(Q₂), —B(Q₁)(Q₂), —C(═O)(Q₁), —S(═O)₂(Q₁) or —P(═O)(Q₁)(Q₂), or two or more adjacent ones of R₁₁ to R₁₄, and R₂₁ to R₂₃ are combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₃-C₆₀ heterocycloalkenyl ring, a substituted or unsubstituted C₃-C₆₀ aryl ring or a substituted or unsubstituted C₂-C₆₀ heteroaryl ring,
Q₁ to Q₃ are each independently hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, or a substituted or unsubstituted C₈-C₆₀ a condensed polycyclic group,
a11, a21, and a22 are each independently an integer from 0 to 5, and a12 to a14 are each independently an integer from 0 to 4, and
when one or more of a11 to a14, a21, or a22 are 2 or more, multiple R₁₁ to R₁₄, R₂₁, or R₂₂ are each independently the same or different from each other.

2. The composition for forming the emission layer of claim 1, wherein R₁₁ to R₁₄ and R₂₁ to R₂₃ are each independently hydrogen, deuterium, a substituted or unsubstituted C₁-C₆₀ alkyl group, a substituted or unsubstituted C₂-C₆₀ heteroaryl group, —Se(Q₁) or —Si(Q₁)(Q₂)(Q₃), or two or more adjacent ones of R₁₁ to R₁₄, and R₂₁ to R₂₃ are combined with each other to form a substituted or unsubstituted C₂-C₆₀ heteroaryl ring.

3. The composition for forming the emission layer of claim 1, wherein Q₁ to Q₃ are each independently a substituted or unsubstituted C₆-C₆₀ aryl group or a substituted or unsubstituted C₂-C₆₀ heteroaryl group.

4. The composition for forming the emission layer of claim 1, wherein X₂₁ to X₂₃ are N.

5. The composition for forming the emission layer of claim 1, wherein R₂₃ is any one of Chemical Formula 2A-1, Chemical Formula 2A-2, Chemical Formula 2A-3, Chemical Formula 2A-4, Chemical Formula 2A-5, or Chemical Formula 2A-6: 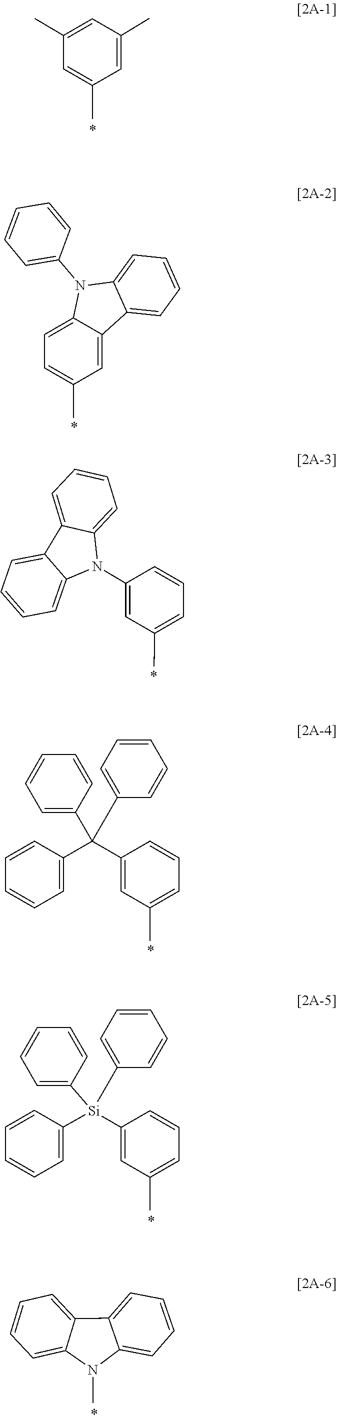
wherein, in Chemical Formula 2A-1, Chemical Formula 2A-2, Chemical Formula 2A-3, Chemical Formula 2A-4, Chemical Formula 2A-5, and Chemical Formula 2A-6, * indicates a bonding position.

6. The composition for forming the emission layer of claim 1, wherein Chemical Formula 1 is any one of Chemical Formula 1-1, Chemical Formula 1-2, Chemical Formula 1-3, Chemical Formula 1-4, Chemical Formula 1-5, Chemical Formula 1-6, Chemical Formula 1-7, or Chemical Formula 1-8: 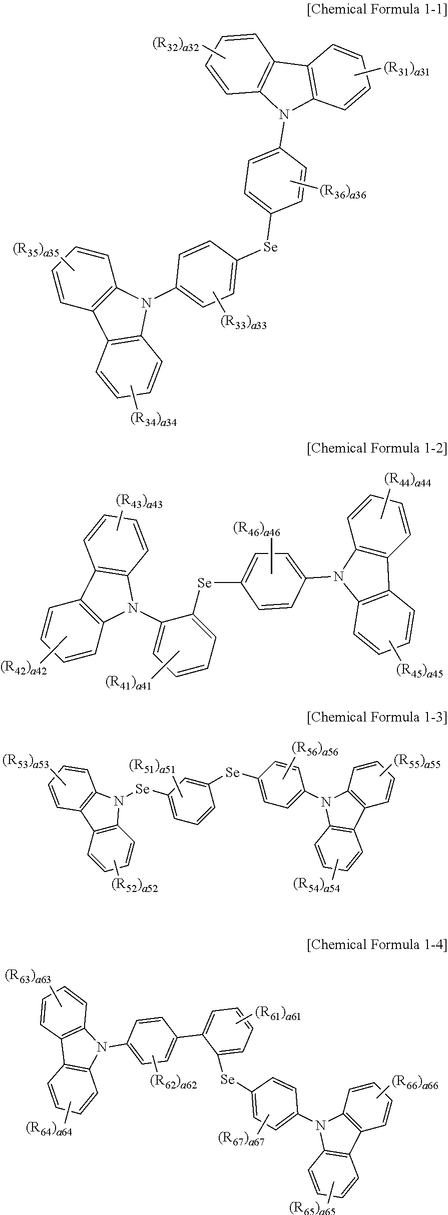 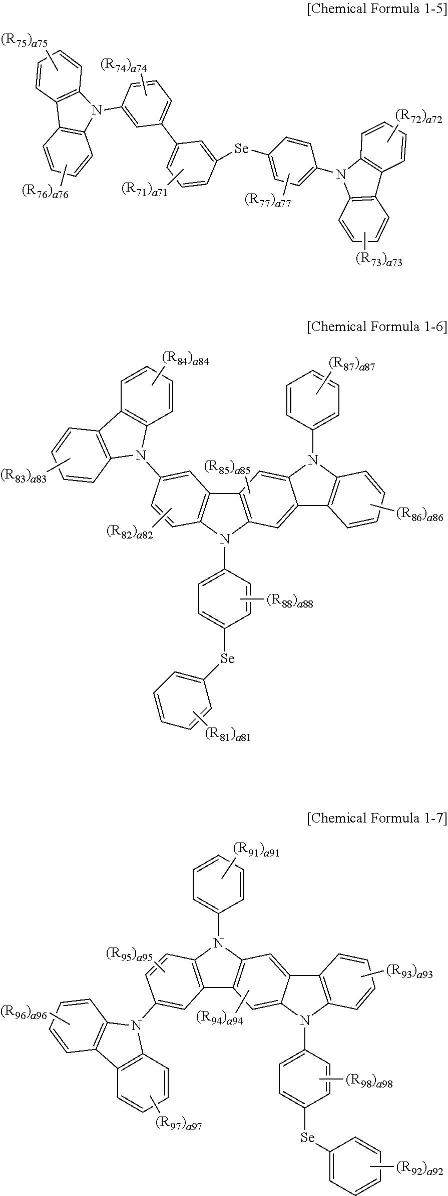 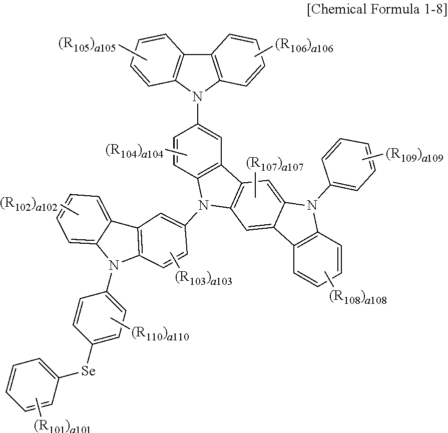
wherein, in Chemical Formula 1-1, Chemical Formula 1-2, Chemical Formula 1-3, Chemical Formula 1-4, Chemical Formula 1-5, Chemical Formula 1-6, Chemical Formula 1-7, and Chemical Formula 1-8, R₃₁ to R₃₆, R₄₁ to R₄₆, R₅₁ to R₅₆, R₆₁ to R₆₇, R₇₁ to R₇₇, R₈₁ to R₈₈, R₉₁ to R₉₈, and R₁₀₁ to R₁₁₀ are each independently hydrogen, deuterium, a substituted or unsubstituted C₁-C₆₀ alkyl group, a substituted or unsubstituted C₂-C₆₀ heteroaryl group, —Se(Q₁) or —Si(Q₁)(Q₂)(Q₃), or two or more of R₃₁ to R₃₆, R₄₁ to R₄₆, R₅₁ to R₅₆, R₆₁ to R₆₇, R₇₁ to R₇₇, R₈₁ to R₈₈, R₈₁ to R₈₈, and R₁₀₁ to R₁₁₀ are combined with each other to form a substituted or unsubstituted C₂-C₆₀ heteroaryl ring,
a31 to a36, a41 to a46, a51 to a56, a61 to a67, a71 to a77, a81, a83, a84, a86, a93, a96 to a98, a102, a105, a106, a108 and a110 are each independently an integer from 0 to 4,
a82, a95, a103, and a104 are each independently an integer from 0 to 3,
a85, a94, and a107 are each independently an integer from 0 to 2, and
a87, a91, a92, a101 and a109 are each independently an integer from 0 to 5,
wherein, when one or more of a31 to a36, a41 to a46, a51 to a56, a61 to a67, a71 to a77, a81 to a88, a91 to a98, or a101 to a110 is 2 or more, multiple R₃₁ to R₃₆, R₄₁ to R₄₆, R₅₁ to R₅₆, R₆₁ to R₆₇, R₇₁ to R₇₇, R₈₁ to R₈₈, R₉₁ to R₉₈, or R₁₀₁ to R₁₁₀ are each independently the same or different from each other.

7. The composition for forming the emission layer of claim 1, wherein R₁₁ to R₁₄ are each independently hydrogen, deuterium, a substituted or unsubstituted phenyl group, a substituted or unsubstituted carbazole group, a substituted or unsubstituted indolecarbazole group or —Se(Q₁), or two or more adjacent ones among R₁₁ to R₁₄ are combined with each other to form a substituted or unsubstituted carbazole group.

8. The composition for forming the emission layer of claim 1, wherein R₂₁ to R₂₃ are each independently hydrogen, deuterium, a substituted or unsubstituted methyl group, —Si(Q₁)(Q₂)(Q₃) or a substituted or unsubstituted carbazole group, or two or more adjacent ones among R₂₁ to R₂₃ are combined with each other to form a substituted or unsubstituted carbazole group.

9. The composition for forming the emission layer of claim 1, wherein Chemical Formula 2 is any one of Chemical Formula 2-1, Chemical Formula 2-2, or Chemical Formula 2-3: 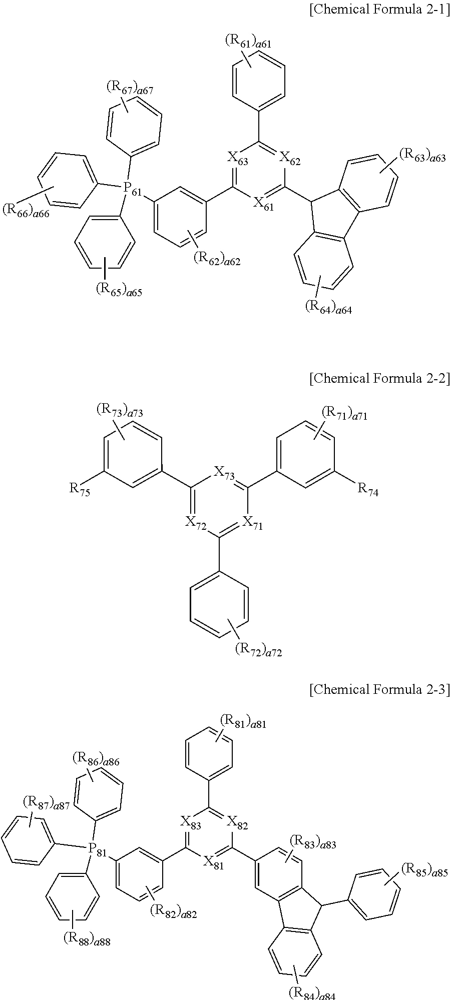
wherein, in Chemical Formula 2-1, Chemical Formula 2-2, and Chemical Formula 2-3, X₆₁ to X₆₃, X₇₁ to X₇₃ and X₈₁ to X₈₃ are each independently C(R_{1b}) or N,
R_{1b} is hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or an unsubstituted C₆-C₆₀ aryloxy group, a substituted or unsubstituted C₈-C₆₀ condensed polycyclic group, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
P₆₁ and P₈₁ are each independently C, Si or Ge,
R₆₁ to R₆₇, R₇₁ to R₇₅ and R₈₁ to R₈₈ are each independently hydrogen, deuterium, a substituted or unsubstituted C₁-C₆₀ alkyl group, a substituted or unsubstituted C₂-C₆₀ heteroaryl group or —Si(Q₁)(Q₂)(Q₃), or two or more adjacent ones of R₆₁ to R₆₇, R₇₁ to R₇₅, and R₈₁ to R₈₈ are combined with each other to form a substituted or unsubstituted C₂-C₆₀ heteroaryl ring,
Q₁ to Q₃ are each independently hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, or a substituted or unsubstituted C₈-C₆₀ condensed polycyclic ring,
a61, a65 to a67, a72, a81, and a85 to a88 are each independently an integer from 0 to 5,
a62 to a64, a71, a73, a82 and a84 are each independently an integer from 0 to 4, and
a83 is an integer from 0 to 3,
wherein, when one or more of a61 to a67, a71 to a73, or a81 to a88 are 2 or more, multiple R₆₁ to R₆₇, R₇₁ to R₇₅, or R₈₁ to R₈₈ are each independently the same or different from each other.

10. The composition for forming the emission layer of claim 9, wherein at least one of R₇₁, R₇₃, R₇₄ and R₇₅ is any one of Chemical Formula 2-2A-1, Chemical Formula 2-2A-2, Chemical Formula 2-2A-3, or Chemical Formula 2-2A-4: 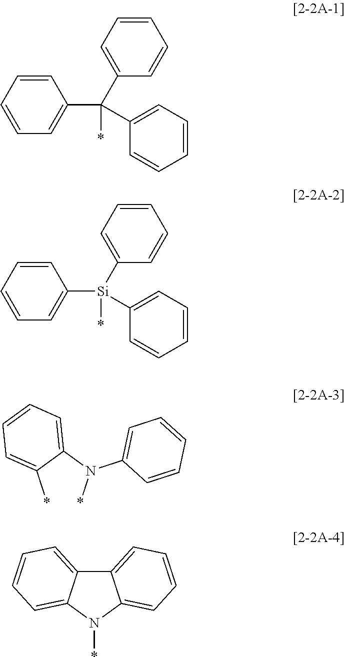
wherein, in Chemical Formula 2-2A-1, Chemical Formula 2-2A-2, Chemical Formula 2-2A-3, and Chemical Formula 2-2A-4, * is a bonding position.

11. The composition for forming the emission layer of claim 1, wherein the compound represented by Chemical Formula 1 is a compound represented by any one of Chemical Formula 1-9, Chemical Formula 1-10, Chemical Formula 1-11, Chemical Formula 1-12, Chemical Formula 1-13, Chemical Formula 1-14, Chemical Formula 1-15, Chemical Formula 1-16, or Chemical Formula 1-17:

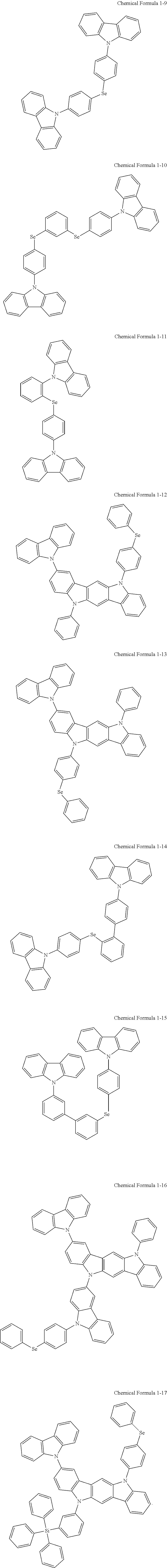

12. The composition for forming the emission layer of claim 1, wherein the compound represented by Chemical Formula 2 is a compound represented by any one of Chemical Formula 2-4, Chemical Formula 2-5, Chemical Formula 2-6, Chemical Formula 2-7, Chemical Formula 2-8, Chemical Formula 2-9, Chemical Formula 2-10, Chemical Formula 2-11, Chemical Formula 2-12, Chemical Formula 2-13, Chemical Formula 2-14, or Chemical Formula 2-15:

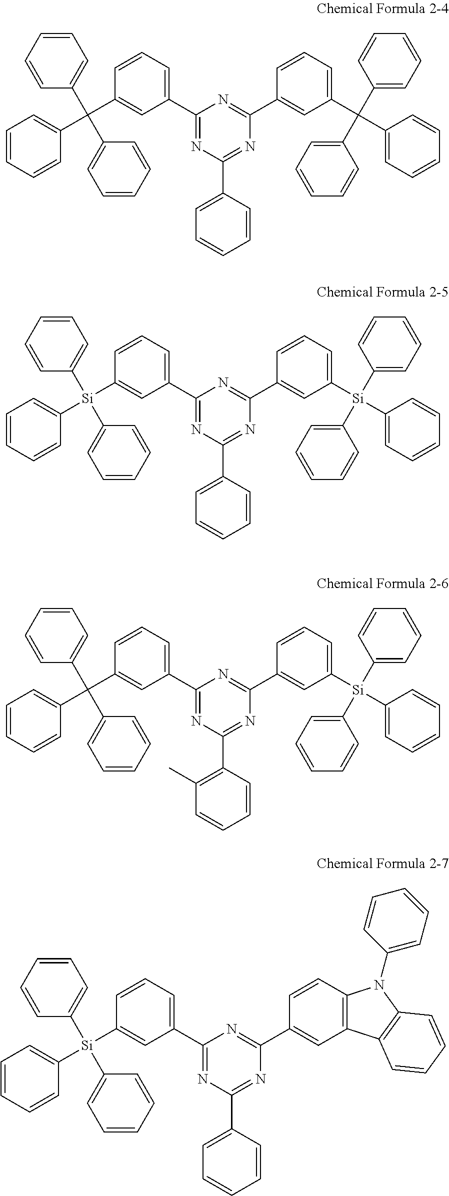 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc([Si](c3ccccc3)(c3ccccc3)c3ccccc3)c2)n1 ; c1ccc(-c2nc(-c3cccc([Si](c4ccccc4)(c4ccccc4)c4ccccc4)c3)nc(-c3ccc4c(c3)c3ccccc3n4-c3ccccc3)n2)cc1]

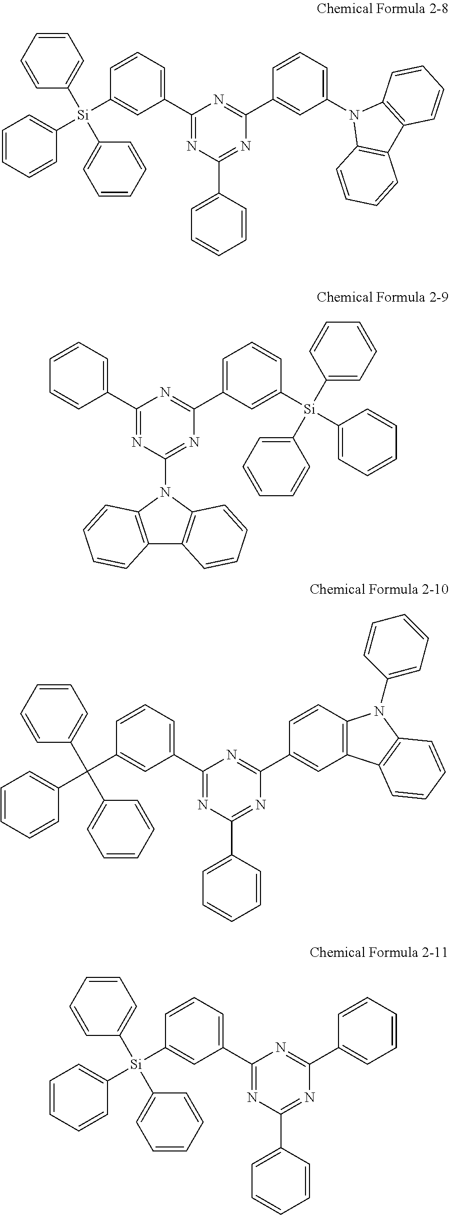 [SMILES: c1ccc(-c2nc(-c3cccc(-n4c5ccccc5c5ccccc54)c3)nc(-c3cccc([Si](c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-n3c4ccccc4c4ccccc43)n2)cc1 ; c1ccc(-c2nc(-c3cccc(C(c4ccccc4)(c4ccccc4)c4ccccc4)c3)nc(-c3ccc4c(c3)c3ccccc3n4-c3ccccc3)n2)cc1 ; c1ccc(-c2nc(-c3ccccc3)nc(-c3cccc([Si](c4ccccc4)(c4ccccc4)c4ccccc4)c3)n2)cc1]

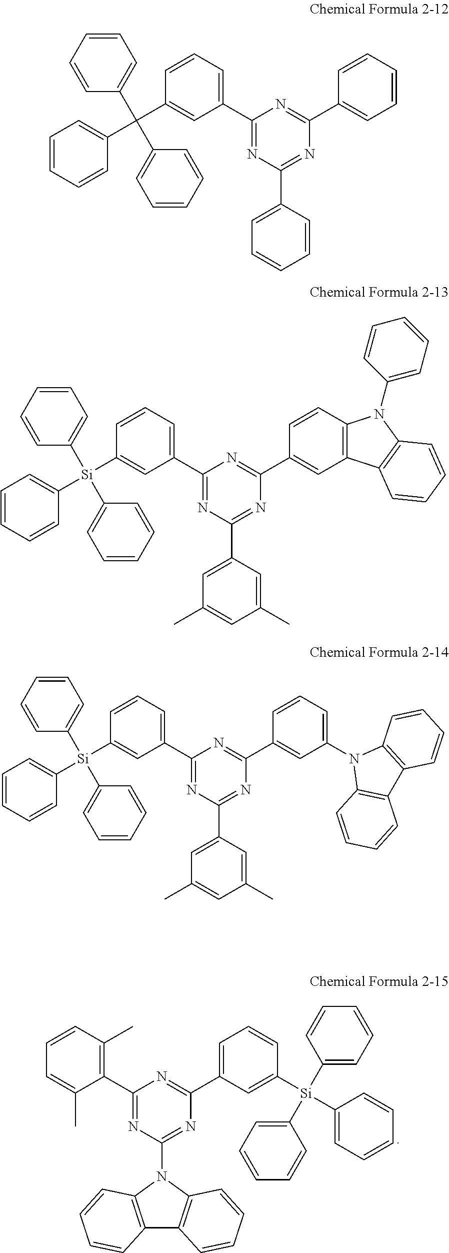 [SMILES: c1ccc(-c2nc(-c3ccccc3)nc(-c3cccc(C(c4ccccc4)(c4ccccc4)c4ccccc4)c3)n2)cc1 ; Cc1cc(C)cc(-c2nc(-c3cccc([Si](c4ccccc4)(c4ccccc4)c4ccccc4)c3)nc(-c3ccc4c(c3)c3ccccc3n4-c3ccccc3)n2)c1 ; Cc1cc(C)cc(-c2nc(-c3cccc(-n4c5ccccc5c5ccccc54)c3)nc(-c3cccc([Si](c4ccccc4)(c4ccccc4)c4ccccc4)c3)n2)c1 ; Cc1cccc(C)c1-c1nc(-c2cccc([Si](c3ccccc3)(c3ccccc3)c3ccccc3)c2)nc(-n2c3ccccc3c3ccccc32)n1 ; "."]

13. A light-emitting device comprising:
a first electrode;
a second electrode; and
an emission layer disposed between the first electrode and the second electrode, the emission layer comprising a composition for forming the emission layer which comprises a compound represented by Chemical Formula 1 and a compound represent by Chemical Formula 2: 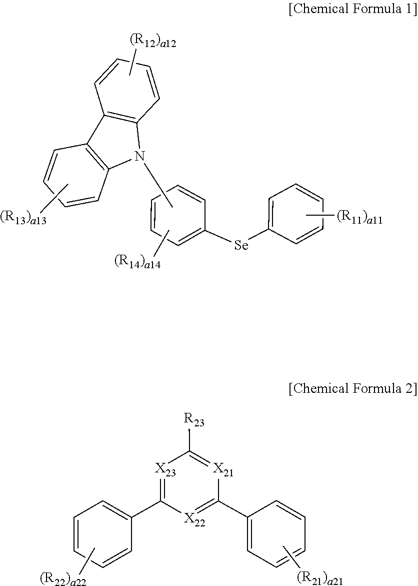
wherein, In Chemical Formula 1 and Chemical Formula 2, X₂₁ to X₂₃ are each independently C(R_{1b}) or N,
R_{1b} is hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₈-C₆₀ condensed polycyclic group, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₁₁ to R₁₄, and R₂₁ to R₂₃ are each independently hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, —Se(Q₁), —Si(Q₁)(Q₂)(Q₃), —N(Q₁)(Q₂), —B(Q₁)(Q₂), —C(═O)(Q₁), —S(═O)₂(Q₁) or —P(═O)(Q₁)(Q₂), or two or more adjacent ones of R₁₁ to R₁₄, and R₂₁ to R₂₃ are combined with each other to form a substituted or unsubstituted C₃-C₆₀ cycloalkyl ring, a substituted or unsubstituted C₅-C₆₀ cycloalkenyl ring, a substituted or unsubstituted C₃-C₆₀ heterocycloalkyl ring, a substituted or unsubstituted C₃-C₆₀ heterocycloalkenyl ring, a substituted or unsubstituted C₃-C₆₀ aryl ring or a substituted or unsubstituted C₂-C₆₀ heteroaryl ring,
Q₁ to Q₃ are each independently hydrogen, deuterium, a halogen, a hydroxyl group, a cyano group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₆₀ cycloalkyl group, a substituted or unsubstituted C₅-C₆₀ cycloalkenyl group, a substituted or unsubstituted C₃-C₆₀ heterocycloalkyl group, a substituted or unsubstituted C₃-C₆₀ heterocycloalkenyl group, a substituted or unsubstituted C₆-C₆₀ aryl group, a substituted or unsubstituted C₂-C₆₀ heteroaryl group, a substituted or unsubstituted C₆-C₆₀ aryloxy group, a substituted or unsubstituted C₆-C₆₀ arylthio group, or a substituted or unsubstituted C₈-C₆₀ a condensed polycyclic group,
a11, a21, and a22 are each independently an integer from 0 to 5, and a12 to a14 are each independently an integer from 0 to 4, and
when one or more of a11 to a14, a21, or a22 are 2 or more, multiple R₁₁ to R₁₄, R₂₁, or R₂₂ are each independently the same or different from each other.

14. The light-emitting device of claim 13, wherein the composition for forming the emission layer is a host for a blue phosphorescent device or a thermally activated delayed fluorescence (TADF) host.

15. The light-emitting device of claim 14, wherein the emission layer further comprises a dopant.

16. The light-emitting device of claim 13, wherein the compound represented by Chemical Formula 1 is a compound represented by any one of Chemical Formula 1-9, Chemical Formula 1-10, Chemical Formula 1-11, Chemical Formula 1-12, Chemical Formula 1-13, Chemical Formula 1-14, Chemical Formula 1-15, Chemical Formula 1-16, or Chemical Formula 1-17: 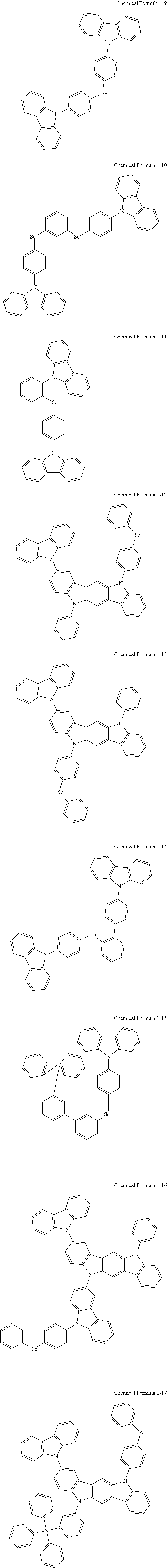
and wherein the compound represented by Chemical Formula 2 is a compound represented by any one of Chemical Formula 2-4, Chemical Formula 2-5, Chemical Formula 2-6, Chemical Formula 2-7, Chemical Formula 2-8, Chemical Formula 2-9, Chemical Formula 2-10, Chemical Formula 2-11, Chemical Formula 2-12, Chemical Formula 2-13, Chemical Formula 2-14, or Chemical Formula 2-15:

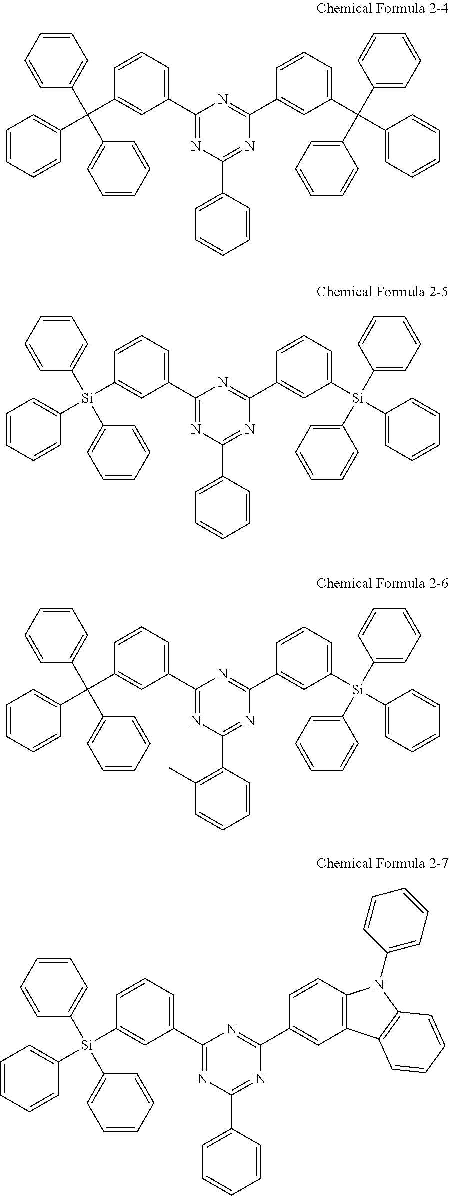 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc([Si](c3ccccc3)(c3ccccc3)c3ccccc3)c2)n1 ; c1ccc(-c2nc(-c3cccc([Si](c4ccccc4)(c4ccccc4)c4ccccc4)c3)nc(-c3ccc4c(c3)c3ccccc3n4-c3ccccc3)n2)cc1]

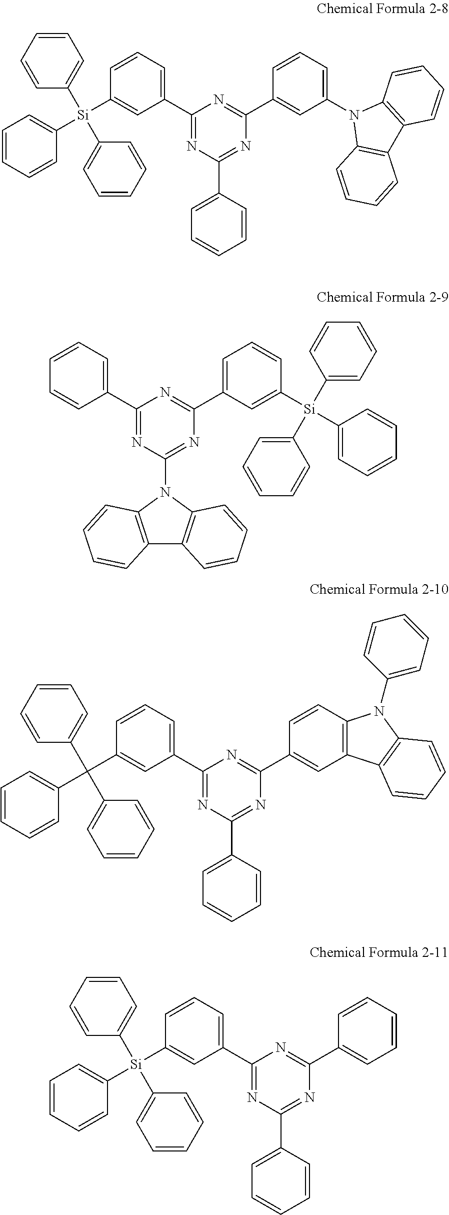 [SMILES: c1ccc(-c2nc(-c3cccc(-n4c5ccccc5c5ccccc54)c3)nc(-c3cccc([Si](c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-n3c4ccccc4c4ccccc43)n2)cc1 ; c1ccc(-c2nc(-c3cccc(C(c4ccccc4)(c4ccccc4)c4ccccc4)c3)nc(-c3ccc4c(c3)c3ccccc3n4-c3ccccc3)n2)cc1 ; c1ccc(-c2nc(-c3ccccc3)nc(-c3cccc([Si](c4ccccc4)(c4ccccc4)c4ccccc4)c3)n2)cc1]

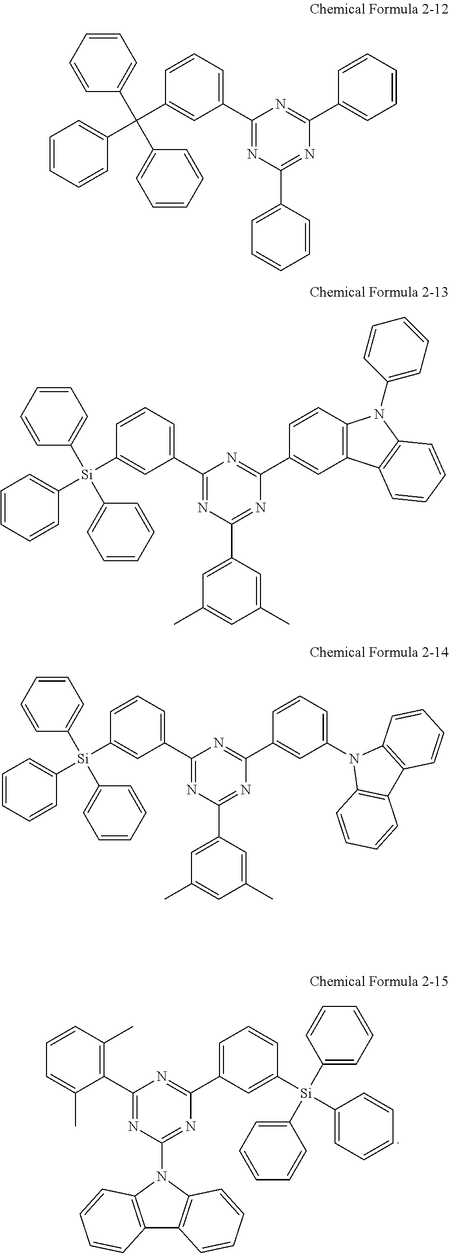 [SMILES: c1ccc(-c2nc(-c3ccccc3)nc(-c3cccc(C(c4ccccc4)(c4ccccc4)c4ccccc4)c3)n2)cc1 ; Cc1cc(C)cc(-c2nc(-c3cccc([Si](c4ccccc4)(c4ccccc4)c4ccccc4)c3)nc(-c3ccc4c(c3)c3ccccc3n4-c3ccccc3)n2)c1 ; Cc1cc(C)cc(-c2nc(-c3cccc(-n4c5ccccc5c5ccccc54)c3)nc(-c3cccc([Si](c4ccccc4)(c4ccccc4)c4ccccc4)c3)n2)c1 ; Cc1cccc(C)c1-c1nc(-c2cccc([Si](c3ccccc3)(c3ccccc3)c3ccccc3)c2)nc(-n2c3ccccc3c3ccccc32)n1 ; "."]

17. The light-emitting device of claim 13, wherein a weight ratio of the compound represented by Chemical Formula 1 and the compound represented by Chemical Formula 2 in the composition for forming the emission layer is from 1:9 to 9:1.

18. The light-emitting device of claim 13, wherein the emission layer comprises a plurality of emission layers, and at least one of the plurality of emission layers comprises the composition for forming the emission layer.

19. An electronic device comprising the light-emitting device of claim 13.

20. The electronic device of claim 19, wherein the electronic device is one of a flat panel display, a curved display, a computer monitor, a medical monitor, a television, a billboard, a light for indoor or outdoor lighting and/or signals, a head-up display, a full or partial transparent display, a flexible display, a rollable display, a foldable display, a stretchable display, a laser printer, a phone, a mobile phone, a tablet, a phablet, a personal information terminal (PDA), a wearable device, a laptop computer, a digital camera, a camcorder, a viewfinder, a micro display, a 3D display, a virtual reality or augmented reality display, a vehicle, a video wall including multiple displays tiled together, a theater or stadium screen, a phototherapy device, or a signage.
